(19) United States
(12) Patent Application Publication
(10) Pub. No.: US 2021/0358571 A1
(43) Pub. Date: Nov. 18, 2021

(54) SYSTEMS AND METHODS FOR PREDICTING PATHOGENIC STATUS OF FUSION CANDIDATES DETECTED IN NEXT GENERATION SEQUENCING DATA

(71) Applicant: Tempus Labs, Inc., Chicago, IL (US)

(72) Inventors: Sumaiya Islam, Chicago, IL (US); Robert Huether, Chicago, IL (US)

(21) Appl. No.: 17/302,867

(22) Filed: May 13, 2021

Related U.S. Application Data
(60) Provisional application No. 63/024,312, filed on May 13, 2020.

(51) Int. Cl. G16B 40/00; G16H 70/60; G06K 9/62; G06N 20/00

(52) U.S. Cl. CPC G16B 40/00; G16H 70/60; G06N 20/00; G06K 9/6259; G06K 9/6288

(57) ABSTRACT

A method of categorizing fusions is provided by the present disclosure. The method includes receiving labeled fusion data including at least one of DNA data or RNA data including at least one detected fusion associated with a specimen, providing the labeled fusion data to a classifier trained to generate a pathogenicity metric corresponding to pathogenicity of each detected fusion, receiving at least one pathogenicity metric from the classifier, and generating a report including one or more detected fusions included in the at least one detected fusion based on the pathogenicity metrics.

### CROSS-REFERENCE TO RELATED APPLICATIONS

This application claims the benefit of U.S. Application No. 63/024,312, filed May 13, 2020, which is incorporated in its entirety herein by reference.

### BACKGROUND

At present, fusion prioritization for reporting is a labor-intensive step, requiring manual curation from existing public databases and/or internally-generated lists.

There is a need for automated prioritization of potential driver fusions in a clinical laboratory workflow. Additionally, there is a need for new and improved discovery methods to identify novel driver fusions in cancer and other conditions.

### SUMMARY OF DISCLOSURE

Disclosed herein are systems, methods, and mechanisms useful for ranking and/or determining pathogenicity for fusions.

In some embodiments, a method of categorizing fusions is provided by the present disclosure. The method includes receiving labeled fusion data including at least one of DNA data or RNA data including at least one detected fusion associated with a specimen, providing the labeled fusion data to a classifier trained to generate a pathogenicity metric corresponding to pathogenicity of each detected fusion, receiving at least one pathogenicity metric from the classifier, and generating a report including one or more detected fusions included in the at least one detected fusion based on the pathogenicity metrics.

In some embodiments, a fusion categorization system including at least one processor and at least one memory is provided by the present disclosure. The system is configured to receive labeled fusion data including at least one of DNA data or RNA data including at least one detected fusion associated with a specimen, provide the labeled fusion data to a classifier trained to generate a pathogenicity metric corresponding to pathogenicity of each detected fusion, receive at least one pathogenicity metric from the classifier, and generate a report including one or more detected fusions included in the at least one detected fusion based on the pathogenicity metrics.

In some embodiments, a method of categorizing fusions is provided by the present disclosure. The method includes receiving labeled fusion data including at least one of DNA data or RNA data including at least one detected fusion associated with a patient, providing the labeled fusion data to a classifier trained to generate a pathogenicity metric corresponding to pathogenicity of each detected fusion, receiving at least one pathogenicity metric from the classifier, and generating a report including one or more detected fusions included in the at least one detected fusion based on the pathogenicity metrics.

### BRIEF DESCRIPTION OF DRAWINGS

The file of this patent contains at least one drawing/photograph executed in color. Copies of this patent with color drawing(s)/photograph(s) will be provided by the Office upon request and payment of the necessary fee.

FIG. 1A shows a block diagram illustrating a system in accordance with some implementations.

FIG. 1B shows a system that includes a patient data store.

FIG. 2A shows an example molecular and clinical data analysis workflow to generate a clinical report.

FIG. 2B shows a distributed diagnostic environment.

FIG. 3 shows an example workflow for nucleic acid sequencing.

FIG. 4A shows an example bioinformatics pipeline.

FIG. 4B shows an exemplary isolate analysis pipeline.

FIG. 5 shows an exemplary process to rank fusion events.

FIG. 6 shows an example clinical report.

FIG. 7 shows an exemplary process to train a classifier.

FIG. 8A shows an exemplary pipeline for fusion scoring and categorization.

FIG. 8B shows exemplary RNA fusion data for a single case processed by a bioinformatics pipeline.

FIG. 8C shows exemplary logic for annotation of genomic start/end and breakpoints for 5′ and 3′ gene partners based on a gene's strandedness.

FIG. 8D shows an example of a list of labeled fusion data.

FIG. 8E shows an exemplary output file resulting from a fusion classifier pipeline.

FIG. 8F shows another exemplary output file resulting from a fusion classifier pipeline.

FIG. 9 shows a classifier in FIG. 8A.

FIG. 10 shows a plot visualizing the categorization of fusions described in FIG. 8A.

FIG. 11A shows an example of positive control testing sets used to calculate performance metrics for a trained classifier.

FIG. 11B shows an example of negative control testing sets used to calculate performance metrics for a trained classifier.

FIG. 11C shows possible strand status of partner sequences.

FIG. 12A shows another example of positive control testing sets used to calculate performance metrics for a trained classifier.

FIG. 12B shows another example of negative control testing sets used to calculate performance metrics for a trained classifier.

FIG. 12C shows an example of performance metric values plotted for tested DriverScore thresholds.

FIG. 13A shows an example of comparing trained classifier output to thresholds to categorize fusions.

FIG. 13B shows a confusion matrix used for calculating performance metrics for a trained classifier.

FIG. 14A shows an example of comparing from analyzing a group of fusion candidates with a trained classifier.

FIG. 14B shows another example of comparing trained classifier output to thresholds to categorize fusions.

FIG. 15 shows a summary of output from a trained classifier for approximately four hundred and six specimens associated with a combined total of approximately thirty-two hundred fusion events.

### DETAILED DESCRIPTION

Definitions

“Artifact” means a false detection of a sequencing variant, which may be due to a sequencing error, bioinformatic software detection error, or another error.

“Breakpoint” means a boundary between two non-contiguous regions in a reference genome that appear to be contiguous in one or more sequencing reads.

“Canonical” means an observable occurrence (for example, a fusion or other genetic variant) that has been documented, published, or otherwise well-established by expert sources or consensus guidelines.

“Driver” or “pathogenic” means having an effect on or contributing to disease pathology.

“Fusion” or “gene fusion” means two genetic sequences that are contiguous in nucleic acid molecules of a specimen but would not be contiguous in a reference genome or in nucleic acid molecules collected from many of the individuals in the population. A gene fusion involves the aberrant juxtaposition of two genetic sequences that can generate a single hybrid RNA transcript and/or chimeric protein product. A fusion can result from structural rearrangements like translocations, inversions, insertions or deletions, transcriptional read-through of neighboring gene sequences, or the splicing of RNA molecules.

“Passenger” or “benign” means having little or no contribution to disease pathology. This can describe fusions occurring in healthy or normal tissue, which may arise due to transcriptional read-through events by the RNA polymerase enzyme, reverse transcriptase template switching artifacts and errors in the downstream analysis of the sequencing reads.

“Whole transcriptome” refers to the coding and non-coding RNA and/or gene expression heterogeneity in cells, tissues, organs and/or an entire body.

“Whole transcriptome sequencing” or “whole transcriptome profile” refers to an effort to capture a whole transcriptome.

Overview

Given observation of a gene fusion, systems and methods are disclosed herein to predict whether a fusion is biologically relevant (for example, if the fusion is a driver fusion versus passenger event or artifact) and/or clinically-actionable. Using feature annotation based on fusion sequence analysis, a model may be employed to score such feature information in order to prioritize fusions for reporting and/or follow-up.

Gene fusions can serve as key drivers in the development of various cancers and represent important therapeutic targets and diagnostic biomarkers. Due to high detection of candidate fusions from RNA-sequencing data and DNA-sequencing data, there is a recognized need to build tools that will make reasonable and automated predictions to identify clinically or biologically relevant fusion events in a tumor sample. The systems and methods disclosed herein include a computational pipeline which scores and prioritizes all detected fusions within a sample to determine which fusions are likely driver events in the tumor. In one embodiment, the pipeline implements a categorization scheme that bins all scored fusion events into Low, Medium and High Confidence levels based on threshold read support levels and a DriverScore metric, which is derived from a binary classification algorithm using specific features, including reading frame, breakpoint region, kinase domain and transcript isoform. In one embodiment, the systems and methods systematically analyzed 3200 fusion candidates from a previously published cohort of 500 paired tumor-normal samples sequenced with the Tempus xT assay. Through use of the systems and methods, 1.7% and 20.1% of fusion candidates were categorized in the High and Medium Confidence levels, respectively, while 78.2% of fusion events were deprioritized as Low Confidence calls. Of the 35 clinically-relevant fusions, 27 (77.1%) were captured in a prioritized set (High/Medium Confidence), including National Comprehensive Cancer Network (NCCN) actionable gene rearrangements involving RET, STAT6 and FUS, while the remaining 8 were assigned as Low Confidence due to an out of frame fusion transcript and insufficient read support. The frequency of prioritized fusions varied by cancer type, with prostate and breast cancer having the highest frequency of prioritized fusions. In addition to well-established canonical fusions, the systems and methods also characterized novel fusions, identifying a subset of 21 novel prioritized fusions which were also observed in The Cancer Genome Atlas tumor samples. Within this subset, 3% of fusion candidates contained a druggable domain such as a tyrosine kinase or Ras-binding domain, signifying the potential of categorization to enable novel fusion drug target discovery. Overall, our analysis highlights the utility of using an automated prioritization tool to detect known canonical fusion drivers and explore novel fusion drug targets and biomarkers.

The systems and methods disclosed herein may be implemented using a molecular pathology system, such as the one disclosed in U.S. Pre-Grant Publication No. 2021/0090694, published Mar. 25, 2021, the contents of which are incorporated herein by reference in their entirety. Briefly, a tumor specimen may be sequenced using, for instance, next-generation sequencing technologies to identify a DNA and/or RNA profile of the specimen.

System Overview

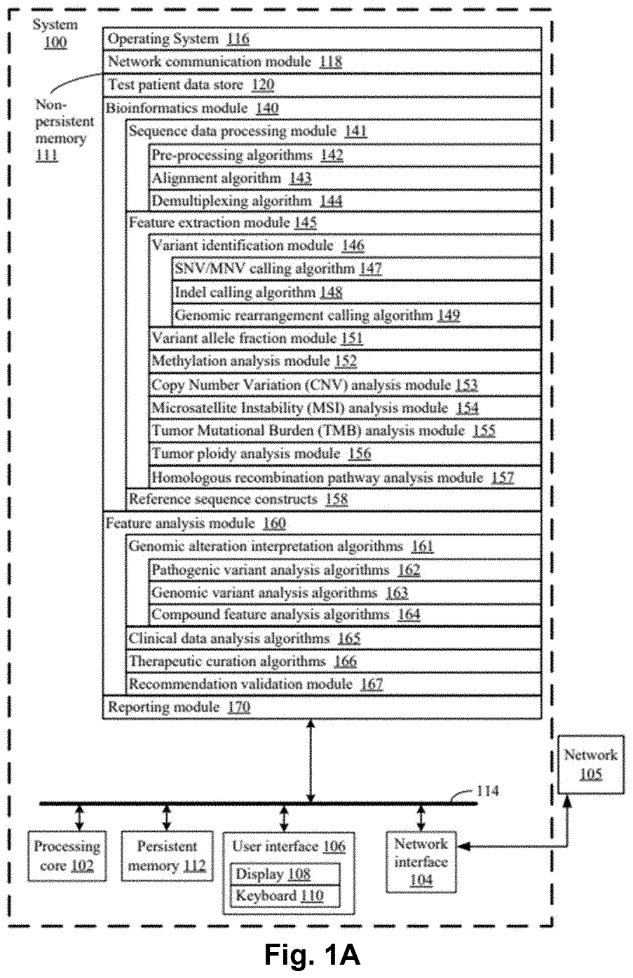

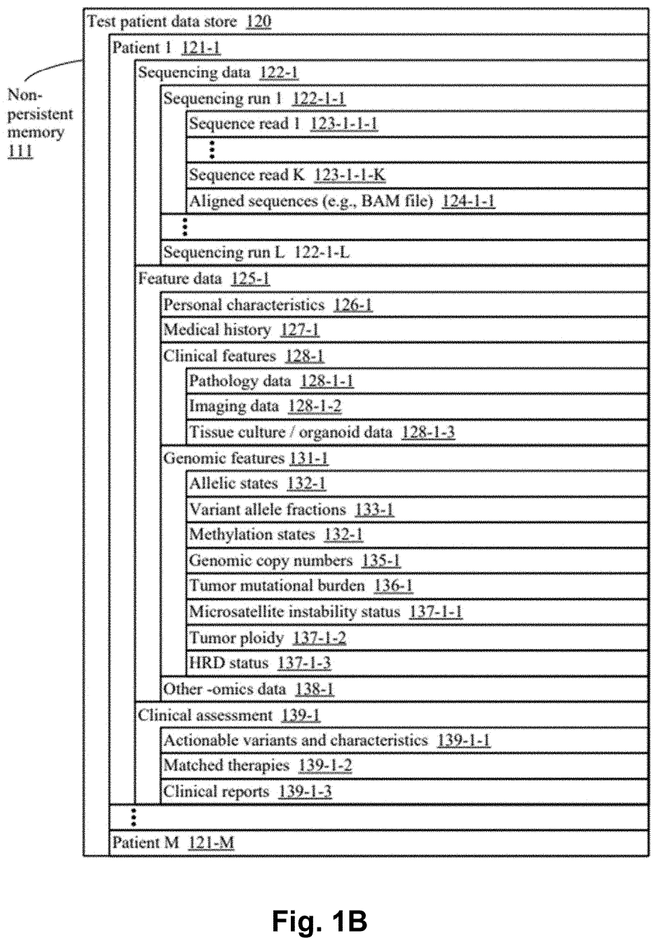

FIG. 1A is a block diagram illustrating a system in accordance with some implementations. The device 100 in some implementations includes one or more processing units CPU(s) 102 (also referred to as processors), one or more network interfaces 104, a user interface 106, for example, including a display 108 and/or an input 110 (for example, a mouse, touchpad, keyboard, etc.), a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112: an operating system 116, which includes procedures for handling various basic system services and for performing hardware dependent tasks; a network communication module (or instructions) 118 for connecting the system 100 with other devices and/or a communication network 105; a test patient data store 120 for storing one or more collections of features from patients (for example, subjects); a bioinformatics module 140 for processing sequencing data and extracting features from sequencing data, for example, from liquid biopsy, solid tumor, or other sequencing assays, including next generation sequencing assays; a feature analysis module 160 for evaluating patient features, for example, genomic alterations, compound genomic features, and clinical features; and a reporting module 180 for generating and transmitting reports that provide clinical support for personalized cancer therapy.

Although FIGS. 1A-1B depict a “system 100,” the figures are intended more as a functional description of the various features that may be present in computer systems than as a structural schematic of the implementations described herein. In practice, and as recognized by those of ordinary skill in the art, items shown separately could be combined and some items could be separated. Moreover, although FIGS. 1A-1B depict certain data and modules in non-persistent memory 111, some or all of these data and modules may be in persistent memory 112. For example, in various implementations, one or more of the above identified elements are stored in one or more of the previously mentioned memory devices, and correspond to a set of instructions for performing a function described above. The above identified modules, data, or programs (for example, sets of instructions) need not be implemented as separate software programs, procedures, datasets, or modules, and thus various subsets of these modules and data may be combined or otherwise re-arranged in various implementations.

In some implementations, the non-persistent memory 111 optionally stores a subset of the modules and data structures identified above. Furthermore, in some embodiments, the memory stores additional modules and data structures not described above. In some embodiments, one or more of the above-identified elements is stored in a computer system, other than that of system 100, that is addressable by system 100 so that system 100 may retrieve all or a portion of such data when needed.

For purposes of illustration in FIG. 1A, system 100 is represented as a single computer that includes all of the functionality for providing clinical support for personalized cancer therapy. However, while a single machine is illustrated, the term “system” shall also be taken to include any collection of machines that individually or jointly execute a set (or multiple sets) of instructions to perform any one or more of the methodologies discussed herein.

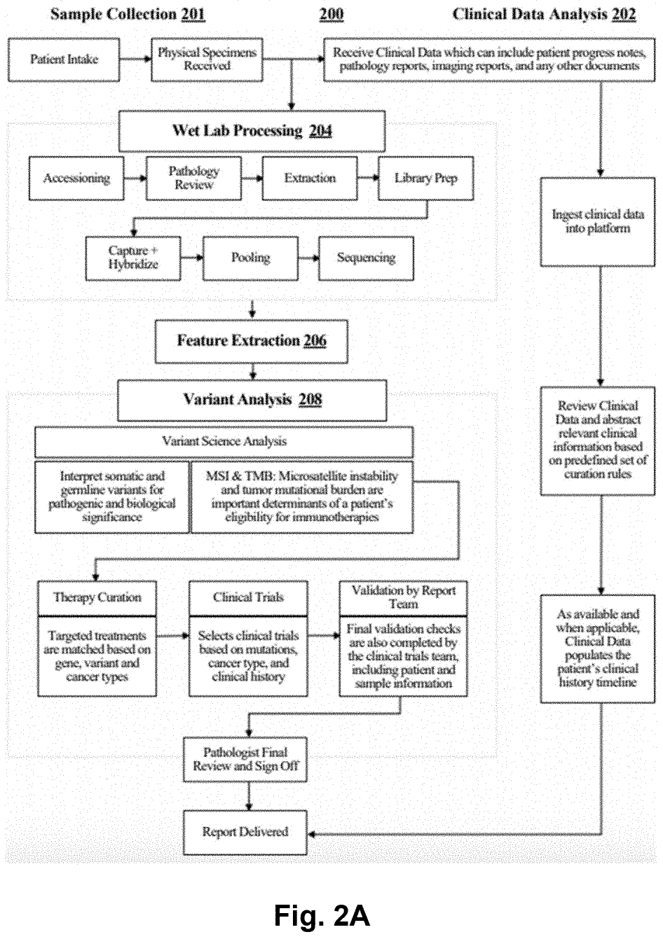

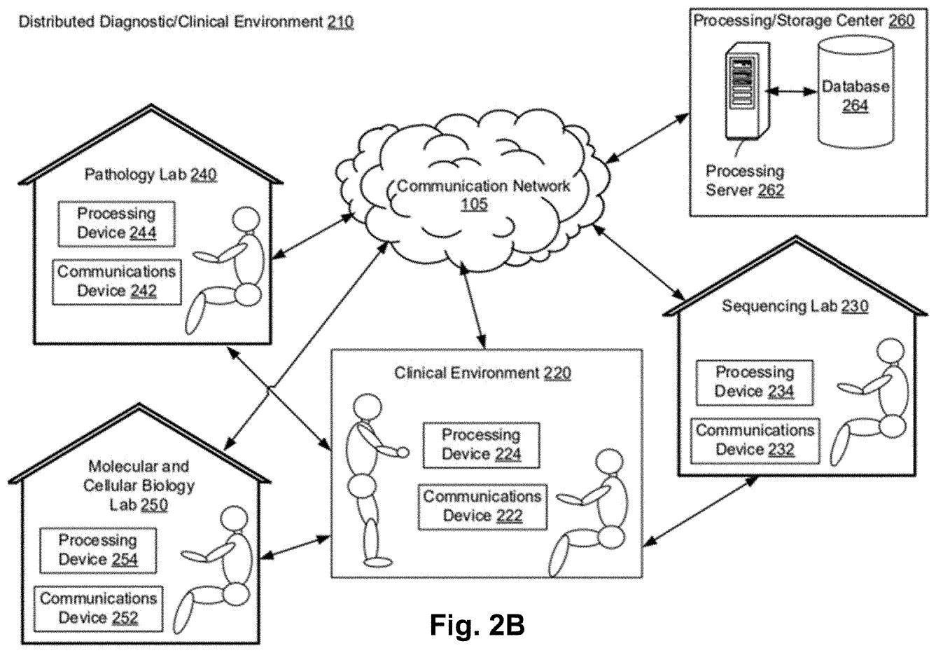

For example, in some embodiments, system 100 includes one or more computers. In some embodiments, the functionality for providing clinical support for personalized cancer or other disease therapy is spread across any number of networked computers and/or resides on each of several networked computers and/or is hosted on one or more virtual machines at a remote location accessible across the communications network 105. For example, different portions of the various modules and data stores illustrated in FIGS. 1A-1B can be stored and/or executed on the various instances of a processing device and/or processing server/database in the distributed diagnostic environment 210 illustrated in FIG. 2B (for example, processing devices 224, 234, 244, and 254, processing server 262, and database 264).

The system may operate in the capacity of a server or a client machine in client-server network environment, as a peer machine in a peer-to-peer (or distributed) network environment, or as a server or a client machine in a cloud computing infrastructure or environment. The system may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine.

In another implementation, the system comprises a virtual machine that includes a module for executing instructions for performing any one or more of the methodologies disclosed herein. In computing, a virtual machine (VM) is an emulation of a computer system that is based on computer architectures and provides functionality of a physical computer. Some such implementations may involve specialized hardware, software, or a combination of hardware and software.

One of skill in the art will appreciate that any of a wide array of different computer topologies are used for the application and all such topologies are within the scope of the present disclosure.

Referring to FIG. 1B, in some embodiments, the system (for example, system 100) includes a patient data store 120 that stores data for patients 121-1 to 121-M (for example, cancer patients or patients being tested for cancer) including one or more sequencing data 122, feature data 125, and clinical assessments 139. These data are used and/or generated by the various processes stored in the bioinformatics module 140 and feature analysis module 160 of system 100, to ultimately generate a report providing clinical support for personalized cancer therapy of a patient. While the feature scope of patient data 121 across all patients may be informationally dense, an individual patient's feature set may be sparsely populated across the entirety of the collective feature scope of all features across all patients. That is to say, the data stored for one patient may include a different set of features that the data stored for another patient. Further, while illustrated as a single data construct in FIG. 1B, different sets of patient data may be stored in different databases or modules spread across one or more system memories.

In some embodiments, sequencing data 122 from one or more sequencing reactions 122-i, including a plurality of sequence reads 123-i-1 to 123-i-K, is stored in the test patient data store 120. The data store may include different sets of sequencing data from a single subject, corresponding to different samples from the patient, for example, a tumor sample, liquid biopsy sample, tumor organoid derived from a patient tumor, and/or a normal sample, and/or to samples acquired at different times, for example, while monitoring the progression, regression, remission, and/or recurrence of a cancer in a subject. The sequence reads may be in any suitable file format, for example, BCL, FASTA, FASTQ, etc. In some embodiments, sequencing data 122 is accessed by a sequencing data processing module 141, which performs various pre-processing, genome alignment, and demultiplexing operations, as described in detail below with reference to bioinformatics module 140. In some embodiments, sequence data that has been aligned to a reference construct, for example, BAM file 124, is stored in test patient data store 120.

In some embodiments, the test patient data store 120 includes feature data 125, for example, that is useful for identifying clinical support for personalized cancer therapy. In some embodiments, the feature data 125 includes personal characteristics 126 of the patient, such as patient name, date of birth, gender, ethnicity, physical address, smoking status, alcohol consumption characteristic, anthropomorphic data, etc.

In some embodiments, the feature data 125 includes medical history data 127 for the patient, such as cancer diagnosis information (for example, date of initial diagnosis, date of metastatic diagnosis, cancer staging, tumor characterization, tissue of origin, previous treatments and outcomes, adverse effects of therapy, therapy group history, clinical trial history, previous and current medications, surgical history, etc.), previous or current symptoms, previous or current therapies, previous treatment outcomes, previous disease diagnoses, diabetes status, diagnoses of depression, diagnoses of other physical or mental maladies, and family medical history. In some embodiments, the feature data 125 includes clinical features 128, such as pathology data 128-1, medical imaging data 128-2, and tissue culture and/or tissue organoid culture data 128-3.

In some embodiments, yet other clinical features, such as previous laboratory testing results, are stored in the test patient data store 120. Medical history data 127 and clinical features may be collected from various sources, including at intake directly from the patient, from an electronic medical record (EMR) or electronic health record (EHR) for the patient, or curated from other sources, such as fields from various testing records (for example, genetic sequencing reports).

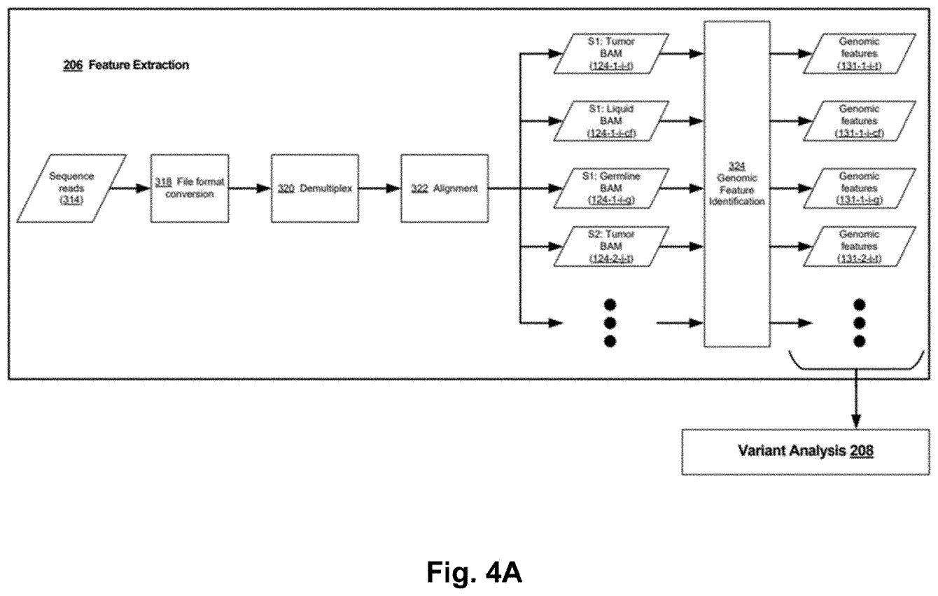

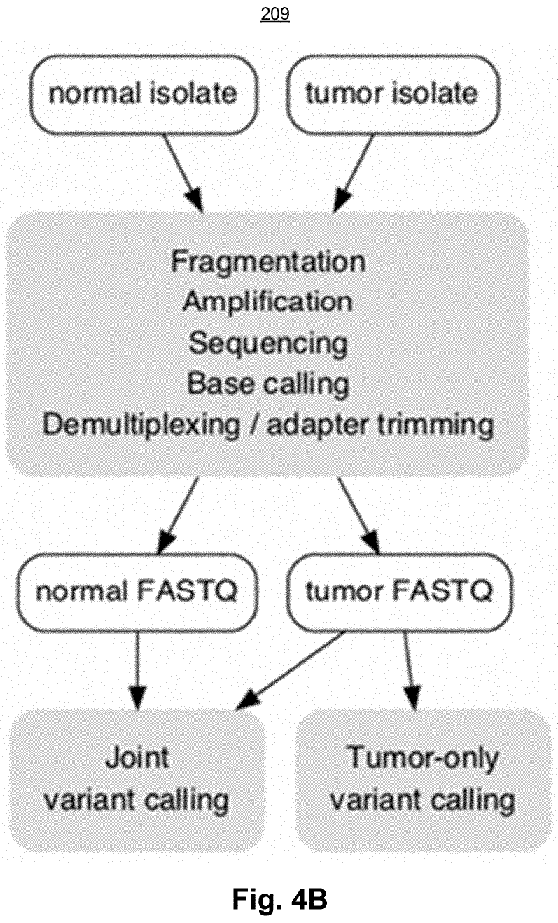

In some embodiments, the feature data 125 includes genomic features 131 for the patient. Non-limiting examples of genomic features include allelic states 132 (for example, the identity of alleles at one or more loci, support for wild type or variant alleles at one or more loci, support for SNVs/MNVs at one or more loci, support for indels at one or more loci, and/or support for gene rearrangements at one or more loci), allelic fractions 133 (for example, ratios of variant to reference alleles (or vice versa), methylation states 132 (for example, a distribution of methylation patterns at one or more loci and/or support for aberrant methylation patterns at one or more loci), genomic copy numbers 135 (for example, a copy number value at one or more loci and/or support for an aberrant (increased or decreased) copy number at one or more loci), tumor mutational burden 136 (for example, a measure of the number of mutations in the cancer genome of the subject), and microsatellite instability status 137 (for example, a measure of the repeated unit length at one or more microsatellite loci and/or a classification of the MSI status for the patient's cancer). In some embodiments, one or more of the genomic features 131 are determined by a nucleic acid bioinformatics pipeline, for example, as described in detail below with reference to FIG. 4. In particular, in some embodiments, the feature data 125 include fusion data, as determined using the improved methods for fusion candidate ranking, as described in further detail below. In some embodiments, one or more of the genomic features 131 are obtained from an external testing source, for example, not connected to the bioinformatics pipeline as described below.

In some embodiments, the feature data 125 further includes data 138 from other -omics fields of study. Non-limiting examples of -omics fields of study that may yield feature data useful for providing clinical support for personalized cancer therapy include transcriptomics, epigenomics, proteomics, metabolomics, metabonomics, microbiomics, lipodomics, glycomics, cellomics, and organoidomics.

In some embodiments, yet other features may include features derived from machine learning approaches, for example, based at least in part on evaluation of any relevant molecular or clinical features, considered alone or in combination, not limited to those listed above. For instance, in some embodiments, one or more latent features learned from evaluation of cancer patient training datasets improve the diagnostic and prognostic power of the various analysis algorithms in the feature analysis module 160.

The skilled artisan will know of other types of features useful for providing clinical support for personalized cancer therapy. The listing of features above is merely representative and should not be construed to be limiting.

In some embodiments, a test patient data store 120 includes clinical assessment data 139 for patients, for example, based off the feature data 125 collected for the subject. In some embodiments, the clinical assessment data 139 includes a catalogue of actionable variants and characteristics 139-1 (for example, genomic alterations and compound metrics based on genomic features known or believed to be targetable by one or more specific cancer therapies), matched therapies 139-2 (for example, the therapies known or believed to be particularly beneficial for treatment of subjects having actionable variants), and/or clinical reports 139-3 generated for the subject, for example, based on identified actionable variants and characteristics 139-1 and/or matched therapies 139-2.

In some embodiments, clinical assessment data 139 is generated by analysis of feature data 125 using the various algorithms of feature analysis module 160, as described in further detail below. In some embodiments, clinical assessment data 139 is generated, modified, and/or validated by evaluation of feature data 125 by a clinician, for example, an oncologist. For instance, in some embodiments, a clinician (for example, at clinical environment 220) uses feature analysis module 160, or accesses test patient data store 120 directly, to evaluate feature data 125 to make recommendations for personalized cancer treatment of a patient. Similarly, in some embodiments, a clinician (for example, at clinical environment 220) reviews recommendations determined using feature analysis module 160 and approves, rejects, or modifies the recommendations, for example, prior to the recommendations being sent to a medical professional treating the cancer patient.

Genetic Sequence Data Generation

Specimen Information

The tumor sample may be a blood sample or tissue sample containing cancer cells or circulating tumor DNA (ctDNA).

For example, a physician may perform a tumor biopsy of a patient by removing a small amount of tumor tissue/specimen from the patient and sending this specimen to a laboratory. The lab may prepare slides from the specimen using slide preparation techniques such as freezing the specimen and slicing layers, setting the specimen in paraffin and slicing layers, smearing the specimen on a slide, or other methods known to those of ordinary skill.

In some embodiments, two or more samples, slices, and/or slides are obtained from a subject—for example, two or more tissue slices can be taken that are contiguous or substantially contiguous to each other. In some cases, the tissue slices are obtained such that some of the pathology slides prepared from the respective slices are imaged (for example, histopathology slides, hematoxylin and eosin stained slides, immunohistochemistry stained slides, etc.), whereas some of the pathology slides are used for obtaining sequencing information.

In some instances, a tumor organoid sample may be processed instead of a patient tumor sample.

In more detail, germ line (“normal”, non-cancerous) DNA may be extracted from either blood (for example, if a patient has cancer that is not a blood cancer) or saliva (for example, if a patient has blood cancer). Normal blood samples may be collected from patients (for example, in PAXgene Blood DNA Tubes) and saliva samples may be collected from patients (for example, in Oragene DNA Saliva Kits).

Blood cancer samples may be collected from patients (for example, in EDTA collection tubes). Macrodissected or microdissected FFPE tissue sections (which may be mounted on a histopathology slide) from solid tumor samples may be analyzed by pathologists to determine overall tumor amount in the sample and percent tumor cellularity as a ratio of tumor to normal nuclei. For each section, background tissue may be excluded or removed such that the section meets a tumor purity threshold (in one example, at least 20% of the cell nuclei in the section are tumor cell nuclei).

FIG. 2A illustrates an example molecular and clinical data analysis workflow 200 to generate a clinical report. Briefly, the workflow begins with patient intake and sample collection 201, where one or more liquid biopsy samples, one or more tumor biopsy, and one or more normal and/or control tissue samples are collected from the patient (for example, at a clinical environment 220 or home healthcare environment). In some embodiments, personal data 126 corresponding to the patient and a record of the one or more biological samples obtained (for example, patient identifiers, patient clinical data, sample type, sample identifiers, cancer conditions, etc.) are entered into a data analysis platform, for example, test patient data store 120. Accordingly, in some embodiments, the methods disclosed herein include obtaining one or more biological samples from one or more subjects, for example, cancer patients. In some embodiments, the subject is a human, for example, a human cancer patient.

In some embodiments, one or more of the biological samples obtained from the patient are a biological liquid sample, also referred to as a liquid biopsy sample. In some embodiments, one or more of the biological samples obtained from the patient are selected from blood, plasma, serum, urine, vaginal fluid, fluid from a hydrocele (for example, of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (for example, thyroid, breast), etc. In some embodiments, the liquid biopsy sample includes blood and/or saliva. In some embodiments, the liquid biopsy sample is peripheral blood. In some embodiments, blood samples are collected from patients in commercial blood collection containers, for example, using a PAXgene® Blood DNA Tubes. In some embodiments, saliva samples are collected from patients in commercial saliva collection containers, for example, using an Oragene® DNA Saliva Kit.

In some embodiments, the liquid biopsy sample has a volume of from about 1 mL to about 50 mL. For example, in some embodiments, the liquid biopsy sample has a volume of about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, or greater.

Liquid biopsy samples include cell free nucleic acids, including cell-free DNA (cfDNA). As described above, cfDNA isolated from cancer patients includes DNA originating from cancerous cells, also referred to as circulating tumor DNA (ctDNA), cfDNA originating from germline (for example, healthy or non-cancerous) cells, and cfDNA originating from hematopoietic cells (for example, white blood cells). The relative proportions of cancerous and non-cancerous cfDNA present in a liquid biopsy sample varies depending on the characteristics (for example, the type, stage, lineage, genomic profile, etc.) of the patient's cancer. As used herein, the ‘tumor burden’ of the subject refers to the percentage cfDNA that originated from cancerous cells.

As described herein, cfDNA is a particularly useful source of biological data for various implementations of the methods and systems described herein, because it is readily obtained from various body fluids. Advantageously, use of bodily fluids facilitates serial monitoring because of the ease of collection, as these fluids are collectable by non-invasive or minimally-invasive methodologies. This is in contrast to methods that rely upon solid tissue samples, such as biopsies, which often times require invasive surgical procedures. Further, because bodily fluids, such as blood, circulate throughout the body, the cfDNA population represents a sampling of many different tissue types from many different locations.

In some embodiments, a liquid biopsy sample is separated into two different samples. For example in some embodiments, a blood sample is separated into a blood plasma sample, containing cfDNA, and a buffy coat preparation, containing white blood cells.

In some embodiments, a plurality of liquid biopsy samples is obtained from a respective subject at intervals over a period of time (for example, using serial testing). For example, in some such embodiments, the time between obtaining liquid biopsy samples from a respective subject is at least 1 day, at least 2 days, at least 1 week, at least 2 weeks, at least 1 month, at least 2 months, at least 3 months, at least 4 months, at least 6 months, or at least 1 year.

In some embodiments, one or more biological samples collected from the patient is a solid tissue sample, for example, a solid tumor sample or a solid normal tissue sample. Methods for obtaining solid tissue samples, for example, of cancerous and/or normal tissue are known in the art, and are dependent upon the type of tissue being sampled. For example, bone marrow biopsies and isolation of circulating tumor cells can be used to obtain samples of blood cancers, endoscopic biopsies can be used to obtain samples of cancers of the digestive tract, bladder, and lungs, needle biopsies (for example, fine-needle aspiration, core needle aspiration, vacuum-assisted biopsy, and image-guided biopsy, can be used to obtain samples of subdermal tumors, skin biopsies, for example, shave biopsy, punch biopsy, incisional biopsy, and excisional biopsy, can be used to obtain samples of dermal cancers, and surgical biopsies can be used to obtain samples of cancers affecting internal organs of a patient. In some embodiments, a solid tissue sample is a formalin-fixed tissue (FFT). In some embodiments, a solid tissue sample is a macro-dissected formalin fixed paraffin embedded (FFPE) tissue. In some embodiments, a solid tissue sample is a fresh frozen tissue sample.

In some embodiments, a dedicated normal sample is collected from the patient, for co-processing with a liquid biopsy sample. Generally, the normal sample is of a non-cancerous tissue, and can be collected using any tissue collection means described above. In some embodiments, buccal cells collected from the inside of a patient's cheeks are used as a normal sample. Buccal cells can be collected by placing an absorbent material, for example, a swab, in the subjects mouth and rubbing it against their cheek, for example, for at least 15 second or for at least 30 seconds. The swab is then removed from the patient's mouth and inserted into a tube, such that the tip of the tube is submerged into a liquid that serves to extract the buccal cells off of the absorbent material. An example of buccal cell recovery and collection devices is provided in U.S. Pat. No. 9,138,205, the content of which is hereby incorporated by reference, in its entirety, for all purposes. In some embodiments, the buccal swab DNA is used as a source of normal DNA in circulating heme malignancies.

Probe Overview

In some embodiments, a plurality of nucleic acid probes (for example, a probe set) is used to enrich one or more target sequences in a nucleic acid sample (for example, an isolated nucleic acid sample or a nucleic acid sequencing library). Probes may be designed and created in accordance with methods known in the art. In some embodiments, the probe set includes probes targeting one or more gene loci, for example, exon or intron loci. In some embodiments, the probe set includes probes targeting one or more loci not encoding a protein, for example, regulatory loci, miRNA loci, and other non-coding loci, for example, that have been found to be associated with cancer. In some embodiments, the plurality of loci include at least 25, 50, 100, 150, 200, 250, 300, 350, 400, 500, 750, 1000, 2500, 5000, or more human genomic loci.

Generally, probes for enrichment of nucleic acids (for example, complementary DNA, cDNA, generated from nucleic acids extracted or isolated from a biological specimen, including extracted or isolated RNA) include DNA, RNA, or a modified nucleic acid structure with a base sequence that is complementary to a loci of interest. For instance, a probe designed to hybridize to a loci in a cDNA molecule can contain a sequence that is complementary to either strand, because the cDNA molecules may be double stranded. In some embodiments, each probe in the plurality of probes includes a nucleic acid sequence that is identical or complementary to at least 10, at least 11, at least 12, at least 13, at least 14, or at least 15 consecutive bases of a loci of interest. In some embodiments, each probe in the plurality of probes includes a nucleic acid sequence that is identical or complementary to at least 20, 25, 30, 40, 50, 75, 100, 150, 200, or more consecutive bases of a locus of interest.

Probes may be created in accordance with the methods set forth in FastPCR Software for PCR Primer and Probe Design and Repeat Search (Kalendar et al., 2009 Genes, Genomes, and Genomics, 3 (Special Issue 1), pp. 1-14) which is incorporated by reference herein.

Targeted-panels provide several benefits for nucleic acid sequencing. In one example, panels targeting genes with high variability among individual subjects, humans, or even cells within subjects or humans may facilitate bioinformatics processing to determine the sequences of those genes. For example, if a “whole exome” or targeted sequencing panel is not generating a sufficient number of sequencing reads mapping to the high-variable genes, probes targeting the high-variable genes may be added to the whole exome or targeted sequence panel probes to increase the number reads mapping to high-variable genes.

In some embodiments, the gene panel is a whole-exome panel that analyzes the exomes of a biological sample. In some embodiments, the gene panel is a whole-genome panel that analyzes the genome of a specimen. In some preferred embodiments, the gene panel is optimized for use with specific cells or cell types of interest. For instance, the gene panel may be optimized for use in a cancer gene panel (for example, to provide clinical decision support related to cancer treatment).

In some embodiments, the probes include additional nucleic acid sequences that do not share any homology to the loci of interest. For example, in some embodiments, the probes also include nucleic acid sequences containing an identifier sequence, for example, a unique molecular identifier (UMI), for example, which is unique to a particular sample or subject. Examples of identifier sequences are described, for example, in Kivioja et al., 2011, Nat. Methods 9(1), pp. 72-74 and Islam et al., 2014, Nat. Methods 11(2), pp. 163-66, which are incorporated by reference herein. Similarly, in some embodiments, the probes also include primer nucleic acid sequences useful for amplifying the nucleic acid molecule of interest, for example, using PCR. In some embodiments, the probes also include a capture sequence designed to hybridize to an anti-capture sequence for recovering the nucleic acid molecule of interest from the sample.

Likewise, in some embodiments, the probes each include a non-nucleic acid affinity moiety covalently attached to nucleic acid molecule that is complementary to the loci of interest, for recovering the nucleic acid molecule of interest. Non-limited examples of non-nucleic acid affinity moieties include biotin, digoxigenin, and dinitrophenol. In some embodiments, the probe is attached to a solid-state surface or particle, for example, a dip-stick or magnetic bead, for recovering the nucleic acid of interest. In some embodiments, the methods described herein include amplifying the nucleic acids that bound to the probe set prior to further analysis, for example, sequencing. Methods for amplifying nucleic acids, for example, by PCR, are well known in the art.

Probe Concentration

In some embodiments, probes may be included as part of a comprehensive genomic profiling panel. Examples include a whole exome RNAseq panel, a targeted enrichment sequencing panel, a whole-exome panel, a whole genome panel, etc.

Probes may be separated into various pools. The concentration of each group (pool) of probes may be adjusted to achieve desired coverage. The concentration of each pool may be adjusted in accordance with, for example, the systems and methods disclosed in U.S. Prov. Patent App. No. 62/924,073 and U.S. patent application Ser. No. 17/076,704, filed on and published as U.S. Pre-Grant Publication No. 2021/0115511 on Apr. 22, 2021 and incorporated by reference herein in its entirety.

DNA Profiling

In some embodiments, each DNA data set may be generated by processing a cancer sample and a non-cancer sample from the same patient, or only a cancer sample through DNA next generation sequencing (NGS), designed to sequence either the whole exome or a targeted panel of cancer-related genes, to generate DNA sequencing data. The cancer sample may be tissue, blood, or cell-free, circulating tumor DNA. The DNA sequencing data may be processed by a bioinformatics pipeline to generate a DNA variant call file (among other outputs) for each sample.

The biological samples collected from the patient are, optionally, sent to various analytical environments (for example, sequencing lab 230, pathology lab 240, and/or molecular and cellular biology lab 250) for processing (for example, data collection) and/or analysis (for example, feature extraction). Wet lab processing 204 may include the steps of cataloguing samples (for example, accessioning), examining clinical features of one or more samples (for example, pathology review), and nucleic acid sequence analysis (for example, extraction, library prep, capture+hybridize, pooling, and sequencing). In some embodiments, the workflow includes clinical analysis of one or more biological samples collected from the subject, for example, at a pathology lab 240 and/or a molecular and cellular biology lab 250, to generate clinical features such as pathology features 128-3, imaging data 128-3, and/or tissue culture/organoid data 128-3.

In some embodiments, the pathology data 128-1 collected during clinical evaluation includes visual features identified by a pathologist's inspection of a specimen (for example, a solid tumor biopsy), for example, of stained H&E or IHC slides. In some embodiments, the sample is a solid tissue biopsy sample. In some embodiments, the tissue biopsy sample is a formalin-fixed tissue (FFT), for example, a formalin-fixed paraffin-embedded (FFPE) tissue. In some embodiments, the tissue biopsy sample is an FFPE or FFT block. In some embodiments, the tissue biopsy sample is a fresh-frozen tissue biopsy. The tissue biopsy sample can be prepared in thin sections (for example, by cutting and/or affixing to a slide), to facilitate pathology review (for example, by staining with immunohistochemistry stain for IHC review and/or with hematoxylin and eosin stain for H&E pathology review). For instance, analysis of slides for H&E staining or IHC staining may reveal features such as tumor infiltration, programmed death-ligand 1 (PD-L1) status, human leukocyte antigen (HLA) status, or other immunological features.

In some embodiments, a liquid sample (for example, blood) collected from the patient (for example, in EDTA-containing collection tubes) is prepared on a slide (for example, by smearing) for pathology review. In some embodiments, macrodissected FFPE tissue sections, which may be mounted on a histopathology slide, from solid tissue samples (for example, tumor or normal tissue) are analyzed by pathologists. In some embodiments, tumor samples are evaluated to determine, for example, the tumor purity of the sample, the percent tumor cellularity as a ratio of tumor to normal nuclei, etc. For each section, background tissue may be excluded or removed such that the section meets a tumor purity threshold, for example, where at least 20% of the nuclei in the section are tumor nuclei, or where at least 25%, 30%, 40%, 50%, 60%, 70%, 80%, 90%, or more of the nuclei in the section are tumor nuclei.

In some embodiments, pathology data 128-1 is extracted, in addition to or instead of visual inspection, using computational approaches to digital pathology, for example, providing morphometric features extracted from digital images of stained tissue samples. A review of digital pathology methods is provided in Bera, K. et al., Nat. Rev. Clin. Oncol., 16:703-15 (2019), the content of which is hereby incorporated by reference, in its entirety, for all purposes. In some embodiments, pathology data 128-1 includes features determined using machine learning algorithms to evaluate pathology data collected as described above.

In some embodiments, imaging data 128-2 collected during clinical evaluation includes features identified by review of in-vitro and/or in-vivo imaging results (for example, of a tumor site), for example a size of a tumor, tumor size differentials over time (such as during treatment or during other periods of change). In some embodiments, imaging data 128-2 includes features determined using machine learning algorithms to evaluate imaging data collected as described above.

In some embodiments, tissue culture/organoid data 128-3 collected during clinical evaluation includes features identified by evaluation of cultured tissue from the subject. For instance, in some embodiments, tissue samples obtained from the patients (for example, tumor tissue, normal tissue, or both) are cultured (for example, in liquid culture, solid-phase culture, and/or organoid culture) and various features, such as cell morphology, growth characteristics, genomic alterations, and/or drug sensitivity, are evaluated. In some embodiments, tissue culture/organoid data 128-3 includes features determined using machine learning algorithms to evaluate tissue culture/organoid data collected as described above. Examples of tissue organoid (for example, personal tumor organoid) culturing and feature extractions thereof are described in U.S. Provisional Application Serial No. 62/924,621, filed on Oct. 22, 2019, and U.S. patent application Ser. No. 16/693,117, filed on Nov. 22, 2019, the contents of which are hereby incorporated by reference, in their entireties, for all purposes.

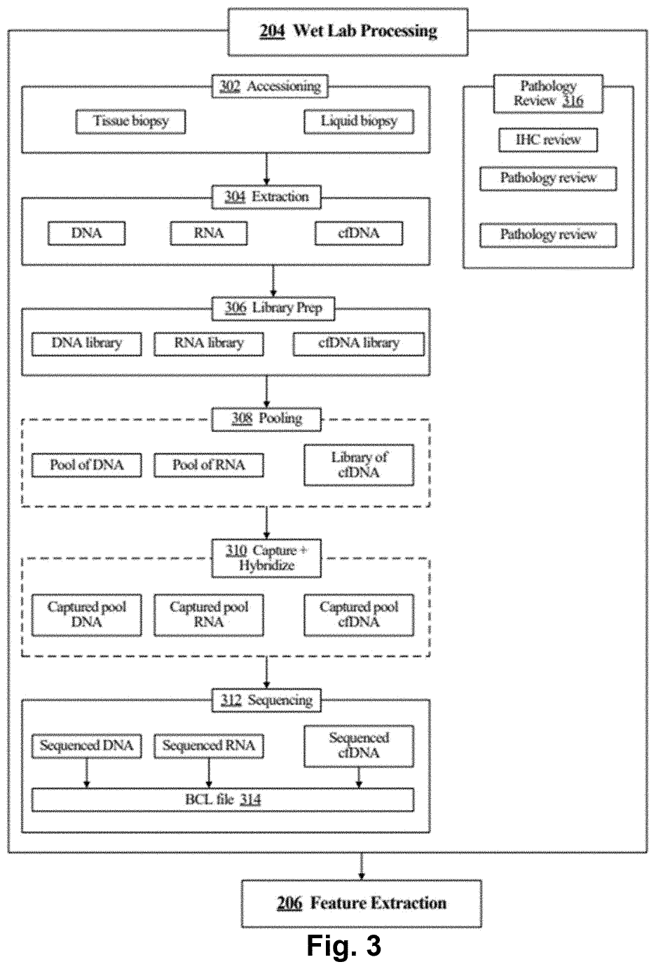

Nucleic acid sequencing of one or more samples collected from the subject is performed, for example, at sequencing lab 230, during wet lab processing 204. An example workflow for nucleic acid sequencing is illustrated in FIG. 3. In some embodiments, the one or more biological samples obtained at the sequencing lab 230 are accessioned (302), to track the sample and data through the sequencing process.

Next, nucleic acids, for example, RNA and/or DNA are extracted (304) from the one or more biological samples. Methods for isolating nucleic acids from biological samples are known in the art, and are dependent upon the type of nucleic acid being isolated (for example, cfDNA, DNA, and/or RNA) and the type of sample from which the nucleic acids are being isolated (for example, liquid biopsy samples, white blood cell buffy coat preparations, formalin-fixed paraffin-embedded (FFPE) solid tissue samples, and fresh frozen solid tissue samples). The selection of any particular nucleic acid isolation technique for use in conjunction with the embodiments described herein is well within the skill of the person having ordinary skill in the art, who will consider the sample type, the state of the sample, the type of nucleic acid being sequenced and the sequencing technology being used.

For instance, many techniques for DNA isolation, for example, genomic DNA isolation, from a tissue sample are known in the art, such as organic extraction, silica adsorption, and anion exchange chromatography. Likewise, many techniques for RNA isolation, for example, mRNA isolation, from a tissue sample are known in the art. For example, acid guanidinium thiocyanate-phenol-chloroform extraction (see, for example, Chomczynski and Sacchi, 2006, Nat Protoc, 1(2):581-85, which is hereby incorporated by reference herein), and silica bead/glass fiber adsorption (see, for example, Poeckh, T. et al., 2008, Anal Biochem., 373(2):253-62, which is hereby incorporated by reference herein). The selection of any particular DNA or RNA isolation technique for use in conjunction with the embodiments described herein is well within the skill of the person having ordinary skill in the art, who will consider the tissue type, the state of the tissue, for example, fresh, frozen, formalin-fixed, paraffin-embedded (FFPE), and the type of nucleic acid analysis that is to be performed.

In some embodiments where the biological sample is a liquid biopsy sample, for example, a blood or blood plasma sample, cfDNA is isolated from blood samples using commercially available reagents, including proteinase K, to generate a liquid solution of cfDNA.

Additionally, DNA may be isolated from cells in blood samples, saliva samples, and tissue sections by lysing cells and using commercially available reagents, including proteinase K to generate a liquid solution of DNA.

In some embodiments, isolated DNA or cfDNA molecules are mechanically sheared to an average length using an ultrasonicator (for example, a Covaris ultrasonicator). In some embodiments, isolated nucleic acid molecules are analyzed to determine their fragment size, for example, through gel electrophoresis techniques and/or the use of a device such as a LabChip GX Touch.

In some embodiments, quality control testing is performed on the extracted nucleic acids (for example, DNA and/or RNA), for example, to assess the nucleic acid concentration and/or fragment size. For example, sizing of DNA fragments provides valuable information used for downstream processing, such as determining whether DNA fragments require additional shearing prior to sequencing.

Wet lab processing then includes preparing a nucleic acid library from the isolated nucleic acids (for example, cfDNA, DNA, and/or RNA). For example, in some embodiments, DNA libraries (for example, gDNA and/or cfDNA libraries) are prepared from isolated DNA from the one or more biological samples. In some embodiments, the DNA libraries are prepared using a commercial library preparation kit, for example, the KAPA Hyper Prep Kit, a New England Biolabs (NEB) kit, or a similar kit.

In some embodiments, during library preparation, adapters (for example, UDI adapters, such as Roche SeqCap dual end adapters, or UMI adapters such as full length or stubby Y adapters) are ligated onto the nucleic acid molecules. In some embodiments, the adapters include unique molecular identifiers (UMIs), which are short nucleic acid sequences (for example, 4-10 base pairs) that are added to ends of DNA fragments during adapter ligation. In some embodiments, UMIs are degenerate base pairs that serve as a unique tag that can be used to identify sequence reads originating from a specific DNA fragment. In some embodiments, for example, when multiplex sequencing will be used to sequence DNA from a plurality of samples (for example, from the same or different subjects) in a single sequencing reaction, a patient-specific index is also added to the nucleic acid molecules. In some embodiments, the patient specific index is a short nucleic acid sequence (for example, 3-20 nucleotides) that are added to ends of DNA fragments during library construction, that serve as a unique tag that can be used to identify sequence reads originating from a specific patient sample. Examples of identifier sequences are described, for example, in Kivioja et al., Nat. Methods 9(1):72-74 (2011) and Islam et al., Nat. Methods 11(2):163-66 (2014), the contents of which are hereby incorporated by reference, in their entireties, for all purposes.

In some embodiments, an adapter includes a PCR primer landing site, designed for efficient binding of a PCR or second-strand synthesis primer used during the sequencing reaction. In some embodiments, an adapter includes an anchor binding site, to facilitate binding of the DNA molecule to anchor oligonucleotide molecules on a sequencer flow cell, serving as a seed for the sequencing process by providing a starting point for the sequencing reaction. During PCR amplification following adapter ligation, the UMIs, patient indexes, and binding sites are replicated along with the attached DNA fragment. This provides a way to identify sequence reads that came from the same original fragment in downstream analysis.

In some embodiments, DNA libraries are amplified and purified using commercial reagents, (for example, Axygen MAG PCR clean up beads). In some such embodiments, the concentration and/or quantity of the DNA molecules are then quantified using a fluorescent dye and a fluorescence microplate reader, standard spectrofluorometer, or filter fluorometer. In some embodiments, library amplification is performed on a device (for example, an Illumina C-Bot2) and the resulting flow cell containing amplified target-captured DNA libraries is sequenced on a next generation sequencer (for example, an Illumina HiSeq 4000 or an Illumina NovaSeq 6000) to a unique on-target depth selected by the user. In some embodiments, DNA library preparation is performed with an automated system, using a liquid handling robot (for example, a SciClone NGSx).

In some embodiments, wet lab processing 204 includes pooling (308) DNA molecules from a plurality of libraries, corresponding to different samples from the same and/or different patients, to forming a sequencing pool of DNA libraries. When the pool of DNA libraries is sequenced, the resulting sequence reads correspond to nucleic acids isolated from multiple samples. The sequence reads can be separated into different sequence read files, corresponding to the various samples represented in the sequencing read based on the unique identifiers present in the added nucleic acid fragments. In this fashion, a single sequencing reaction can generate sequence reads from multiple samples. Advantageously, this allows for the processing of more samples per sequencing reaction.

In some embodiments, wet lab processing 204 includes enriching (310) a sequencing library, or pool of sequencing libraries, for target nucleic acids, for example, nucleic acids encompassing loci that are informative for precision oncology and/or used as internal controls for the sequencing or bioinformatics processes. In some embodiments, enrichment is achieved by hybridizing target nucleic acids in the sequencing library to probes that hybridize to the target sequences, and then isolating the captured nucleic acids away from off-target nucleic acids that are not bound by the capture probes.

Advantageously, enriching for target sequences prior to sequencing nucleic acids significantly reduces the costs and time associated with sequencing, facilitates multiplex sequencing by allowing multiple samples to be mixed together for a single sequencing reaction, and significantly reduces the computation burden of aligning the resulting sequence reads, as a result of significantly reducing the total amount of nucleic acids analyzed from each sample.

In some embodiments, the enrichment is performed prior to pooling multiple nucleic acid sequencing libraries. However, in other embodiments, the enrichment is performed after pooling nucleic acid sequencing libraries, which has the advantage of reducing the number of enrichment assays that have to be performed.

In some embodiments, the enrichment is performed prior to generating a nucleic acid sequencing library. This has the advantage that fewer reagents are needed to perform both the enrichment (because there are fewer target sequences at this point, prior to library amplification) and the library production (because there are fewer nucleic acid molecules to tag and amplify after the enrichment). However, this raises the possibility of pull-down bias and/or that small variations in the enrichment protocol will result in less consistent results.

In some embodiments, nucleic acid libraries are pooled (two or more DNA libraries may be mixed to create a pool) and treated with reagents to reduce off-target capture, for example Human COT-1 and/or IDT xGen Universal Blockers. Pools may be dried in a vacufuge and resuspended. DNA libraries or pools may be hybridized to a probe set (for example, a probe set specific to a panel that includes loci from at least 100, 600, 1,000, 10,000, etc. of the 19,000 known human genes) and amplified with commercially available reagents (for example, the KAPA HiFi HotStart ReadyMix). For example, in some embodiments, a pool is incubated in an incubator, PCR machine, water bath, or other temperature-modulating device to allow probes to hybridize. Pools may then be mixed with Streptavidin-coated beads or another means for capturing hybridized DNA-probe molecules, such as DNA molecules representing exons of the human genome and/or genes selected for a genetic panel.

Pools may be amplified and purified more than once using commercially available reagents, for example, the KAPA HiFi Library Amplification kit and Axygen MAG PCR clean up beads, respectively. The pools or DNA libraries may be analyzed to determine the concentration or quantity of DNA molecules, for example by using a fluorescent dye (for example, PicoGreen pool quantification) and a fluorescence microplate reader, standard spectrofluorometer, or filter fluorometer. In one example, the DNA library preparation and/or capture steps may be performed with an automated system, using a liquid handling robot (for example, a SciClone NGSx).

Sequence reads are then generated (312) from the sequencing library or pool of sequencing libraries. Sequencing data may be acquired by any methodology known in the art. For example, next generation sequencing (NGS) techniques such as sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators. In some embodiments, sequencing is performed using next generation sequencing technologies, such as short-read technologies. In other embodiments, long-read sequencing or another sequencing method known in the art is used.

Next-generation sequencing produces millions of short reads (for example, sequence reads) for each biological sample. Accordingly, in some embodiments, the plurality of sequence reads obtained by next-generation sequencing of cfDNA molecules are DNA sequence reads. In some embodiments, the sequence reads have an average length of at least fifty nucleotides. In other embodiments, the sequence reads have an average length of at least 50, 60, 70, 80, 90, 100, 150, 200, 250, 300, or more nucleotides.

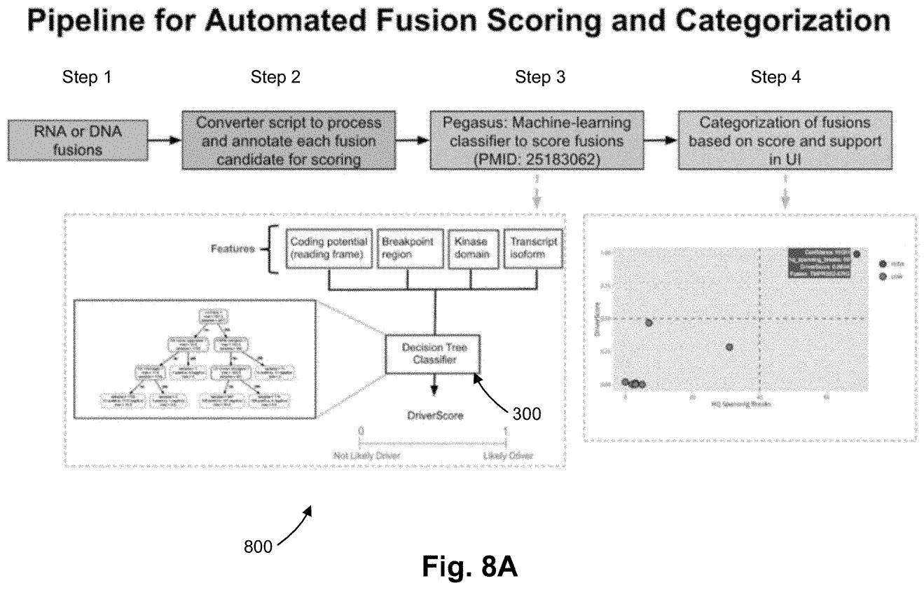

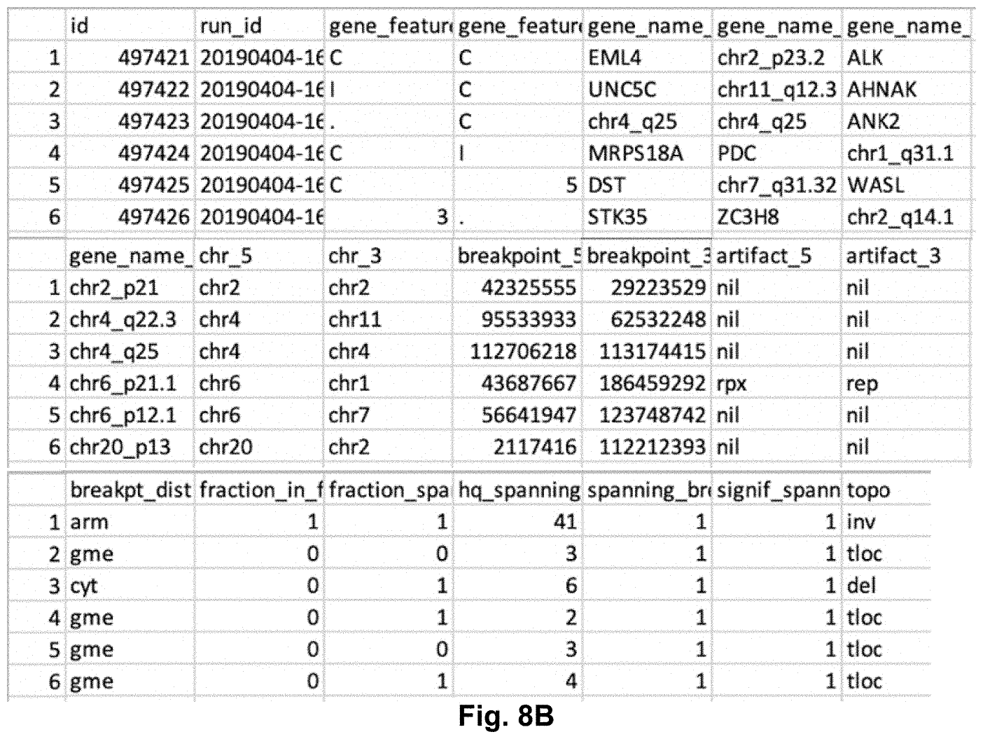

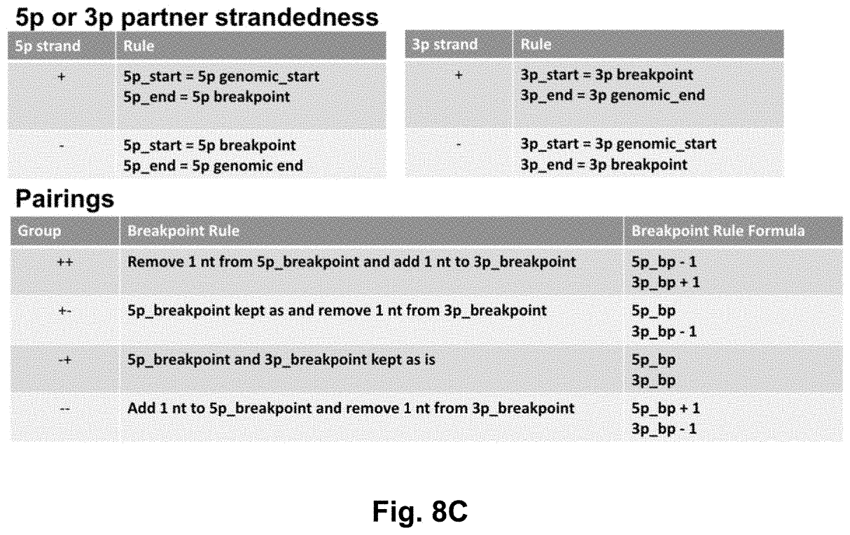

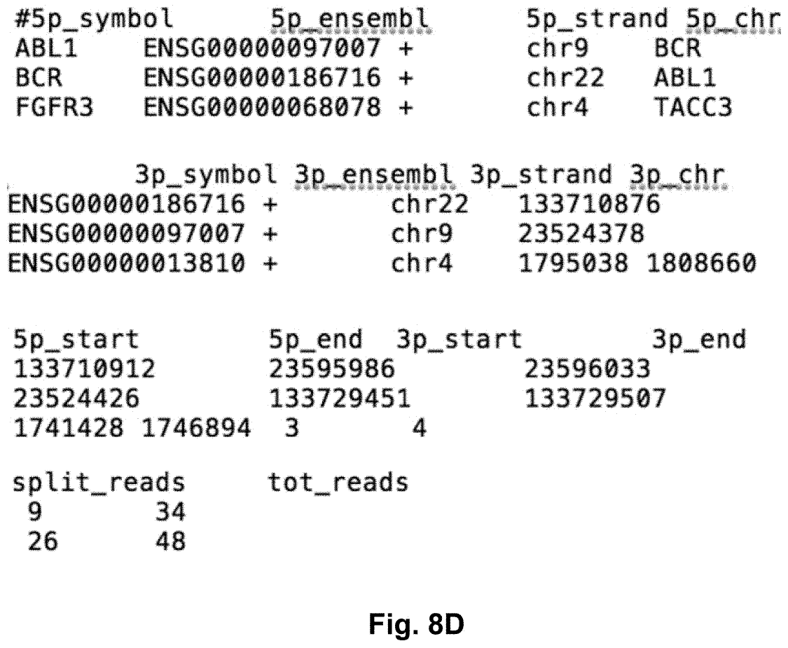

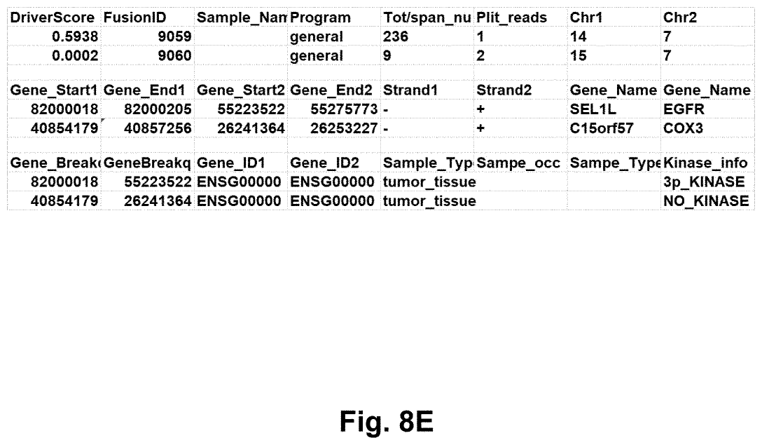

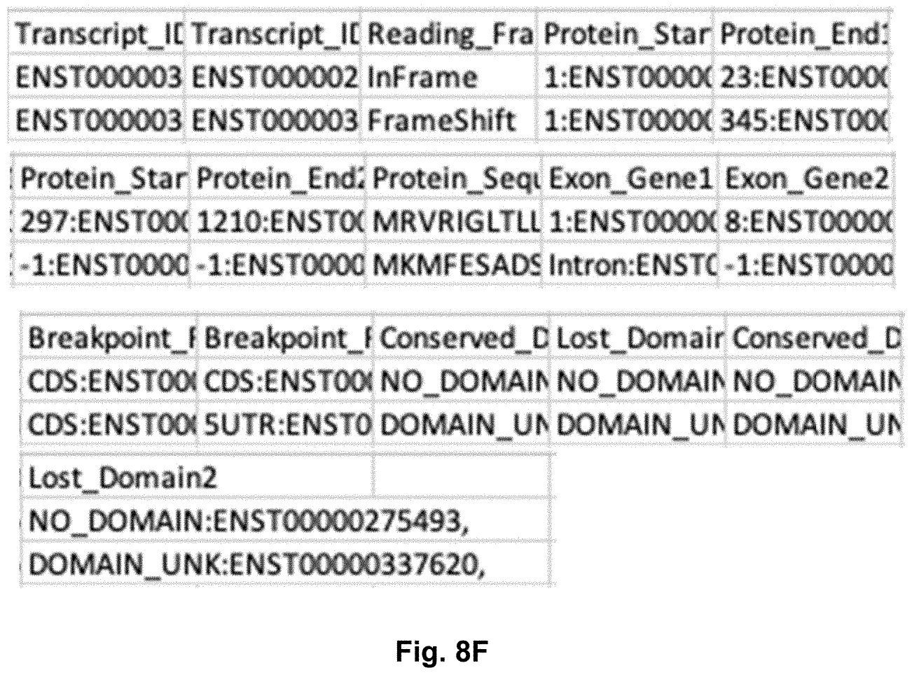

In some embodiments, sequencing is performed after enriching for nucleic acids (for example, cfDNA, gDNA, and/or RNA) encompassing a plurality of predetermined target sequences, for example, human genes and/or non-coding sequences associated with cancer. Advantageously, sequencing a nucleic acid sample that has been enriched for target nucleic acids, rather than all nucleic acids isolated from a biological sample, significantly reduces the average time and cost of the sequencing reaction. Accordingly, in some preferred embodiments, the methods described herein include obtaining a plurality of sequence reads of nucleic acids that have been hybridized to a probe set for hybrid-capture enrichment (for example, of one or more genes listed in FIG. 8B).

In some embodiments, panel-targeting sequencing is performed to an average on-target depth of at least 500×, at least 750×, at least 1000×, at least 2500×, at least 500×, at least 10,000×, or greater depth. In some embodiments, samples are further assessed for uniformity above a sequencing depth threshold (for example, 95% of all targeted base pairs at 300× sequencing depth). In some embodiments, the sequencing depth threshold is a minimum depth selected by a user or practitioner.

In some embodiments, the sequence reads are obtained by a whole genome or whole exome sequencing methodology. In some such embodiments, whole exome capture steps may be performed with an automated system, using a liquid handling robot (for example, a SciClone NGSx). Whole genome sequencing, and to some extent whole exome sequencing, is typically performed at lower sequencing depth than smaller target-panel sequencing reactions, because many more loci are being sequenced. For example, in some embodiments, whole genome or whole exome sequencing is performed to an average sequencing depth of at least 3×, at least 5×, at least 10×, at least 15×, at least 20×, or greater. In some embodiments, low-pass whole genome sequencing (LPWGS) techniques are used for whole genome or whole exome sequencing. LPWGS is typically performed to an average sequencing depth of about 0.25% to about 5%, more typically to an average sequencing depth of about 0.5× to about 3×.

Because of the differences in the sequencing methodologies, data obtained from targeted-panel sequencing is better suited for certain analyses than data obtained from whole genome/whole exome sequencing, and vice versa. For instance, because of the higher sequencing depth achieved by targeted-panel sequencing, the resulting sequence data is better suited for the identification of variant alleles present at low allelic fractions in the sample, for example, less than 20%. By contrast, data generated from whole genome/whole exome sequencing is better suited for the estimation of genome-wide metrics, such as tumor mutational burden, because the entire genome is better represented in the sequencing data. Accordingly, in some embodiments, a nucleic acid sample, for example, a cfDNA, gDNA, or mRNA sample, is evaluated using both targeted-panel sequencing and whole genome/whole exome sequencing (for example, LPWGS).

In some embodiments, the raw sequence reads resulting from the sequencing reaction are output from the sequencer in a native file format, for example, a BCL file. In some embodiments, the native file is passed directly to a bioinformatics pipeline (for example, variant analysis pipeline 208), components of which are described in detail below. In other embodiments, one or more pre-processing steps are performed prior to passing the sequences to the bioinformatics platform. For instance, in some embodiments, the format of the sequence read file is converted from the native file format (for example, BCL) to a file format compatible with one or more algorithms used in the bioinformatics pipeline (for example, FASTQ or FASTA). In some embodiments, the raw sequence reads are filtered to remove sequences that do not meet one or more quality thresholds. In some embodiments, raw sequence reads generated from the same unique nucleic acid molecule in the sequencing read are collapsed into a single sequence read representing the molecule, for example, using UMIs as described above. In some embodiments, one or more of these pre-processing steps are performed within the bioinformatics pipeline itself.

In one example, a sequencer may generate a BCL file. A BCL file may include raw image data of a plurality of patient specimens which are sequenced. BCL image data is an image of the flow cell across each cycle during sequencing. A cycle may be implemented by illuminating a patient specimen with a specific wavelength of electromagnetic radiation, generating a plurality of images which may be processed into base calls via BCL to FASTQ processing algorithms which identify which base pairs are present at each cycle. The resulting FASTQ file includes the entirety of reads for each patient specimen paired with a quality metric, for example, in a range from 0 to 64 where a 64 is the best quality and a 0 is the worst quality. In embodiments where both a liquid biopsy sample and a normal tissue sample are sequenced, sequence reads in the corresponding FASTQ files may be matched, such that a liquid biopsy-normal analysis may be performed.

FASTQ format is a text-based format for storing both a biological sequence, such as nucleotide sequence, and its corresponding quality scores. These FASTQ files are analyzed to determine what genetic variants or copy number changes are present in the sample. Each FASTQ file contains reads that may be paired-end or single reads, and may be short-reads or long-reads, where each read represents one detected sequence of nucleotides in a nucleic acid molecule that was isolated from the patient sample or a copy of the nucleic acid molecule, detected by the sequencer. Each read in the FASTQ file is also associated with a quality rating. The quality rating may reflect the likelihood that an error occurred during the sequencing procedure that affected the associated read. In some embodiments, the results of paired-end sequencing of each isolated nucleic acid sample are contained in a split pair of FASTQ files, for efficiency. Thus, in some embodiments, forward (Read 1) and reverse (Read 2) sequences of each isolated nucleic acid sample are stored separately but in the same order and under the same identifier.

RNA profiling may be achieved, for instance, using the following methods. Transcriptome analysis, the study of the complete set of RNA transcripts that are produced by a cell (the transcriptome), offers a promising means to identify genetic variants that are correlated with disease state and disease progression. For example, to identify genetic variants that are associated with cancer, transcriptome analysis may be performed on a sample collected from a patient that contains cancer cells. Suitable patient samples include tissue samples, tumors (for example, a solid tumor), biopsies, and bodily fluids (for example, blood, serum, plasma, sputum, lavage fluid, cerebrospinal fluid, urine, semen, sweat, tears, saliva). Alternatively, transcriptome analysis may be performed on an organoid that was generated from a human cancer specimen (a “tumor organoid”). Sequencing may be performed on a single cell specimen or on a multi-cell specimen. While RNA sequencing (RNA-seq) can be performed on any patient sample that contains RNA, those of skill in the art will appreciate that the sequencing protocol should be tailored to the particular sample in use. For instance, RNA tends to be highly degraded in tissue samples that have been processed for histology (for example, formalin fixed, paraffin embedded (FFPE) tissue sections). Accordingly, investigators will modify several key steps in the RNA-seq protocol to mitigate sequencing artifacts (see, for example, BMC Medical Genomics 12, 195 (2019)). Today, transcriptome analysis is predominantly performed using high-throughput RNA sequencing (RNA-Seq), which detects the RNA transcripts in a sample using a next-generation sequencer. The first step in performing RNA-seq is to extract RNA from the sample.

The first step in extracting RNA from a sample is often to lyse the cells present in that sample. Several physical disruption methods are commonly used to lyse cells, including, for example, mechanical disruption (for example, using a blender or tissue homogenizer), liquid homogenization (for example, using a dounce or French press), high frequency sound waves (for example, using a sonicator), freeze/thaw cycles, heating, manual grinding (for example, using a mortar and pestle), and bead-beating (for example, using a Mini-beadbeater-96 from BioSpec). Cells are also commonly lysed using reagents that contain a detergent, many of which are commercially available (for example, QIAzol Lysis Reagent from QIAGEN, FastBreak™ Cell Lysis Reagent from Promega). Often, physical disruption methods are performed in a “homogenization buffer” that contains, for example, lysis reagents such as detergents or proteases (for example, proteinase K) that increase the efficiency of lysis. Homogenization buffers may also include anti-foaming agents and/or RNase inhibitors to protect RNA from degradation. Those of skill in the art will appreciate that different cell lysis techniques may be required to obtain the best possible yield from different tissues. Techniques that minimize the degradation of the released RNA and that avoid the release of nuclear chromatin are preferred.

After the cells have been lysed, RNA can be separated from other cellular components. Total RNA is commonly isolated using guanidinium thiocyanate-phenol-chloroform extraction (for example, using TRIzol) or by performing trichloroacetic acid/acetone precipitation followed by phenol extraction. However, there are also many commercially available column-based systems for extracting RNA (for example, PureLink RNA Mini Kit by Invitrogen and Direct-zol Miniprep kit by Zymo Research). Ideally, the isolated RNA will contain very little DNA and enzymatic contamination. To this end, the isolation method may utilize agents that eliminate DNA (for example, TURBO DNase-I), and/or remove enzymatic proteins from the sample (for example, Agencourt® RNAClean® XP beads from Beckman Coulter). In some cases, whole transcriptome sequencing is used to analyze all of the transcripts present in a cell, including messenger RNA (mRNA) as well as all non-coding RNAs. By looking at the whole transcriptome, researchers are able to map exons and introns and to identify splicing variants. Notably, most whole transcription library preparation protocols include a step to remove ribosomal RNA (rRNA), which would otherwise take up the majority of the sequencing reads. Depletion of rRNA is commonly accomplished using a kit, for example, Ribo-Zero Plus rRNA Depletion Kit from IIlumina and Seq RiboFree Total RNA Library Kit from Zymo. In other cases, a more targeted RNA-Seq protocol is used to look at a specific type of RNA. For example, mRNA-seq is commonly used to selectively study the “coding” part of the genome, which accounts for only 1-2% of the entire transcriptome. Enriching a sample for mRNA increases the sequencing depth achieved for coding genes, enabling identification of rare transcripts and variants. Polyadenylated mRNAs are commonly enriched for using oligo dT beads (for example, Dynabeads™ from Invitrogen). This enrichment step can be performed either on isolated total RNA or on crude cellular lysate. Targeted approaches have also been developed for the analysis of microRNAs (miRNAs) and small interfering RNAs (siRNAs). These RNAs are commonly isolated using kits that have been designed to efficiently recover small RNAs (for example, mirVana™ miRNA Isolation Kit from Invitrogen).

After RNA has been extracted from the sample, the next major step is to convert the RNA into a form that is suitable for next-generation sequencing (NGS). Through a series of steps, the RNA is converted into a collection of DNA fragments known as a “sequencing library.” After the library has been sequenced, the resulting sequencing “reads” are aligned to a reference genome or transcriptome to determine the expression profile of the analyzed cells. In some cases, library preparation is automated to enable higher sample throughput, minimize errors, and reduce hands-on time. Fully automated library preparation can be performed, for example, using a liquid handling robot (for example, SciClone® NGSx from PerkinElmer).

For sequencing, RNA is converted to more stable, double-stranded complementary DNA (cDNA) using reverse transcription (RT). In some cases reverse transcription is performed directly on a sample lysate, prior to RNA isolation. In other cases, reverse transcription is performed on isolated RNA. Reverse transcription is catalyzed by reverse transcriptase, an enzyme that uses an RNA template and a short primer complementary to the 3′ end of the RNA to synthesize a complementary strand of cDNA. This first strand of cDNA is then made double-stranded, either by subjecting it to PCR or using a combination of DNA Polymerase I and DNA Ligase. In the latter method, an RNase (for example, RNase H) is commonly used to digest the RNA strand, allowing the first cDNA strand to serve as a template for synthesis of the second cDNA strand. Many reverse transcriptases are commercially available, including Avian Myeloblastosis Virus (AMV) reverse transcriptases (for example, AMV Reverse Transcriptase from New England BioLabs) and Moloney Murine Leukemia Virus (M-MuLV, MMLV) reverse transcriptases (for example, SMARTscribe™ from Clontech, SuperScript II™ from Life Technologies, and Maxima H Minus™ from Thermo Scientific). Notably, many of the available reverse transcriptases have been engineered for improved thermostability or efficiency (for example, by eliminating 3′→5′ exonuclease activity or reducing RNase H activity).

The primers, which serve as a starting point for synthesis of the new strand, may be random primers (for example, for RT of any RNA), oligo dT primers (for example, for RT of mRNA), or gene-specific primers (for example, for RT of specific target RNAs). Following reverse transcription, an exonuclease (for example, Exonuclease I) may be added to the samples to degrade any primers that remain from the reaction, preventing them from interfering in subsequent amplification steps.

Because most sequencing technologies cannot readily analyze long DNA strands, DNA is commonly fragmented into uniform pieces prior to sequencing. The optimal fragment length depends on both the sample type and the sequencing platform to be used. For example, whole genome sequencing typically works best with fragments of DNA that are ˜350 bp long, while targeted sequencing using hybridization capture (see Section 2G) works best with fragments of DNA that are ˜200 bp long. In some cases, fragmentation is performed after reverse transcription (for example, on cDNA). Suitable methods for fragmenting DNA include physical methods (for example, using sonication, acoustics, nebulization, centrifugal force, needles, or hydrodynamics), enzymatic methods (for example, using NEBNext dsDNA Fragmentase from New England BioLabs), and tagmentation (for example, using the Nextera™ system from Illumina). In other cases, fragmentation is performed prior to reverse transcription (for example, on RNA). In addition to the fragmentation methods that are suitable to DNA, RNA may also be fragmented using heat and magnesium (for example, using the KAPA Hyper Prep Kit from Roche).

A size selection step may subsequently be performed to enrich the library for fragments of an optimal length or range of lengths. Traditionally, size selection was accomplished by separating differentially sized fragments using agarose gel electrophoresis, cutting out the fragments of the desired sizes, and performing a gel extraction (for example, using a MinElute Gel Extraction Kit™ from Qiagen). However, size selection is now commonly accomplished using magnetic bead-based systems (for example, AMPure XP™ from Beckman Coulter, ProNex® Size-Selective Purification System from Promega).

Prior to sequencing, the cDNA fragments are ligated to sequencing adapters. Sequencing adapters are short DNA oligonucleotides that contain (1) sequences needed to amplify the cDNA fragment during the sequencing reaction, and (2) sequences that interact with the NGS platform (for example, the surface of the Illumina flow-cell or Ion Torrent beads). Accordingly, adapters must be selected based on the sequencing platform that is to be used.

Libraries from multiple samples are commonly pooled and analyzed in a single sequencing run (see Section 2F). To track the source of each cDNA in a pooled sample, a unique molecular barcode (or combination of multiple barcodes) is included in the adapters that are ligated to the cDNA fragments in each library. During the sequencing reaction, the sequencer reads this barcode sequence in addition to the cDNA's biological base sequence. The barcodes are then used to assign each cDNA to its sample of origin during data analysis, a process termed “demultiplexing”. The indexing strategy used for a sequencing reaction should be selected based on the number of pooled samples and the level of accuracy desired. For example, unique dual indexing, in which unique identifiers are added to both ends of the cDNA fragments, is commonly used to ensure that libraries will demultiplex with high accuracy. Adapters may also include unique molecular identifiers (UMIs), short sequences, often with degenerate bases, that incorporate a unique barcode onto each molecule within a given sample library. UMIs reduce the rate of false-positive variant calls and increase sensitivity of variant detection by allowing true variants to be distinguished from errors introduced during library preparation, target enrichment, or sequencing. Many index sequences and adapter sets are commercially available including, for example, SeqCap Dual End Adapters from Roche, xGen Dual Index UMI Adapters from IDT, and TruSeq UD Indexes from Illumina.

Amplification. While it may not be required for some sequencing applications, library preparation typically includes at least one amplification step to enrich for sequencing-competent DNA fragments (for example, fragments with adapter ligated ends) and to generate a sufficient amount of library material for downstream processing. Amplification may be performed using a standard polymerase chain reaction (PCR) technique. However, when possible, care should be taken to minimize amplification bias and limit the introduction of sequencing artifacts. This is accomplished through selection of an appropriate enzyme and protocol parameters. To this end, several companies offer high-fidelity DNA polymerases (for example, KAPA HiFi DNA Polymerase from Roche), which have been shown to produce more accurate sequencing data. Often these DNA polymerases are purchased as part of a PCR master mix (for example, NEBNext® High-Fidelity 2X PCR Master Mix from New England BioLabs) or as part of a kit (for example, KAPA HiFi Library Amplification kit by Roche). Those of skill in the art will appreciate that PCR conditions must be fine-tuned for each sequencing experiment, even when a highly-optimized PCR protocol is used. For example, depending on the initial concentration of DNA in the library and on the input requirement of the sequencer to be used, it may be desirable to subject the library to anywhere from 4-14 cycles of PCR. In some cases, library preparation protocols include multiple rounds of library amplification. For example, in some cases, an additional round of amplification followed by PCR clean-up is performed after the libraries have been pooled.

Following PCR, the amplified DNA is typically purified to remove enzymes, nucleotides, primers, and buffer components that remain from the reaction. Purification is commonly accomplished using phenol-chloroform extraction followed by ethanol precipitation or using a spin column that contains a silica matrix to which DNA selectively binds in the presence of chaotropic salts. Many column-based PCR cleanup kits are commercially available including, for example, those from Qiagen (for example, MinElute PCR Purification Kit), Zymo Research™ (DNA Clean & Concentrator™-5), and Invitrogen (for example, PureLink™ PCR Purification Kit). Alternatively, purification may be accomplished using paramagnetic beads (for example, Axygen™ AxyPrep Mag™ PCR Clean-up Kit).

To keep sequencing cost-effective, researchers often pool together multiple libraries, each with a unique barcode (see section 2C), to be sequenced in a single run. The sequencer to be used and the desired sequencing depth should dictate the number of samples that are pooled. For example, for some applications it is advantageous to pool fewer than 12 libraries to achieve greater sequencing depth, whereas for other applications it may be advisable to pool more than 100 libraries. Importantly, if multiple libraries are sequenced in a single run, care should be taken to ensure that the sequencing coverage is roughly equal for each library. To this end, an equal amount of each library (based on molarity) should be pooled. Further, the total molarity of the pooled libraries must be compatible with the sequencer. Thus, it is important to accurately quantify the DNA in the libraries (for example, using the methods discussed in herein) and to perform the necessary calculations before pooling the libraries. In some cases, to achieve a suitable total molarity, it may be necessary to concentrate the pooled libraries, for example, using a vacufuge.

For some applications, it is not necessary to sequence the entire transcriptome of a sample. Instead, “targeted sequencing” may be used to study a select set of genes or specific genomic elements. Libraries that are enriched for target sequences are commonly prepared using hybridization based methods (for example, hybridization capture-based target enrichment). Hybridization may be performed either on a solid surface (microarray) or in solution. In the solution based method, a pool of biotinylated oligonucleotide probes that specifically hybridize with the genes or genomic elements of interest is added to the library. The probes are then captured and purified using streptavidin-coated magnetic beads, and the sequences that hybridized to these probes are subsequently amplified and sequenced. Many probe panels for library enrichment are commercially available, including those from IDT (for example, xGen Exome Research Panel v1.0 probes) and Roche (for example, SeqCap® probes). Importantly, many available probe panels can be customized, allowing investigators to design sets of capture probes that are precisely tailored to a particular application. In addition, many kits (for example, SeqCap EZ MedExome Target Enrichment Kit from Roche) and hybridization mixes (for example, xGen Lockdown from IDT) that facilitate target enrichment are available for purchase. In some cases, it may be advantageous to treat the libraries with reagents that reduce off-target capture prior to performing target enrichment. For example, libraries are commonly treated with oligonucleotides that bind to adapter sequences (for example, xGen Blocking Oligos) or to repetitive sequences (for example, human Cot DNA) to reduce non-specific binding to the capture probes.

In one example, a sequencer may generate a BCL file. A BCL file may include raw image data of a plurality of patient specimens which are sequenced. BCL image data is an image of the flow cell across each cycle during sequencing. A cycle may be implemented by illuminating a patient specimen with a specific wavelength of electromagnetic radiation, generating a plurality of images which may be processed into base calls via BCL to FASTQ processing algorithms which identify which base pairs are present at each cycle. The resulting FASTQ may then comprise the entirety of reads for each patient specimen paired with a quality metric in a range from 0 to 64 where a 64 is the best quality and a 0 is the worst quality. A patient's tumor specimen and a patient's normal specimen may be matched after sequencing such that a tumor-normal analysis may be performed.

Each FASTQ file contains reads that may be paired-end or single reads, and may be short-reads or long-reads, where each read represents one detected sequence of nucleotides in a DNA molecule that was isolated from the patient sample, a copy of the DNA molecule or a cDNA molecule or copy of the cDNA molecule, where the cDNA molecule was derived from an RNA molecule isolated from the patient sample, detected by the sequencer. Each read in the FASTQ file is also associated with a quality rating. The quality rating may reflect the likelihood that an error occurred during the sequencing procedure that affected the associated read.

One use of RNA-seq data is to identify genes that are differentially expressed between two or more experimental groups. For example, RNA sequencing data can be used to identify genes that are expressed at significantly higher or lower levels in patients (for example, patients having cancer, autoimmune disease(s), an infection, and/or transplantation requirement) as compared to healthy individuals. This may be accomplished by performing a statistical analysis to compare the normalized read count of each gene across the different experimental groups. The aim of this analysis is to determine whether any observed difference in read count is significant, i.e., whether it is greater than what would be expected just due to natural random variation.

Several data processing steps may be performed to prepare the raw sequencing data for analysis. Sequencing data is typically supplied in FASTQ format, in which each sequencing read is associated with a quality score. First, the data is processed to remove sequencing artifacts, e.g., adaptor sequences and low-complexity reads. Sequencing errors are identified based on the read quality score and are removed or corrected. Publicly available tools, such as TagDust, SeqTrim, and Quake, can be used to perform these “data grooming” steps.

During the next stage of data processing, the reads are aligned to a reference genome using an alignment tool. Several publicly available tools can be used for this step including, for example, Kallisto or other pseudo alignment tools, or alignment tools including TopHat, Cufflinks, and Scripture. These programs can be used to reconstruct transcripts, identify variants, and quantitate expression levels for each transcript and gene.

After the reads have been aligned and quantitated, a differential expression analysis may be performed. Statistical methods that are commonly used for differential expression analysis include those based on negative binomial distributions (e.g., edgeR and DESeq) and Bayesian approaches based on a negative binomial model (e.g., baySeq and EBSeq).

In certain aspects, the bioinformatics pipeline includes the systems and methods disclosed in this document. The bioinformatics methods may include filtering NGS reads (for example, according to quality scores or other characteristics associated with each read), aligning reads to a reference genome, detecting fusions having a 5′ partner sequence and a 3′ partner sequence, analyzing read depths or other potentially relevant coverage factors and the status of a partner sequence as in-frame or out-of-frame, labeling fusions, and storing the labeled fusion data in a database. In one example, in-frame means that the number of nucleotides between the final nucleotide of the 3′ partner sequence and the starting nucleotide of the 5′ partner sequence is a number divisible by three. If that condition is not met, the fusion sequence is classified as out-of-frame.

In some embodiments, the bioinformatics pipeline may filter FASTQ data from the corresponding sequence data file for each respective biological sample. Such filtering may include correcting or masking sequencer errors and removing (trimming) low quality sequences or bases, adapter sequences, contaminations, chimeric reads, overrepresented sequences, biases caused by library preparation, amplification, or capture, and other errors.

The workflow 200 illustrates steps for obtaining a biological sample, extracting nucleic acids from the biological sample, and sequencing the isolated nucleic acids, in some embodiments, sequencing data used in the improved systems and methods described herein (for example, which include improved methods for fusion pathogenicity scoring) is obtained by receiving previously generated sequence reads, in electronic form.

Referring again to FIG. 2A, nucleic acid sequencing data 122 generated from the one or more patient samples is then evaluated (for example, via variant analysis 208) in a bioinformatics pipeline, for example, using bioinformatics module 140 of system 100, to identify genomic alterations and other metrics in the cancer genome of the patient. An example overview for a bioinformatics pipeline is described below. Advantageously, in some embodiments, the present disclosure improves bioinformatics pipelines, like pipeline 206, by improving fusion pathogenicity.

FIG. 4A illustrates an example bioinformatics pipeline 206 (for example, as used for feature extraction in the workflows illustrated in FIGS. 2A and 3) for providing clinical support for precision oncology. As shown in FIG. 4A, sequencing data 122 obtained from the wet lab processing 204 (for example, sequence reads 314) is input into the pipeline.

In some embodiments, the sequencing data is processed (for example, using sequence data processing module 141) to prepare it for genomic feature identification 385. For instance, in some embodiments as described above, the sequencing data is present in a native file format provided by the sequencer. Accordingly, in some embodiments, the system (for example, system 100) applies a pre-processing algorithm 142 to convert the file format (318) to one that is recognized by one or more upstream processing algorithms. For example, BCL file outputs from a sequencer can be converted to a FASTQ file format using the bcl2fastq or bcl2fastq2 conversion software (Illumina®). FASTQ format is a text-based format for storing both a biological sequence, such as nucleotide sequence, and its corresponding quality scores. These FASTQ files are analyzed to determine what genetic variants, copy number changes, etc., are present in the sample.

In some embodiments, other preprocessing steps are performed, for example, filtering sequence reads 122 based on a desired quality, for example, size and/or quality of the base calling. In some embodiments, quality control checks are performed to ensure the data is sufficient for variant calling. For instance, entire reads, individual nucleotides, or multiple nucleotides that are likely to have errors may be discarded based on the quality rating associated with the read in the FASTQ file, the known error rate of the sequencer, and/or a comparison between each nucleotide in the read and one or more nucleotides in other reads that has been aligned to the same location in the reference genome. Filtering may be done in part or in its entirety by various software tools, for example, a software tool such as Skewer. See, Jiang, H. et al., BMC Bioinformatics 15(182):1-12 (2014). FASTQ files may be analyzed for rapid assessment of quality control and reads, for example, by a sequencing data QC software such as AfterQC, Kraken, RNA-SeQC, FastQC, or another similar software program. For paired-end reads, reads may be merged.

In some embodiments, when both a liquid biopsy sample and a normal tissue sample from the patient are sequenced, two FASTQ output files are generated, one for the liquid biopsy sample and one for the normal tissue sample. A ‘matched’ (for example, panel-specific) workflow is run to jointly analyze the liquid biopsy-normal matched FASTQ files. When a matched normal sample is not available from the patient, FASTQ files from the liquid biopsy sample are analyzed in the ‘tumor-only’ mode. See, for example, FIG. 4B, which shows an exemplary isolate analysis pipeline 209. If two or more patient samples are processed simultaneously on the same sequencer flow cell, for example, a liquid biopsy sample and a normal tissue sample, a difference in the sequence of the adapters used for each patient sample barcodes nucleic acids extracted from both samples, to associating each read with the correct patient sample and facilitate assignment to the correct FASTQ file.

For efficiency, in some embodiments, the results of paired-end sequencing of each isolate are contained in a split pair of FASTQ files. Forward (Read 1) and reverse (Read 2) sequences of each tumor and normal isolate are stored separately but in the same order and under the same identifier. In some embodiments, the bioinformatics pipeline may filter FASTQ data from each isolate. Such filtering may include correcting or masking sequencer errors and removing (trimming) low quality sequences or bases, adapter sequences, contaminations, chimeric reads, overrepresented sequences, biases caused by library preparation, amplification, or capture, and other errors.

Similarly, in some embodiments, sequencing (312) is performed on a pool of nucleic acid sequencing libraries prepared from different biological samples, for example, from the same or different patients. Accordingly, in some embodiments, the system demultiplexes (320) the data (for example, using demultiplexing algorithm 144) to separate sequence reads into separate files for each sequencing library included in the sequencing pool, for example, based on UMI or patient identifier sequences added to the nucleic acid fragments during sequencing library preparation, as described above. In some embodiments, the demultiplexing algorithm is part of the same software package as one or more pre-processing algorithms 142. For instance, the bcl2fastq or bcl2fastq2 conversion software (Illumina®) include instructions for both converting the native file format output from the sequencer and demultiplexing sequence reads 122 output from the reaction.

The sequence reads are then aligned (322), for example, using an alignment algorithm 143, to a reference sequence construct 158, for example, a reference genome, reference exome, or other reference construct prepared for a particular targeted-panel sequencing reaction. For example, in some embodiments, individual sequence reads 123, in electronic form (for example, in FASTQ files), are aligned against a reference sequence construct for the species of the subject (for example, a reference human genome) by identifying a sequence in a region of the reference sequence construct that best matches the sequence of nucleotides in the sequence read. In some embodiments, the sequence reads are aligned to a reference exome or reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. Any of a variety of alignment tools can be used for this task.

For instance, local sequence alignment algorithms compare subsequences of different lengths in the query sequence (for example, sequence read) to subsequences in the subject sequence (for example, reference construct) to create the best alignment for each portion of the query sequence. In contrast, global sequence alignment algorithms align the entirety of the sequences, for example, end to end. Examples of local sequence alignment algorithms include the Smith-Waterman algorithm (see, for example, Smith and Waterman, J Mol. Biol., 147(1):195-97 (1981), which is incorporated herein by reference), Lalign (see, for example, Huang and Miller, Adv. Appl. Math, 12:337-57 (1991), which is incorporated by reference herein), and PatternHunter (see, for example, Ma B. et al., Bioinformatics, 18(3):440-45 (2002), which is incorporated by reference herein).

In some embodiments, the read mapping process starts by building an index of either the reference genome or the reads, which is then used to retrieve the set of positions in the reference sequence where the reads are more likely to align. Once this subset of possible mapping locations has been identified, alignment is performed in these candidate regions with slower and more sensitive algorithms. See, for example, Hatem et al., 2013, “Benchmarking short sequence mapping tools,” BMC Bioinformatics 14: p. 184; and Flicek and Birney, 2009, “Sense from sequence reads: methods for alignment and assembly,” Nat Methods 6(Suppl. 11), S6-S12, each of which is hereby incorporated by reference. In some embodiments, the mapping tools methodology makes use of a hash table or a Burrows-Wheeler transform (BWT). See, for example, Li and Homer, 2010, “A survey of sequence alignment algorithms for next-generation sequencing,” Brief Bioinformatics 11, pp. 473-483, which is hereby incorporated by reference.

Other software programs designed to align reads include, for example, Novoalign (Novocraft, Inc.), Bowtie, Burrows Wheeler Aligner (BWA), and/or programs that use a Smith-Waterman algorithm. Candidate reference genomes include, for example, hg19, GRCh38, hg38, GRCh37, and/or other reference genomes developed by the Genome Reference Consortium. In some embodiments, the alignment generates a SAM file, which stores the locations of the start and end of each read according to coordinates in the reference genome and the coverage (number of reads) for each nucleotide in the reference genome.

For example, in some embodiments, each read of a FASTQ file is aligned to a location in the human genome having a sequence that best matches the sequence of nucleotides in the read. There are many software programs designed to align reads, for example, Novoalign (Novocraft, Inc.), Bowtie, Burrows Wheeler Aligner (BWA), programs that use a Smith-Waterman algorithm, etc. Alignment may be directed using a reference genome (for example, hg19, GRCh38, hg38, GRCh37, other reference genomes developed by the Genome Reference Consortium, etc.) by comparing the nucleotide sequences in each read with portions of the nucleotide sequence in the reference genome to determine the portion of the reference genome sequence that is most likely to correspond to the sequence in the read. In some embodiments, one or more SAM files are generated for the alignment, which store the locations of the start and end of each read according to coordinates in the reference genome and the coverage (number of reads) for each nucleotide in the reference genome. The SAM files may be converted to BAM files. In some embodiments, the BAM files are sorted and duplicate reads are marked for deletion, resulting in de-duplicated BAM files.

This process produces a tumor BAM file, and a normal BAM file (when available). In some embodiments, where both a liquid biopsy sample and a normal tissue sample are analyzed, this process produces a liquid biopsy BAM file (for example, Liquid BAM 124-1-i-cf) and a normal BAM file (for example, Germline BAM 124-1-i-g), as illustrated in FIG. 4A. In some embodiments, BAM files may be analyzed to detect genetic variants and other genetic features, including single nucleotide variants (SNVs), copy number variants (CNVs), gene rearrangements, etc.

In some embodiments, the sequencing data is normalized, for example, to account for pull-down, amplification, and/or sequencing bias (for example, mappability, GC bias etc.). See, for example, Schwartz et al., PLoS ONE 6(1):e16685 (2011) and Benjamini and Speed, Nucleic Acids Research 40(10):e72 (2012), the contents of which are hereby incorporated by reference, in their entireties, for all purposes.

In some embodiments, SAM files generated after alignment are converted to BAM files 124. Thus, after preprocessing sequencing data generated for a pooled sequencing reaction, BAM files are generated for each of the sequencing libraries present in the master sequencing pools. For example, as illustrated in FIG. 4A, separate BAM files are generated for each of three samples acquired from subject 1 at time i (for example, tumor 124-1-i-t corresponding to alignments of sequence reads of nucleic acids isolated from a solid tumor sample from subject 1, Liquid 124-1-i-cf corresponding to alignments of sequence reads of nucleic acids isolated from a liquid biopsy sample from subject 1, and Germline 124-1-i-g corresponding to alignments of sequence reads of nucleic acids isolated from a normal tissue sample from subject 1), and one or more samples acquired from one or more additional subjects at time j (for example, Tumor BAM 124-2-j-t corresponding to alignments of sequence reads of nucleic acids isolated from a solid tumor sample from subject 2). In some embodiments, BAM files are sorted, and duplicate reads are marked for deletion, resulting in de-duplicated BAM files. For example, tools like Sam BAMBA mark and filter duplicate alignments in the sorted BAM files.

Many of the embodiments described below, in conjunction with FIG. 4A, relate to analyses performed using sequencing data from cfDNA of a cancer patient, for example, obtained from a liquid biopsy sample of the patient. Generally, these embodiments are independent and, thus, not reliant upon any particular sequencing data generation methods, for example, sample preparation, sequencing, and/or data pre-processing methodologies. However, in some embodiments, the methods described below include one or more steps of generating sequencing data, as illustrated in FIGS. 2A and 3.

Alignment files prepared as described above (for example, BAM files 124) are then passed to a feature extraction module 145, where the sequences are analyzed (324) to identify genomic alterations (for example, SNVs/MNVs, indels, genomic rearrangements, copy number variations, etc.) and/or determine various characteristics of the patient's cancer (for example, MSI status, TMB, tumor ploidy, HRD status, tumor fraction, tumor purity, methylation patterns, etc.). Many software packages for identifying genomic alterations are known in the art, for example, freebayes, PolyBayse, samtools, GATK, pindel, SAMtools, Breakdancer, Cortex, Crest, Delly, Gridss, Hydra, Lumpy, Manta, and Socrates. For a review of many of these variant calling packages see, for example, Cameron, D.L. et al., Nat. Commun., 10(3240):1-11 (2019), the content of which is hereby incorporated by reference, in its entirety, for all purposes. Generally, these software packages identify variants in sorted SAM or BAM files 124, relative to one or more reference sequence constructs 158. The software packages then output a file for example, a raw VCF (variant call format), listing the variants (for example, genomic features 131) called and identifying their location relevant to the reference sequence construct (for example, where the sequence of the sample nucleic acids differ from the corresponding sequence in the reference construct). In some embodiments, system 100 digests the contents of the native output file to populate feature data 125 in test patient data store 120. In other embodiments, the native output file serves as the record of these genomic features 131 in test patient data store 120.

Generally, the systems described herein can employ any combination of available variant calling software packages and internally developed variant identification algorithm. In some embodiments, the output of a particular algorithm of a variant calling software is further evaluated, for example, to improve variant identification. Accordingly, in some embodiments, system 100 employs an available variant calling software package to perform some of all of the functionality of one or more of the algorithms shown in feature extraction module 145.

In some embodiments, as illustrated in FIG. 1A, separate algorithms (or the same algorithm implemented using different parameters) are applied to identify variants unique to the cancer genome of the patient and variants existing in the germline of the subject. In other embodiments, variants are identified indiscriminately and later classified as either germ line or somatic, for example, based on sequencing data, population data, or a combination thereof. In some embodiments, variants are classified as germline variants, and/or non-actionable variants, when they are represented in the population above a threshold level, for example, as determined using a population database such as ExAC or gnomAD. For instance, in some embodiments, variants that are represented in at least 1% of the alleles in a population are annotated as germline and/or non-actionable. In other embodiments, variants that are represented in at least 2%, at least 3%, at least 4%, at least 5%, at least 7.5%, at least 10%, or more of the alleles in a population are annotated as germline and/or non-actionable. In some embodiments, sequencing data from a matched sample from the patient, for example, a normal tissue sample, is used to annotate variants identified in a cancerous sample from the subject. That is, variants that are present in both the cancerous sample and the normal sample represent those variants that were in the germ line prior to the patient developing cancer, and can be annotated as germline variants.

In various aspects, the detected genetic variants and genetic features are analyzed as a form of quality control. For example, a pattern of detected genetic variants or features may indicate an issue related to the sample, sequencing procedure, and/or bioinformatics pipeline (for example, example, contamination of the sample, mislabeling of the sample, a change in reagents, a change in the sequencing procedure and/or bioinformatics pipeline, etc.).

This particular workflow is only an example of one possible collection and arrangement of algorithms for feature extraction from sequencing data 124. Generally, any combination of the modules and algorithms of feature extraction module 145, for example, illustrated in FIG. 1A, can be used for a bioinformatics pipeline. For instance, in some embodiments, an architecture useful for the methods and systems described herein includes at least one of the modules or variant calling algorithms shown in feature extraction module 145. In some embodiments, an architecture includes at least 2, 3, 4, 5, 6, 7, 8, 9, 10, or more of the modules or variant calling algorithms shown in feature extraction module 145. Further, in some embodiments, feature extraction modules and/or algorithms not illustrated in FIG. 1A find use in the methods and systems described herein.

In some embodiments, variant analysis of aligned sequence reads, for example, in SAM or BAM format, includes identification of single nucleotide variants (SNVs), multiple nucleotide variants (MNVs), indels (for example, nucleotide additions and deletions), and/or genomic rearrangements (for example, inversions, translocations, and gene fusions) using variant identification module 146, for example, which includes a SNV/MNV calling algorithm (for example, SNV/MNV calling algorithm 147), an indel calling algorithm (for example, indel calling algorithm 148), and/or one or more genomic rearrangement calling algorithms (for example, genomic rearrangement calling algorithm 149). Essentially, the module first identifies a difference between the sequence of an aligned sequence read 124 and the reference sequence to which the sequence read is aligned (for example, an SNV/MNV, an indel, or a genomic rearrangement) and makes a record of the variant, for example, in a variant call format (VCF) file. For instance, software packages such as freebayes and pindel are used to call variants using sorted BAM files and reference BED files as the input. For a review of variant calling packages see, for example, Cameron, D.L. et al., Nat. Commun., 10(3240):1-11 (2019). A raw VCF file (variant call format) file is output, showing the locations where the nucleotide base in the sample is not the same as the nucleotide base in that position in the reference sequence construct.

In some embodiments, raw VCF data is then normalized, for example, by parsimony and left alignment. For example, software packages such as vcfbreakmulti and vt are used to normalize multi-nucleotide polymorphic variants in the raw VCF file and a variant normalized VCF file is output. See, for example, E. Garrison, “Vcflib: A C++ library for parsing and manipulating VCF files, GitHub https://github.com/ekg/vcflib (2012), the content of which is hereby incorporated by reference, in its entirety, for all purposes. In some embodiments, a normalization algorithm is included within the architecture of a broader variant identification software package.

An algorithm is then used to annotate the variants in the (for example, normalized) VCF file, for example, determines the source of the variation, for example, whether the variant is from the germ line of the subject (for example, a germ line variant), a cancerous tissue (for example, a somatic variant), a sequencing error, or of an undeterminable source. In some embodiments, an annotation algorithm is included within the architecture of a broader variant identification software package. However, in some embodiments, an external annotation algorithm is applied to (for example, normalized) VCF data obtained from a conventional variant identification software package. The choice to use a particular annotation algorithm is well within the purview of the skilled artisan, and in some embodiments is based upon the data being annotated.

For example, in some embodiments, where both a liquid biopsy sample and a normal tissue sample of the patient are analyzed, variants identified in the normal tissue sample inform annotation of the variants in the liquid biopsy sample. In some embodiments, where a particular variant is identified in the normal tissue sample, that variant is annotated as a germ line variant in the liquid biopsy sample. Similarly, in some embodiments, where a particular variant identified in the liquid biopsy sample is not identified in the normal tissue sample, the variant is annotated as a somatic variant when the variant otherwise satisfies any additional criteria placed on somatic variant calling, for example, a threshold variant allele frequency (VAF) in the sample.

By contrast, in some embodiments, where only a liquid biopsy sample is being analyzed, the annotation algorithm relies on other characteristics of the variant in order to annotate the origin of the variant. For instance, in some embodiments, the annotation algorithm evaluates the VAF of the variant in the sample, for example, alone or in combination with additional characteristics of the sample, for example, tumor fraction. Accordingly, in some embodiments, where the VAF is within a first range encompassing a value that corresponds to a 1:1 distribution of variant and reference alleles in the sample, the algorithm annotates the variant as a germline variant, because it is presumably represented in cfDNA originating from both normal and cancer tissues. Similarly, in some embodiments, where the VAF is below a baseline variant threshold, the algorithm annotates the variant as undeterminable, because there is not sufficient evidence to distinguish between the possibility that the variant arose as a result of an amplification or sequencing error and the possibility that the variant originated from a cancerous tissue. Similarly, in some embodiments, where the VAF falls between the first range and the baseline variant threshold, the algorithm annotates the variant as a somatic variant.

In some embodiments, the baseline variant threshold is a value from 0.01% VAF to 0.5% VAF. In some embodiments, the baseline variant threshold is a value from 0.05% VAF to 0.35% VAF. In some embodiments, the baseline variant threshold is a value from 0.1% VAF to 0.25% VAF. In some embodiments, the baseline variant threshold is about 0.01% VAF, 0.015% VAF, 0.02% VAF, 0.025% VAF, 0.03% VAF, 0.035% VAF, 0.04% VAF, 0.045% VAF, 0.05% VAF, 0.06% VAF, 0.07% VAF, 0.075% VAF, 0.08% VAF, 0.09% VAF, 0.1% VAF, 0.15% VAF, 0.2% VAF, 0.25% VAF, 0.3% VAF, 0.35% VAF, 0.4% VAF, 0.45% VAF, 0.5% VAF, or greater. In some embodiments, the baseline variant threshold is different for variants located in a first region, for example, a region identified as a mutational hotspot and/or having high genomic complexity, than for variants located in a second region, for example, a region that is not identified as a mutational hotspot and/or having average genomic complexity. For example, in some embodiments, the baseline variant threshold is a value from 0.01% to 0.25% for variants located in the first region and is a value from 0.1% to 0.5% for variants located in the second region. In some embodiments, a baseline variant threshold is influenced by the sequencing depth of the reaction, for example, a locus-specific sequencing depth and/or an average sequencing depth (for example, across a targeted panel and/or complete reference sequence construct). In some embodiments, the baseline variant threshold is dependent upon the type of variant being detected. For example, in some embodiments, different baseline variant thresholds are set for SNPs/MNVs than for indels and/or genomic rearrangements. For instance, while an apparent SNP may be introduced by amplification and/or sequencing errors, it is much less likely that a genomic rearrangement is introduced this way and, thus, a lower baseline variant threshold may be appropriate for genomic rearrangements than for SNPs/MNVs.

In some embodiments, one or more additional criteria are required to be satisfied before a variant can be annotated as a somatic variant. For instance, in some embodiments, a threshold number of unique sequence reads encompassing the variant must be present to annotate the variant as somatic. In some embodiments, the threshold number of unique sequence reads is only applied when certain conditions are met, for example, when the variant allele is located in a region of average genomic complexity. In some embodiments, a threshold sequencing coverage, for example, a locus-specific and/or an average sequencing depth (for example, across a targeted panel and/or complete reference sequence construct) must be satisfied to annotate the variant as somatic. In some embodiments, bases contributing to the variant must satisfy a threshold mapping quality to annotate the variant as somatic. In some embodiments, alignments contributing to the variant must satisfy a threshold alignment quality to annotate the variant as somatic. In some embodiments, one or more genomic regions is blacklisted, preventing somatic variant annotation for variants falling within the region. In some embodiments, any combination of the additional criteria, as well as additional criteria not listed above, may be applied to the variant calling process. Again, in some embodiments, different criteria are applied to the annotation of different types of variants.

In some embodiments, genomic rearrangements (for example, inversions, translocations, and gene fusions) are detected following de-multiplexing by aligning tumor FASTQ files against a human reference genome using a local alignment algorithm, such as BWA. In some embodiments, DNA reads are sorted and duplicates may be marked with a software, for example, SAMBlaster. Discordant and split reads may be further identified and separated. These data may be read into a software, for example, LUMPY, for structural variant detection, including candidate fusion detection, as part of a fusion detection pipeline. Examples of fusion detection software packages include Pizzly, STAR, MOJO, etc. (see https://github.com/pmelsted/pizzly, https://github.com/STAR-Fusion/STAR-Fusion/wiki, https://github.com/cband/MOJO). In some embodiments, the fusion detection pipeline may be hosted on one or more docker images. In some embodiments, structural alterations are grouped by type, recurrence, and presence and stored within a database and displayed through a fusion viewer software tool. The fusion viewer software tool may reference a database, for example, Ensembl, to determine the gene and proximal exons surrounding the breakpoint for any possible transcript generated across the breakpoint. The fusion viewer tool may then place the breakpoint 5′ or 3′ to the subsequent exon in the direction of transcription. For inversions, this orientation may be reversed for the inverted gene. After positioning of the breakpoint, the translated amino acid sequences may be generated for both genes in the chimeric protein, and a plot may be generated containing the remaining functional domains for each protein, as returned from a database, for example, Uniprot.

For instance, in an example implementation, gene rearrangements are detected using the SpeedSeq analysis pipeline. Chiang et al., 2015, “SpeedSeq: ultra-fast personal genome analysis and interpretation,” Nat Methods, (12), pg. 966. Briefly, FASTQ files are aligned to hg19 using BWA. Split reads mapped to multiple positions and read pairs mapped to discordant positions are identified and separated, then utilized to detect gene rearrangements by LUMPY. Layer et al., 2014, “I.M. LUMPY: a probabilistic framework for structural variant discovery,” Genome Biol, (15), pg. 84. Fusions can then be filtered according to the number of supporting reads.

In some embodiments, fusion detection includes analyzing aligned reads to detect reads having two portions where the portions align to non-contiguous regions of a reference genome. Fusion detection may further include localizing breakpoints in data based on misalignments, ascribing and quantifying the technical, supporting reads spanning the breakpoint, and estimating where breakpoints are located in the genome. If the number of split reads and discordant reads detected for a given breakpoint exceeds a threshold value, the group of reads associated with each breakpoint are grouped as a fusion candidate.

In some embodiments, thousands of fusion candidates could be detected per sample. The majority of fusion candidates may be artifacts or passenger fusions and/or irrelevant to tumor biology. The identification of driver or pathogenic fusions that are involved in tumor pathogenesis, therefore, require detailed evaluation of key biological features of a fusion candidate as well as appropriate filtering of artifacts or passenger events.

However, many fusion candidates are not well-studied and it is likely that many pathogenic fusions exist that have not yet been demonstrated to be pathogenic or identified as canonical fusions. In some embodiments, the systems and methods disclosed herein detect fusion candidates that are most likely to be pathogenic.

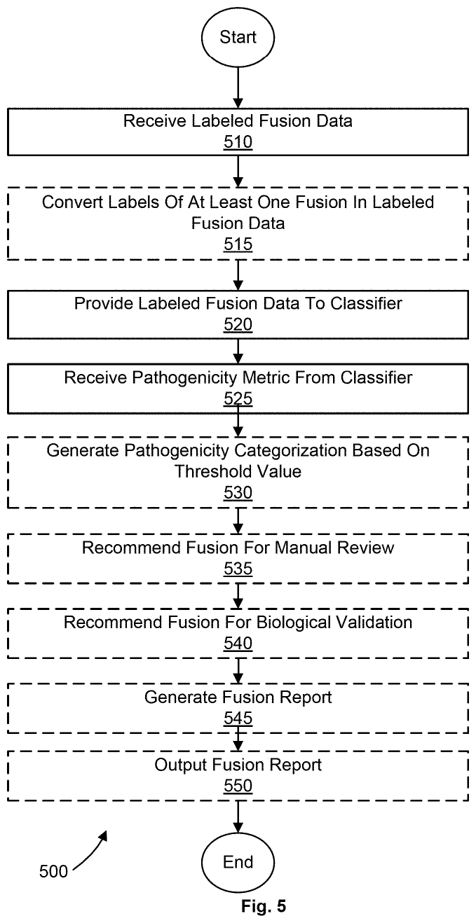

FIG. 5 illustrates an exemplary process 500 to rank fusion events. In some embodiments, the process 500 can rank a fusion event as having a low, medium, or high likelihood of being a pathogenic fusion. The process 500 can be implemented as computer readable instructions on one or more memories or other non-transitory computer readable media, and executed by one or more processors in communication with the one or more memories or other media. In some embodiments, the process 500 can be implemented as computer readable instructions on the persistent memory 112 and/or the non-persistent memory 111 and executed by the processor 102. In some embodiments, the process 500 can be executed by a sequencing system.

At 510, the process 500 can receive labeled fusion data. In some embodiments, the process 500 can query a database of labeled fusion data (for an example of a querying method, see FIG. 8, step 1) or receive a flat file generated by an upstream bioinformatics fusion detection pipeline. The labeled fusion data can include one or more fusions. In some embodiments, each fusion may be associated with a biological specimen and may have a 5′ partner sequence and a 3′ partner sequence that are not contiguous in most specimens but are contiguous in this specimen. Each 5′ partner sequence and each 3′ partner sequence may be either a positive sense (e.g., forward) strand or a negative sense (e.g., reverse) strand.

In some embodiments, the labeled fusion data can include RNA data and/or DNA data. In some embodiments, the labeled fusion data can include RNA-seq and DNA-seq data generated by performing bioinformatics methods on long-read or short-read next generation sequencing (NGS) reads from nucleic acid molecules associated with a biological specimen. The biological specimen may include tissue, blood, saliva, or any specimen collected from a human patient. A tissue sample may be frozen, formalin fixed paraffin embedded (FFPE), fixed to a histopathology slide, or preserved by another method before being processed to generate a pool of nucleic acid molecules to be analyzed by NGS. In some embodiments, the labeled fusion data can include whole transcriptome RNA sequencing data generated based on the specimen. In some embodiments, the specimen can be a tumor.

In some embodiments, each biological specimen can be associated with zero or more fusions, and for each fusion, the 5′ partner sequence and the 3′ partner sequence are not associated with the same underlying genetic sequence in the reference genome. In some embodiments, the 5′ partner sequence and the 3′ partner sequence are not associated with the same gene. In some embodiments, the 5′ partner sequence and the 3′ partner sequence are associated with the same gene, including examples of EGFR fusions due to deletion of an internal EGFR genetic sequence (for example, EGFR vIVa/vIVb). In some embodiments, the labeled fusion data can include one or more detected fusions and/or one or more artifacts.

In some embodiments, for each fusion, the labeled fusion data can include the name of each gene, HGNC gene symbol, and/or Ensembl ID associated with each partner sequence, the chromosomal location coordinates for the start (first) nucleotide and end (last) nucleotide of each partner sequence, and the number of reads that cover both the 5′ partner sequence and the 3′ partner sequence (e.g., reads that span the breakpoint). For an example of labeled fusion data, see FIG. 8B.

In some embodiments, the process 500 can proceed to optional 515. In some embodiments, after 510, the process 500 can proceed to 520. At 515, the process 500 can convert labels of at least one fusion from the labeled fusion data. In some embodiments, the process 500 can proceed to 515 only if the output of a particular bioinformatic pipeline is required to be adapted in order to become input for a particular trained classifier. In some embodiments, the chromosomal location coordinates associated with a fusion are not in accordance with a desired genetic coordinate system, and at 515, the process 500 can transpose the chromosomal location coordinates and convert the chromosomal location coordinates to the desired genetic coordinate system (e.g., hg19 is the desired genetic coordinate system). An example of transposing chromosomal location coordinates is provided further below in conjunction with FIG. 8A. For a given fusion, the process 500 can convert start and end labels of each partner sequence to one of the following labels: genomic start, 5′ breakpoint, 3′ breakpoint, or genomic end, depending on the forward or reverse status of the 5′ sequence partner and the 3′ sequence partner. In some embodiments, the process 500 can adjust the chromosomal locations of the 5′ breakpoint and the 3′ breakpoint, depending on the forward or reverse status of the 5′ sequence partner and the 3′ sequence partner.

If a chromosomal location coordinate is located in an intron (in one example, this is only possible for DNA fusions, not RNA fusions), the process 500 can replace the chromosomal location coordinate with the closest exon coordinate. For a chromosomal location coordinate associated with a 5′ partner sequence, the closest exon coordinate is the nearest upstream edge or boundary of an exon (the exon boundary that is physically closest in the 5′ direction from the intron location coordinate). For a chromosomal location coordinate associated with a 3′ partner sequence, the closest exon coordinate is the nearest downstream edge or boundary of an exon (the exon boundary that is physically closest in the 3′ direction from the intron location coordinate). If multiple isoforms of the nearest exon are available, the process 500 can select the most clinically relevant isoform structure. The most clinically relevant isoform may be the isoform that is most commonly observed for one or more clinical data characteristics (for example, cancer type, specimen origin/tissue type, etc.) associated with the biological specimen. In some embodiments, the most common isoform may be determined by analyzing records of observed isoforms, characterized according to associated clinical data, especially clinical data having characteristics in common with the biological specimen. In some embodiments, the records can be accessed from a database of observed isoforms and associated clinical data.

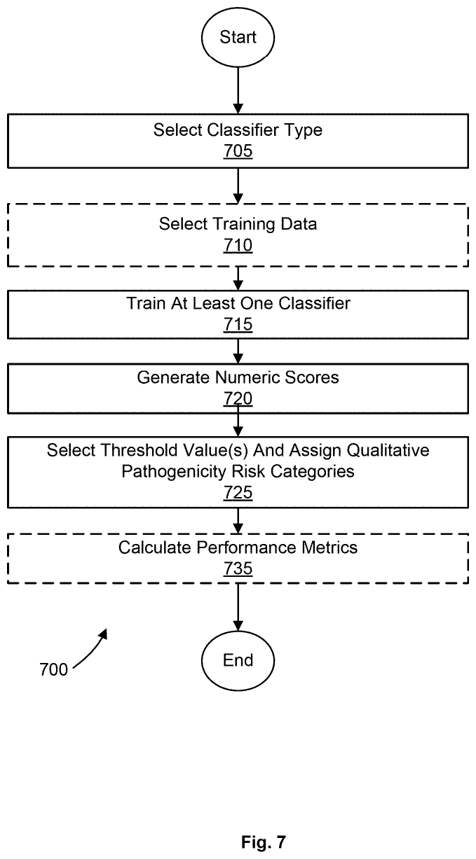

At 520, the process 500 can provide the labeled fusion data to a trained classifier. Specifically, for each fusion included in the fusion data, the process 500 can provide at least a portion of the labeled fusion data to a trained classifier. An exemplary trained classifier 300 will be described further below. The trained classifier may be trained according to the process 700 described below in conjunction with FIG. 7. In some embodiments, for each fusion, the labeled fusion data can include a list of labeled fusion data in a tabular format. In some embodiments, the list can be a .txt file. FIG. 8D provides an example of a list of labeled fusion data. In some embodiments, the labeled data may include a HUGO Gene Nomenclature Committee (HGNC) gene symbol for the 5′ partner sequence, a HUGO Gene Nomenclature Committee (HGNC) gene symbol for the 3′ partner sequence, an Ensembl ID for 5′ partner sequence, an Ensembl ID for 3′ partner sequence, the strandedness of the 5′ partner sequence (either +“forward” or −“reverse), the strandedness of the 3′ partner sequence (either +“forward” or −“reverse), the number or letter denoting the human chromosome on which the 5′ partner sequence is located, the number or letter denoting the human chromosome on which the 3′ partner sequence is located, the genomic coordinate start of 5′ partner sequence, the genomic coordinate end of 5′ partner sequence, the genomic coordinate start of 3′ partner sequence, the genomic coordinate end of 3′ partner sequence, the number of reads spanning the fusion breakpoint, and the total number of high quality reads spanning the fusion breakpoint.

At 525, the process 500 can receive a pathogenicity metric from the trained classifier. The process 500 can generate at least one pathogenicity metric for each fusion. In some embodiments, the pathogenicity metric can be a numeric pathogenicity score for each fusion. In some embodiments, for each fusion, the trained classifier can generate at least one numeric score indicating the likelihood that the fusion is pathogenic. In some embodiments, the pathogenicity metric can be a numeric risk score in the range of 0 to 1. In some embodiments, one of the numeric scores is a DriverScore. In some embodiments, the pathogenicity metric can be a categorization of pathogenicity (e.g., low, medium, and/or high). In these embodiments, the trained classifier can generate a numeric risk score and generate the categorization based on one or more thresholds as described below.

In some embodiments, the process 500 can proceed to optional 530. At 530, the process 500 can generate a pathogenicity categorization based on one or more threshold values. In some embodiments, the process 500 can compare each numeric score generated by the trained classifier to a threshold value in order to assign a pathogenicity risk category to the fusion associated with the numeric score. The process 500 may also compare additional numeric characteristics associated with a fusion to a threshold value in order to select the pathogenicity risk categorization.

In some embodiments, the pathogenicity risk categories can include low, medium, and/or high. For each fusion, the associated numeric score generated by the trained classifier is compared to one threshold value and the associated number of reads (high quality reads or all reads) spanning the breakpoint are compared to a second threshold value. If both the numeric score and the number of spanning reads exceed their respective thresholds, the process 500 can label the associated fusion as having a high likelihood of pathogenicity. If only one of either the numeric score or the number of spanning reads exceeds its respective threshold, the process 500 can label the associated fusion as having a medium likelihood of pathogenicity. If neither the numeric score nor the number of spanning reads exceed their respective thresholds, the process 500 can label the associated fusion as having a low likelihood of pathogenicity.

In some embodiments, there is a low/medium threshold and a medium/high threshold such that the numeric score generated by the trained classifier is compared to the low/medium threshold and the medium/high threshold. If the process 500 determines that the numeric score exceeds the medium/high threshold, the process 500 can label the associated fusion as having a high likelihood of pathogenicity. If the process 500 determines that the numeric score exceeds the low/medium threshold but does not exceed the medium/high threshold, the process 500 can label the associated fusion as having a medium likelihood of pathogenicity. If the process 500 determines that the numeric score does not exceed the low/medium threshold, the process 500 can label the associated fusion as having a low likelihood of pathogenicity. Threshold values may be selected (e.g., chosen by a user) and/or received (e.g., a predetermined threshold). In one example, threshold values are selected according to the process 700 disclosed in FIG. 7.

In some embodiments, the process 500 can store numeric scores and/or categorizations generated by the trained classifier in a database of fusions and associated numeric scores and/or categorizations. The database may further include labeled fusion data (e.g., as described in FIGS. 8B and *D) and/or clinical data associated with the fusions.

Biomarkers can be informative relative to predicting a patient's diagnosis, prognosis, risk, response to therapy, or other possible wellness indicators. In some embodiments, the process 500 can be used in biomarker discovery by using trained classifier to rank fusion events, select a fusion event ranked with a high likelihood of pathogenicity that has not been previously shown to be pathogenic (for example, in a published scientific research article or other source of information), and designate the fusion as pathogenic. The process 500 may further include matching potential therapies, which may be matched based on the genes associated with each partner sequence in the fusion (e.g., if one of the genes is known to be targeted by the therapy), or other characteristics associated with the fusion. The process 500 may further include storing the fusion and associated data (e.g., labeled fusion data, clinical data, potential therapy matches etc.) in a database.

In some embodiments, the process 500 can be used in drug discover applications. In some embodiments, the process 500 can use the trained classifier to rank fusion events, select a fusion event ranked with a high likelihood of pathogenicity, grow a tumor organoid in vitro having the same or substantially similar fusion or using recombinant technologies to construct the fusion sequence and introduce the tumor organoid into an in vitro system (for example, a cell line or organoid line/model), observe the efficacy of cancer treating drugs in killing tumor/organoid cells having the fusion, report a list of effective drugs on a report for a patient having the fusion, and/or store the list of effective drugs and the associated fusion in a database. For example, an organoid may be genetically engineered to have the same characteristics as the specimen and may be observed after exposure to a therapy to determine whether the therapy can reduce the growth rate of the organoid, and thus may be likely to reduce the growth rate of the patient associated with the specimen. For example, organoids may be cultured and tested according to the systems and methods disclosed in U.S. patent application No. 16/693,117, titled “Tumor Organoid Culture Compositions, Systems, and Methods”, filed Nov. 22, 2019; U.S. Prov. Patent Application No. 62/924,621, titled “Systems and Methods for Predicting Therapeutic Sensitivity”, filed Oct. 22, 2019; U.S. Prov. Patent Application No. 62/944,292, titled “Large Scale Phenotypic Organoid Analysis”, filed Dec. 5, 2019; U.S. Prov. Patent Application No. 63/012,885, titled “Systems and Methods for High Throughput Drug Screening”, filed Apr. 20, 2020, and U.S. Patent Application No. 17/114,386, filed on Dec. 7, 2020, which are each incorporated herein by reference and in their entirety for all purposes.

In some embodiments, the process 500 can proceed to optional 535. At 535, the process 500 can recommend a fusion for review. In some embodiments, a fusion and the associated numeric score generated at 525 and/or pathogenicity likelihood category generated at 525 and/or 530 may be designated and/or delivered to a process including detailed manual review by an expert (for example, a trained variant scientist, board-certified clinical molecular geneticist, board-certified pathologist, etc.).

In some embodiments, the process 500 can recommend a fusion for review if the trained classifier output indicates that the fusion is likely to be pathogenic and the fusion's relevance to tumor biology and/or functional mechanism underlying pathogenicity is not known. For example, if the process 500 assigns high pathogenicity likelihood to a fusion, but one or gene(s) associated with the sequence partners of the fusion have not been previously associated with disease, the process 500 can recommend the fusion for review.

In some embodiments, the process 500 can recommend a fusion for review if the pathogenicity likelihood category assigned to the fusion at 530 is ambiguous. For example, if the difference between the numeric score associated with the fusion and the threshold value used to assign the risk category is smaller than a user-selected value or if the difference between the fusion characteristic (for example, breakpoint spanning reads) and the threshold value used to assign the risk category is smaller than a user-selected value.

In some embodiments, the process 500 can proceed to optional 540. At 540, the process 500 can recommend a fusion for biological validation. In some embodiments, if a fusion is categorized as likely to be pathogenic or if the risk category is ambiguous, as described above, the process 500 can recommend the fusion for biological validation. Biological validation may include experimental follow-up, validation using additional in-silico analyses (for example, analyzing the structure or stability of the protein associated with the fusion, analyzing the effect of the presence or absence of various protein domains on the protein associated with the fusion, analyzing the possible protein-protein interactions of the protein associated with the fusion, etc.) or wet-lab experiments (for example, introducing the fusion into an organoid or cell line and observing the effect on cell growth and cell health).

In some embodiments, the process 500 can proceed to optional 545. At 545, the process 500 can generate a fusion report. In some embodiments, the report can include the numeric score generated at 525 and/or pathogenicity likelihood category generated at 525 and/or 530. In some embodiments, the report cab include a pathogenicity likelihood, a qualitative pathogenicity category label, quantitative scores, and/or a driver score.

In some embodiments, the report can include one or more detected fusions based on the pathogenicity metrics. For example, the fusions can be selected from the at least one detected fusion based on the pathogenicity metrics. In some embodiments, the process 500 can select one or more detected fusions having a “high” and/or “medium” categorization to include in the report. In some embodiments, the report can include at least one therapy matched to the patient based on the detected fusions. For example, the process can identify (e.g., using a treatment database) one or more treatments used to treat patients having the detected fusions.

In some embodiments, the report can be a clinical report (for example, a patient report) that provides clinical support for personalized cancer therapy, using the information curated from sequencing of a liquid biopsy sample, as described above. In some embodiments, the report can be provided to a patient, physician, medical personnel, or researcher in a digital copy (for example, a JSON object, a pdf file, or an image on a website or portal), a hard copy (for example, printed on paper or another tangible medium). In some embodiments, a report object, such as a JSON object, can be used for further processing and/or display. For example, information from the report object can be used to prepare a clinical laboratory report for return to an ordering physician. In some embodiments, the report is presented as text, as audio (for example, recorded or streaming), as images, or in another format and/or any combination thereof.

The report can include information related to the specific characteristics of the patient's cancer, for example, cancer type, detected genetic variants, epigenetic abnormalities, associated oncogenic pathogenic infections, and/or pathology abnormalities. The report can include information related to detected gene fusions, especially gene fusions ranked likely to be pathogenic by the systems and methods, and other characteristics of a patient's sample and/or clinical records.

In some embodiments, other characteristics of a patient's sample and/or clinical records can also be included in the report. For example, in some embodiments, the clinical report includes information on clinical variants, for example, one or more of copy number variants (for example, for actionable genes CCNE1, CD274(PD-L1), EGFR, ERBB2(HER2), MET, MYC, BRCA1, and/or BRCA2), fusions, translocations, and/or rearrangements (for example, in actionable genes ALK, ROS1, RET, NTRK1, FGFR2, FGFR3, NTRK2 and/or NTRK3), pathogenic single nucleotide polymorphisms, insertion-deletions (for example, somatic/tumor and/or germ line/normal), therapy biomarkers, gene expression calls (for example, over- or under-expression of a gene compared to the expression level of that gene in normal tissue), microsatellite instability status, and/or tumor mutational burden.

In some embodiments, identified clinical variants are labeled as “potentially actionable”, “biologically relevant”, “variants of unknown significance (VUSs)”, or “benign”. Potentially actionable alterations are protein-altering variants with an associated therapy based on evidence from the medical literature. Biologically relevant alterations are protein-altering variants that may have functional significance or have been observed in the medical literature but are not associated with a specific therapy. Variants of unknown significance (VUSs) are protein-altering variants exhibiting an unclear effect on function and/or without sufficient evidence to determine their pathogenicity. In some embodiments, benign variants are not reported. In some embodiments, variants are identified through aligning the patient's DNA sequence to the human genome reference sequence version hg19 (GRCh37) or RNA sequence to the human genome reference sequence version GRCh38. In some embodiments, actionable and biologically relevant somatic variants are provided in a clinical summary during report generation.

For instance, in some embodiments, variant classification and reporting is performed, where detected variants are investigated following criteria from known evolutionary models, functional data, clinical data, literature, and other research endeavors, including tumor organoid experiments. In some embodiments, variants are prioritized and classified based on known gene-disease relationships, hotspot regions within genes, internal and external somatic databases, primary literature, and other features of somatic drivers. Variants can be added to a patient (or sample, for example, organoid sample) report based on recommendations from the Association for Molecular Pathology (AMP), American Society of Clinical Oncology (ASCO), or College of American Pathologists (CAP) guidelines. Additional guidelines may be followed (for example, National Comprehensive Cancer Network (NCCN) or Food and Drug Administration (FDA). Briefly, pathogenic variants with therapeutic, diagnostic, or prognostic significance may be prioritized in the report. Non-actionable pathogenic variants may be included as biologically relevant, followed by variants of uncertain significance. Translocations may be reported based on outputs from the trained classifier, features of known gene fusions, relevant breakpoints, and biological relevance. Evidence may be curated from public and private databases or research and presented as 1) consensus guidelines 2) clinical research, or 3) case studies, with a link to the supporting literature. Germline alterations may be reported as secondary findings in a subset of genes for consenting patients. These may include genes recommended by the ACMG and additional genes associated with cancer predisposition or drug resistance.

In some embodiments, a clinical report includes information about clinical trials for which the patient is eligible, matched therapies that are specific to the patient's cancer, and/or possible therapeutic adverse effects associated with the specific characteristics of the patient's cancer, for example, the patient's genetic variations, epigenetic abnormalities, associated oncogenic pathogenic infections, and/or pathology abnormalities, or other characteristics of the patient's sample and/or clinical records. For example, in some embodiments, the clinical report includes such patient information and analysis metrics, including cancer type and/or diagnosis, variant allele fraction, patient demographic and/or institution, matched therapies (for example, FDA approved and/or investigational), matched clinical trials, variants of unknown significance (VUS), genes with low coverage, panel information, specimen information, details on reported variants, patient clinical history, status and/or availability of previous test results, and/or version of bioinformatics pipeline.

In some embodiments, the report can include fusion matching information. In some embodiments, fusions associated with the gene ABL1 (where the fusions may also be associated with the BCR gene) in samples having acute lymphocytic leukemia or chronic myeloid leukemia cancer types may be matched with the following therapies: Dasatinib, Imatinib, Nilotinib, Ponatinib, or Bosutinib. Fusions associated with the gene ALK in samples having a number of cancer types (for example, Biliary Cancer, Bladder Cancer, Breast Cancer, Cervical Cancer, Chromophobe Renal Cell Carcinoma, Clear Cell Renal Cell Carcinoma, Endometrial Cancer, Esophageal Cancer, Gastric Cancer, Head and Neck Cancer, Head and Neck Squamous Cell Carcinoma, Liver Cancer, Low Grade Glioma, Melanoma, Meningioma, Non-Clear Cell Renal Cell Carcinoma, Non-Small Cell Lung Cancer, Oropharyngeal Cancer, Ovarian Cancer, Pancreatic Cancer, Retinoblastoma, Sarcoma, Testicular cancer, Thyroid Cancer, Kidney Cancer, or Skin Cancer) may be matched with the following therapies: alectinib, crizotinib, ceritinib, brigatinib, or lorlatinib. Fusions associated with the gene RSPO3 (where the fusions may be further associated with the PTPRK gene) in samples having a colorectal cancer type may be matched with the following therapies: Paclitaxel, or an RSPO3 Inhibitor (for example, Rosmantuzumab) (see, PMID: 29127379). Fusions associated with the gene FGFR2 in samples having a number of cancer types (for example, Breast Cancer, Cervical Cancer, Chromophobe Renal Cell Carcinoma, Clear Cell Renal Cell Carcinoma, Colorectal Cancer, Endometrial Cancer, Esophageal Cancer, Gastric Cancer, Head and Neck Cancer, Head and Neck Squamous Cell Carcinoma, Low Grade Glioma, Melanoma, Meningioma, Non-Clear Cell Renal Cell Carcinoma, Non-Small Cell Lung Cancer, Oropharyngeal Cancer, Ovarian Cancer, Pancreatic Cancer, Retinoblastoma, Sarcoma, Testicular cancer, Thyroid Cancer, Kidney Cancer, Skin Cancer, Glioblastoma, Tumor of Unknown Origin) may be matched with the following therapies: erdafitinib, pemigatinib. Fusions associated with the gene FGFR2 in samples having liver cancer or biliary cancer types may be matched with the following therapies: erdafitinib, derazantinib, pemigatinib, ponatinib, or infigratinib. Fusions associated with the gene FGFR2 in samples having a bladder cancer type may be matched with the following therapies: erdafitinib. Fusions associated with the gene ERG (where the fusions may also be associated with the TMPRSS2 gene) in samples having a prostate cancer type may be matched with the following therapies: PARP inhibitor with radiation. Fusions associated with the gene ROS1 in samples having a non-small cell lung cancer cancer type may be matched with the following therapies: crizotinib, ceritinib, lorlatinib, entrectinib, or cabozantinib. Fusions associated with the gene BRAF in samples having a melanoma cancer type may be matched with the following therapies: trametinib. Fusions associated with the gene FGFR1 (where the fusions may also be associated with the FN1 gene) in samples having a sarcoma cancer type may be matched with the following therapies: pazopanib. Fusions associated with the gene MYB in samples having a head and neck cancer type may be matched with the following therapies: regorafenib, or gefitinib; crizotinib; linsitinib. Fusions associated with the gene RET (where the fusions may also be associated with the NCOA4 gene) in samples having a thyroid cancer type may be matched with the following therapies: cabozantinib, vandetanib, sunitinib, nintedanib, lenvantinib, vandetanib, or ponatinib. Fusions associated with the gene MET in samples having a glioblastoma cancer type may be matched with the following therapies: foretinib. Fusions associated with the gene NRG1 in samples having a non-small cell lung cancer type may be matched with the following therapies: afatinib, or anti-HER3 monoclonal antibody (MAb). Fusions associated with the gene ALK in samples having a colorectal cancer type may be matched with the following therapies: ceritinib, or entrectinib. Fusions associated with the genes NTRK1, NTRK2, or NTRK3 in samples having brain cancer, glioblastoma, or low grade glioma cancer types may be matched with the following therapies: ropotrectinib, larotrectinib, or entrectinib. Fusions associated with the genes NTRK1, NTRK2, or NTRK3 in samples having a number of cancer types (for example, Biliary Cancer, Bladder Cancer, Breast Cancer, Cervical Cancer, Chromophobe Renal Cell Carcinoma, Clear Cell Renal Cell Carcinoma, Endometrial Cancer, Esophageal Cancer, Gastric Cancer, Head and Neck Cancer, Head and Neck Squamous Cell Carcinoma, Liver Cancer, Low Grade Glioma, Melanoma, Meningioma, Non-Clear Cell Renal Cell Carcinoma, Oropharyngeal Cancer, Ovarian Cancer, Pancreatic Cancer, Retinoblastoma, Sarcoma, Testicular cancer, Thyroid Cancer, Kidney Cancer, Skin Cancer, Prostate Cancer) may be matched with the following therapies: ropotrectinib, larotrectinib, or entrectinib. Fusions associated with the gene TACC3 (where the fusions may also be associated with the FGFR3 gene) in samples having a glioblastoma cancer type may be matched with the following therapies: erdafitinib, or ponatinib. Fusions associated with the gene NRG1 in samples having a number of cancer types (for example, Biliary Cancer, Bladder Cancer, Breast Cancer, Cervical Cancer, Chromophobe Renal Cell Carcinoma, Clear Cell Renal Cell Carcinoma, Colorectal Cancer, Endometrial Cancer, Esophageal Cancer, Gastric Cancer, Head and Neck Cancer, Head and Neck Squamous Cell Carcinoma, Liver Cancer, Low Grade Glioma, Melanoma, Meningioma, Non-Clear Cell Renal Cell Carcinoma, Oropharyngeal Cancer, Ovarian Cancer, Pancreatic Cancer, Retinoblastoma, Sarcoma, Testicular cancer, Thyroid Cancer, Kidney Cancer, or Skin Cancer) may be matched with the following therapies: afatinib, an anti-HER3 MAb, or a combination of erlotinib+pertuzumab. Fusions associated with the gene RET in samples having a non-small cell lung cancer type may be matched with the following therapies: cabozantinib, vandetanib, sunitinib, RET Inhibitor (for example, LOXO-292), nintedanib, or lenvatinib. Fusions associated with the gene TFE3 (where the fusions may also be associated with the ASPL gene) in samples having a sarcoma cancer type may be matched with the following therapies: sunitinib, pazopanib, cabozantinib, or dasatinib. Fusions associated with the gene BRAF in samples having a sarcoma cancer type may be matched with the following therapies: regorafenib. Fusions associated with the gene MYH11 (where the fusions may also be associated with the CBFB gene) in samples having an acute myeloid leukemia cancer type may be matched with the following therapies: fludarabine; cytarabine; idarubicin; gemtuzumab ozogamicin, or entinostat. Fusions associated with the gene ARHGAP26 (where the fusions may also be associated with the CLDN18 gene) in samples having a gastric cancer type may be matched with the following therapies: platinum agents, or fluorouracil (5-FU). Fusions associated with the gene EGFR in samples having a glioblastoma or melanoma cancer type may be matched with the following therapies: EGFR inhibitors, or cetuximab. Fusions associated with the gene EGFR in samples having a glioblastoma or non-small cell lung cancer type may be matched with the following therapies: afatinib with temozolomide or nimotuzumab with temozolomide. Fusions associated with the gene EGFR (where the fusions may also be associated with the SEPT14 gene) in samples having a glioblastoma or brain cancer type may be matched with the following therapies: erlotinib or lapatinib. Fusions associated with the gene ESR1 in samples having a number of cancer types (for example, Biliary Cancer, Bladder Cancer, Breast Cancer, Cervical Cancer, Chromophobe Renal Cell Carcinoma, Clear Cell Renal Cell Carcinoma, Colorectal Cancer, Endometrial Cancer, Esophageal Cancer, Gastric Cancer, Head and Neck Cancer, Head and Neck Squamous Cell Carcinoma, Liver Cancer, Low Grade Glioma, Melanoma, Meningioma, Non-Clear Cell Renal Cell Carcinoma, Non-Small Cell Lung Cancer, Oropharyngeal Cancer, Ovarian Cancer, Pancreatic Cancer, Retinoblastoma, Sarcoma, Testicular cancer, Thyroid Cancer, Kidney Cancer, Skin Cancer, or Prostate Cancer) may be matched with the following therapies: palbociclib, fulvestrant, or tamoxifen; and may be accompanied with a note in the report that these fusions may be associated with resistance to aromatase inhibitors.

In some embodiments, the report can include one or more fusions selected to aid a physician in diagnosis and additional medical decision making. For example, the following diagnostic fusions may be reported: fusions associated with the gene STAT6 (where the fusions may also be associated with the NAB2 gene) in samples having a sarcoma cancer type, fusions associated with the gene ERG (where the fusions may also be associated with the TMPRSS2 gene) in samples having a prostate cancer type, fusions associated with the gene PLAG1 in samples having sarcoma or head and neck cancer types, fusions associated with the gene MYB in samples having a head and neck cancer type, fusions associated with the gene CCNB3 (where the fusions may also be associated with the BCOR gene) in samples having a sarcoma cancer type, fusions associated with the gene MAML2 (where the fusions may also be associated with the CRTC1 gene) in samples having a head and neck cancer type, fusions associated with the gene TFE3 in samples having clear cell renal cell carcinoma or non-clear cell renal cell carcinoma cancer types, fusions associated with the gene RELA (where the fusions may also be associated with the C11orf95 gene) in samples having brain cancer or low grade glioma cancer types, fusions associated with the gene TFE3 (where the fusions may also be associated with the ASPL gene) in samples having a sarcoma cancer type, fusions associated with the gene ABL1 (where the fusions may also be associated with the BCR gene) in samples having a chronic myeloid leukemia cancer type, fusions associated with the gene PRKACA (where the fusions may also be associated with the DNAJB1 gene) in samples having a liver, pancreas, or biliary cancer type, and fusions associated with the gene PRKACA (where the fusions may also be associated with the DNAJB2 gene) in samples having a liver cancer type. Various prognostic fusions may be reported with a matched prognosis. Fusions associated with the gene MET in samples having a glioblastoma cancer type may be matched with an unfavorable prognosis. Fusions associated with the gene TFE3 in samples having clear cell renal cell carcinoma or non-clear cell renal cell carcinoma cancer types may be matched with an unfavorable prognosis. Fusions associated with the gene AFF1 (where the fusions may also be associated with the MLL1 gene) in samples having an acute lymphocytic leukemia cancer type may be matched with an unfavorable prognosis. Fusions associated with the gene RELA (where the fusions may also be associated with the C11orf95 gene) in samples having brain cancer or low grade glioma cancer types may be matched with an unfavorable prognosis. Fusions associated with the gene TACC3 (where the fusions may also be associated with the FGFR3 gene) in samples having a glioblastoma cancer type may be matched with a favorable prognosis. Fusions associated with the gene ARHGAP26 (where the fusions may also be associated with the CLDN18 gene) in samples having a gastric cancer type may be matched with an unfavorable prognosis.

Various risk fusions may be reported with a matched risk. Fusions associated with the gene MYH11 (where the fusions may also be associated with the CBFB gene) in samples having an acute myeloid leukemia cancer type may be matched with a favorable risk.

In some embodiments, the results included in the report, and/or any additional results (for example, from the bioinformatics pipeline), can be used to query a database of clinical data, for example, to determine whether there is a trend showing that a particular therapy was effective or ineffective in treating (for example, slowing or halting cancer progression), and/or adverse effects of such treatments in other patients having the same or similar characteristics.

In some embodiments, the results are used to design cell-based studies of the patient's biology, for example, tumor organoid experiments. For example, an organoid may be genetically engineered to have the same characteristics as the specimen and may be observed after exposure to a therapy to determine whether the therapy can reduce the growth rate of the organoid, and thus may be likely to reduce the growth rate of the cancer in the patient associated with the specimen. Similarly, in some embodiments, the results are used to direct studies on tumor organoids derived directly from the patient. An example of such experimentation is described in U.S. Provisional Patent Application No. 62/944,292, filed Dec. 5, 2019, the content of which is hereby incorporated by reference, in its entirety, for all purposes.

In some embodiments, the report can be checked for final validation, review, and sign-off by a medical practitioner (for example, a pathologist). The clinical report is then sent for action (for example, for precision oncology applications).

In some embodiments, the process 500 can proceed to optional 550. At 550, the process 500 can output the report. The process 500 can output the report and/or fusions to a physician, a medical personnel, and/or a patient to guide medical care of the patient.

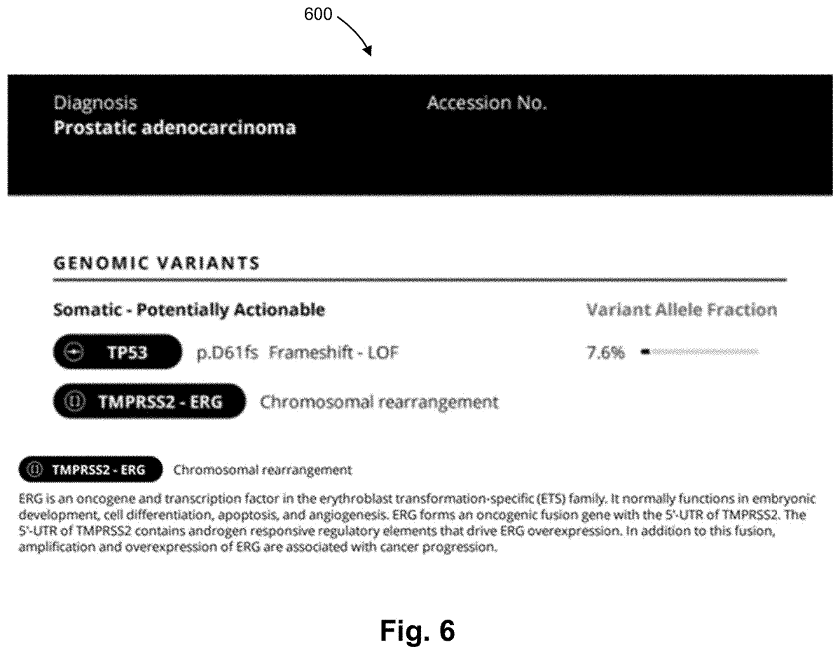

FIG. 6 illustrates an example clinical report 600. In the report 600, the patient's diagnosis is prostatic adenocarcinoma and the clinical report includes a variant report and description with canonical fusion (TMPRSS2-ERG) prioritized as ‘high’ likelihood of pathogenicity by a trained classifier and a TP53 p.D61fs frameshift loss of function variant.

FIG. 7 illustrates an exemplary process 700 to train a classifier. The process 700 can be implemented as computer readable instructions on one or more memories or other non-transitory computer readable media, and executed by one or more processors in communication with the one or more memories or other media. In some embodiments, the process 700 can be implemented as computer readable instructions on the persistent memory 112 and/or the non-persistent memory 111 and executed by the processor 102.

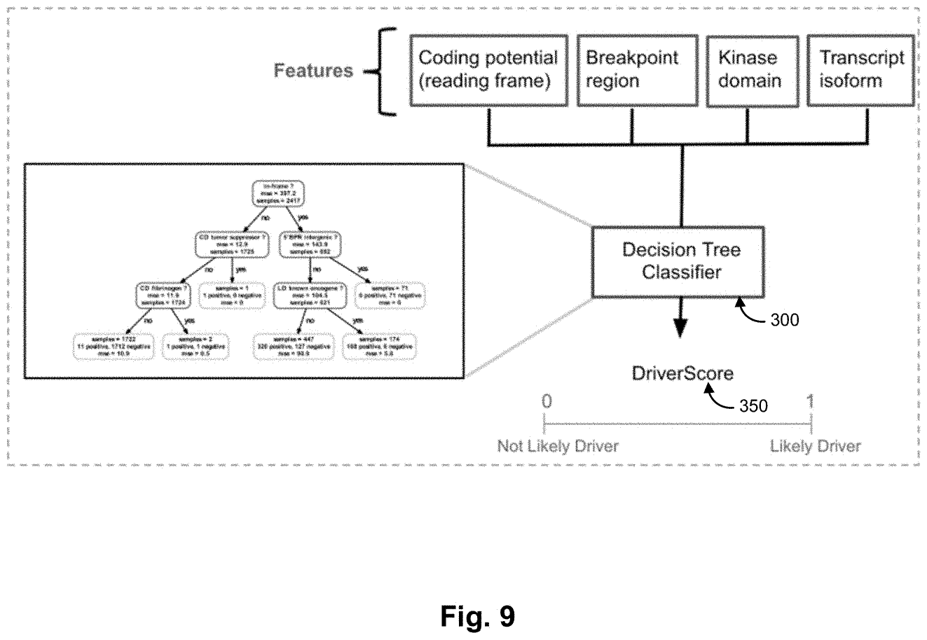

At step 705, the process 700 can select at least one classifier. In one embodiment, the classifier may include a decision tree classifier (e.g., as shown in FIG. 9). In some embodiments, the classifier can include a gradient boosting model, a random forest model, a neural network (NN), a regression model, a Naive Bayes models, and/or machine learning algorithms (MLA). A MLA or a NN can be trained from a training data set such as a plurality of matrices having a feature vector for each patient or images and features. In some embodiments, a training data set may include imaging, pathology, clinical, and/or molecular reports and details of a patient, such as those curated from an EHR or genetic sequencing reports. The training data may be generated based on features such as the objective specific sets disclosed herein. MLAs include supervised algorithms (such as algorithms where the features/classifications in the data set are annotated) using linear regression, logistic regression, decision trees, classification and regression trees, Naïve Bayes, nearest neighbor clustering; unsupervised algorithms (such as algorithms where no features/classification in the data set are annotated) using Apriori, means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as mincut, harmonic function, manifold regularization), heuristic approaches, or support vector machines. NNs can include conditional random fields, convolutional neural networks, attention based neural networks, deep learning, long short term memory networks, and/or other neural models where the training data set includes a plurality of tumor samples, RNA expression data for each sample, and pathology reports covering imaging data for each sample. While MLA and neural networks identify distinct approaches to machine learning, the terms may be used interchangeably herein. Thus, a mention of MLA may include a corresponding NN, or a mention of NN may include a corresponding MLA unless explicitly stated otherwise.

At 710, the process 700 can select training data. In some embodiments, the process 700 can select a group of positive control fusions and a group of negative control fusions. The positive control fusions may be fusions that are canonical or known to be pathogenic. The negative control fusions may be fusions that are found in healthy or normal tissue, indicating that the fusions are not likely to cause disease. For each group, the process 700 can select fusions such that each possible variety (or a sufficiently representative proportion or majority of possible varieties) of a given feature (e.g., strandedness) is represented among the group. See example below for selection of positive controls based on all possible combinations of strandedness.

The training data can include selected positive control fusions, negative control fusions, and/or data associated with these fusions. For example, the training data can include any data types selected as features.

In some embodiments, the features can include at least one of reading frame status such as yes/no, in frame/out of frame, and/or 0 or 1, breakpoint regions such as a positive integer relating to a genomic coordinate, transcript isoform (e.g., entries including string labels such as NCBI RefSeq ID or Ensembl ID), and kinase (e.g., entries including yes/no, intact/disrupted, etc.), or other features relevant to genomic annotation information pertaining to fusion event, conserved/lost domains and motifs, for capturing pertinent functional features of the fusion protein product, particularly in regards to oncogenic activity vs tumor suppressor activity (e.g., entries including string labels such as Pfam/InterPro IDs for domains and Smart for motifs), target breakpoint regions or ‘ROI’, for prioritizing fusion events in which breakpoint(s) is contained within a defined target breakpoint, region or fusion hotspot (possible entries may include range of positive integers), read support, (e.g., entries including a positive integer, number of high quality reads spanning breakpoint) for discerning between real vs. technical artifacts, actionability, (for example, druggability of the fusion or another medical treatment decision associated with the fusion, which may be based on therapeutic, prognostic or diagnostic context), for prioritizing fusion events which are therapeutically actionable and which can further be weighted by evidence level (e.g., entries including a string label indicating internal curation of actionability in internal database), cancer type, for prioritizing fusion events which are canonical to specific tumor types (e.g., entries including a categorical string label), RNA and DNA concordance, for indicating if a fusion event is observed in both DNA and RNA (e.g., entries including yes/no, DNA/RNA/both), pathway (protein-protein interactions, metabolites), for using network centrality scoring which has been used previously for fusion prioritization (See Wu et al, Bioinformatics. 2013 May 1; 29(9):1174-81, which is incorporated by reference herein in its entirety) or a domain-based network approach (Wu et al, PLoS Comput Biol. 2018 Jul. 24; 14(7):e1006266, which is incorporated by reference herein in its entirety) (e.g., entries including a categorical string label), a mechanism (e.g., a biological mechanism explaining a possible connection between the fusion and an effect on the cancer associated with the sample)/functional evidence or functional ontology, for discerning whether loss of function (LOF) or GOF mechanism applies (e.g., entries including a categorical string label), canonical status of a fusion, for example a fusion may have canonical status based on consensus standards from NCCN or expert evaluation that may be documented in published literature or public or private databases (e.g., entries including yes/no), canonical status of breakpoints, for example, breakpoints in a fusion candidate may be documented or known to be present in pathogenic fusions (e.g., entries including yes/no), recurrence or frequency, for example, the presence of the fusion on previous clinical reports and/or in databases including Catalog of Somatic Mutations in Cancer (COSMIC), The Cancer Genome Atlas (TCGA), or detected and/or reported by other tumor mutation profiling tests (possible entries may be yes/no, a numeric value representing total number of times a fusion is observed versus expected, which may be determined by statistical methods, and/or the percent of cases having the fusion), and/or RNA expression, for determining whether RNA expression levels support fusion mechanism of action (such as overexpression of ERBB2 seen in tumor with ERBB2 fusion) (e.g., entries including a float numeric representing RNA-Seq expression value).

At 715, the process 700 can train at least one classifier. In some embodiments, the process 700 can include providing datasets as a matrix of feature vectors for each fusion, labeling these fusions as pathogenic (positive control) or passenger/artifact (negative control) as supervisory signals, and training the MLA to predict an objective/target pairing. During training, some MLA may identify features of importance and identify a coefficient, or weight, to associate with a feature of importance. The coefficient may be multiplied with the occurrence frequency of the feature to generate a score, and once the scores of one or more features exceed a threshold, certain classifications may be predicted by the MLA. A coefficient schema may be combined with a rule based schema to generate more complicated predictions, such as predictions based upon multiple features. For example, ten key features may be identified across different classifications. A list of coefficients may exist for the key features, and a rule set may exist for the classification. A rule set may be based upon the number of occurrences of the feature, the scaled weights of the features, or other qualitative and quantitative assessments of features encoded in logic known to those of ordinary skill in the art. In other MLA, features may be organized in a binary tree structure. For example, key features which distinguish between the most classifications may exist as the root of the binary tree and each subsequent branch in the tree until a classification may be awarded based upon reaching a terminal node of the tree. For example, a binary tree may have a root node which tests for a first feature. The occurrence or non-occurrence of this feature must exist (the binary decision), and the logic may traverse the branch which is true for the item being classified. Additional rules may be based upon thresholds, ranges, or other qualitative and quantitative tests.

In some embodiments, the classifier can include a decision tree, and the weight values may be determined by manual methods. In some embodiments, the classifier may include a machine learning algorithm and may have statistically determined weight values (for example, weight values may be determined by methods that include back propagation, loss function minimization, etc.)

At 720, the process 700 can generate at least one numeric score for each fusion in the training data. Multiple types of numeric scores may be generated for a fusion. In some embodiments, a larger value numeric score represents that the fusion is more likely to be pathogenic than a fusion that receives a smaller value numeric score. In some embodiments, the numeric score is a number in the range of 0 to 1.

At 725, the process 700 can select a threshold value for each type of numeric score and/or fusion characteristic and assigns qualitative pathogenicity risk classifications for each fusion in the training data and/or a holdout set of data. The process 700 can iteratively select threshold values based on each fusion.

In some embodiments, the possible qualitative pathogenicity risk classifications include low, medium, high. In another example, the possible qualitative pathogenicity risk classifications include benign, likely benign, unknown, likely pathogenic, and pathogenic.

In some embodiments, the process 700 can include assigning a qualitative pathogenicity classification for each fusion by include comparing a numeric score to a threshold value and if the numeric score exceeds the threshold value, assigning one of the qualitative pathogenicity classifications.

In some embodiments, the process 700 can compare a first threshold value to the numeric score generated by classifier and a second threshold value to a fusion characteristic. If both the numeric score and the number of fusion characteristic value exceed their respective thresholds, the associated fusion can be labeled as having a high likelihood of pathogenicity. If only one of either the numeric score or the fusion characteristic value exceeds its respective threshold, the associated fusion can be labeled as having a medium likelihood of pathogenicity. If neither the numeric score nor the fusion characteristic value exceed their respective thresholds, the associated fusion is labeled as having a low likelihood of pathogenicity. In one example, the threshold value for the numeric score is 0.5. In another example, the threshold value for the numeric score is 0.2. In another example, the threshold value for the numeric score is 0.8. In one example, the fusion characteristic is the number of high quality reads spanning the breakpoint and the fusion characteristic threshold is forty. Many factors may influence the value selected for a threshold, including changes in sequencing coverage (for example, due to a change or difference in the panel used to sequence the sample), changes in upstream fusion detection tools, new assay validation data, etc.

In some embodiments, values for one or more threshold types may be selected arbitrarily. In some embodiments, values for one or more threshold types may be selected by testing the effect of multiple values for a particular threshold type on the trained classifier performance. Assessing the effect on the trained classifier performance may include comparing the generated numeric scores and/or pathogenicity class for each fusion to the fusion's known status as a driver (positive control) or passenger (negative control) and may further include calculating performance metrics at 735.

In some embodiments, the process 700 can proceed to optional 735. At 735, the process 700 can calculate performance metrics. In one example, performance metrics may include sensitivity, specificity, false negative rate, false positive rate, receiver operating characteristic (ROC), area under curve (AUC), etc. In one example, threshold values are selected based on performance metrics calculated for classifier. In this example, for a particular threshold type, multiple threshold values are selected and for each threshold value performance metrics are calculated for the classifier. In some embodiments, the threshold value associated with the optimal performance metrics is selected. The performance metrics calculated at 735 may also serve as a validation of the classifier. In some embodiments, the process can subsequently execute 720 through 735 with fusion data that were not selected for training at step 715.

### EXAMPLES

Exemplary Scoring/Categorization Pipeline.

FIG. 8A displays a specific exemplary pipeline 800 for fusion scoring and categorization using methods identified with respect to FIG. 5. At a step 1, a bioinformatics database may be queried to pull processed fusion data (either RNA or DNA fusions) for a given sample. In another example, a software tool called AGFusion may also label the fusion based on various properties/features (for example, reading frame, domains, etc). FIG. 8B displays exemplary RNA fusion data for a single case processed by a bioinformatics pipeline and stored in an ‘rna_fusion’ database table.

This data serves as starting input data for step 1 shown in FIG. 8A. The table contains the following fields: run_id: sample- and workflow-specific identifier generated by Bioinformatics pipeline (not shown); gene_name_5: gene symbol (as per HUGO Gene Nomenclature Committee (HGNC)) for 5′ gene partner; gene_name_3: gene symbol (as per HUGO Gene Nomenclature Committee (HGNC)) for 3′ gene partner; chr_5: human chromosome on which 5′ gene partner is located; chr_3: human chromosome on which 3′ gene partner is located; breakpoint_5: genomic coordinate where fusion junction occurs in the 5′ gene (In this example coordinates are in accordance with human reference genome build GRCh37 (hg19) for DNA and GRCh38 (hg38) for RNA); breakpoint_3: genomic coordinate where fusion junction occurs in the 3′ gene (In this example coordinates are in accordance with human reference genome build GRCh37 (hg19) for DNA and GRCh38 (hg38) for RNA; spanning_breaks: number of reads spanning fusion breakpoint; and hq_spanning_breaks: total number of high quality reads supporting fusion.

At a step 2, a converter script may be run to process and annotate each fusion candidate for scoring. This may involve performing a liftover of hg38 genomic coordinates to hg19 genomic coordinates (it is noted that this only applies for RNA fusions which are in hg38 but not DNA fusions which are already in hg19), annotating 5′ and 3′ gene partners genomic start/end and breakpoints based on strandedness and strand pairings (for example, for each strand, is the strand a forward (+) or reverse (−) strand, are both partners forward (++) strands, are both reverse (−−) strands, is the 5′ forward and the 3′ reverse (+−), and/or is the 5′ reverse and the 3′ forward (−+)) based on the logic outlined below in FIG. 8C. For DNA fusions, an additional conversion may be made in which intronic breakpoints are converted to the closest exon boundary upstream (for a 5′ partner) or downstream (for a 3′ partner). The most clinically relevant transcript isoform exon structure is used based on a curated database of annotated transcript isoforms. This conversion allows for determination of reading frame potential for DNA fusions but is not needed for RNA fusions as intronic breakpoints are not possible in RNA fusions.

FIG. 8C displays exemplary logic for annotation of genomic start/end and breakpoints for 5′ and 3′ gene partners based on gene's strandedness, which may be used to allow for appropriate reconstruction of chimeric fusion sequence by the fusion predictor in step 3.

FIG. 8D displays RNA fusion data after having been converted and labeled. The fields are 5p_symbol, for the gene symbol (as per HUGO Gene Nomenclature Committee (HGNC)) for 5′ gene partner; 5p_ensembl, for the Ensembl ID for 5′ gene partner; 5p_strand, for the strandedness of 5′ gene partner (either +or −); 5p_chr, for the human chromosome on which 5′ gene partner is located; 3p_symbol, for the gene symbol (as per HUGO Gene Nomenclature Committee (HGNC)) for 3′ gene partner; 3p_ensembl, for the Ensembl ID for 3′ gene partner; 3p_strand, for the strandedness of 3′ gene partner (either +or −); 3p_chr, for the human chromosome on which 3′ gene partner is located; 5p_start, for the genomic coordinate start of 5′ gene partner sequence; 5p_end, for the genomic coordinate end of 5′ gene partner sequence; 3p_start, for the genomic coordinate start of 3′ gene partner sequence; 3p_end, for the genomic coordinate end of 3′ gene partner sequence; split_reads, for the number of reads spanning fusion breakpoint (spanning_breaks from FIG. 8B); tot_reads, for the total number of high quality reads supporting RNA fusion (hq_spanning_breaks from FIG. 8B).

In step 3, the general input file generated in step 2 may be provided to a classifier 300. Various classifiers may be utilized in the operation of the classifier 300. One exemplary classifier, as shown in FIG. 8A, is Pegasus. Pegasus uses a machine learning approach to discern driver fusion events from artifactual or passenger fusion events by performing a) chimeric transcript sequence reconstruction and analysis (including coding frame, kinase and domain annotation), b) applying a decision tree classification algorithm with gradient boosting and c) generating a DriverScore metric to score the likeliness of a fusion candidate being a driver fusion event. This allows for automated fusion annotation and prioritization in a manner that is informed by a scoring metric. Other fusion classifiers in the oncology field are based on different machine-learning methods or different features. Specifically, the classifier 300 is based on the biological and technical features outlined in FIG. 5. FIGS. 8E-F collectively illustrate an exemplary output file resulting from step 3 of the fusion classifier pipeline.

A brief explanation of all fields are provided below:
DriverScore: scoring metric generated by classifier
FusionID: fusion identifier for each fusion candidate scored
Sample_Name: sample identifier
Program: denotes type of input file used (ie ‘general’ program is always used)
Tot/span_reads: total number of high quality reads supporting RNA fusion (hq_spanning_breaks from FIG. 8B)
Split_reads: number of reads spanning fusion breakpoint (spanning_breaks from FIG. 8B)
Chr1: human chromosome on which 5′ gene partner is located
Chr2: human chromosome on which 3′ gene partner is located
Gene_Start1: genomic coordinate start of 5′ gene partner sequence
Gene_End1: genomic coordinate end of 5′ gene partner sequence
Gene_Start2: genomic coordinate start of 3′ gene partner sequence
Gene_End2: genomic coordinate end of 3′ gene partner sequence
Strand1: strandedness of 5′ gene partner (either +or −)
Strand2: strandedness of 3′ gene partner (either +or −)
Gene_Name1: gene symbol (as per HUGO Gene Nomenclature Committee (HGNC)) for 5′ gene partner
Gene_Name2: gene symbol (as per HUGO Gene Nomenclature Committee (HGNC)) for 3′ gene partner
Gene_Breakpoint1: genomic coordinate where fusion junction occurs in the 5′ gene
Gene_Breakpoint2: genomic coordinate where fusion junction occurs in the 3′ gene
Gene_ID1: Ensembl ID for 5′ gene partner
Gene_ID2: Ensembl ID for 3′ gene partner
Sample_Type: tissue type analyzed (always set to ‘tumor tissue’)
Sample_Occupancy: N/A
Sample_Occupancy_Type: N/A
Kinase_info: label for presence of kinase domain in either 5′ gene partner (‘5p_KINASE’), 3′ gene partner (‘3p_KINASE’) or both (‘BOTH’)
Transcript_ID1: Ensembl transcript isoform ID for 5′ gene partner
Transcript_ID2: Ensembl transcript isoform ID for 3′ gene partner
Reading_Frame: nucleotide reading frame of fusion transcript sequence (denotes protein coding potential; can be either ‘inFrame’ or ‘Frameshift’)
Protein_Start1: starting amino acid position encoded by 5′ gene partner sequence
Protein_End1: last amino acid position encoded by 5′ gene partner sequence
Protein_Start2: starting amino acid position encoded by 3′ gene partner sequence
Protein_End2: last amino acid position encoded by 3′ gene partner sequence
Protein_Sequence: protein amino acid sequence encoded by fusion transcript sequence (note: using single letter amino acid code)
Exon_Gene1: exon number of 5′ gene transcript sequence in which breakpoint occurs
Exon_Gene2: exon number of 3′ gene transcript sequence in which breakpoint occurs
Breakpoint_Region1: region of 5′ gene in which breakpoint occurs (can be either in coding (CDS) sequence or 5′UTR (5′ untranslated region) or 3′UTR (3′ untranslated region)
Breakpoint_Region2: region of 3′ gene in which breakpoint occurs (can be either in coding (CDS) sequence or 5′UTR (5′ untranslated region) or 3′UTR (3′ untranslated region)
Conserved_Domain1: Preserved domain(s) of 5′ gene partner
Lost_Domain1: Lost domain(s) of 5′ gene partner
Conserved_Domain2: Preserved domain(s) of 3′ gene partner
Lost_Domain2: Preserved domain(s) of 3′ gene partner

In Step 4, output file generated by step 3 may be loaded to a web application which a) categorizes all scored fusion candidates based on DriverScore and read support levels and b) provides a user interface to view the results.

### Example: Exemplary Classifier

FIG. 9 illustrates various details of the classifier 300 described in FIG. 8. The fusion classifier method uses a set of biological features from the fusion (comprised of reading frame, breakpoint region, presence/absence of kinase domain and transcript isoform of gene fusion partners) in a binary decision tree classification algorithm in order to generate a scoring metric known as the DriverScore 350. The DriverScore ranges from 0 (denoting a fusion candidate which is not likely to be a driver fusion event in the tumor) to 1 (denoting a fusion candidate which is likely to be a driver fusion event in the tumor).

### Example: Fusion Categorization Plot

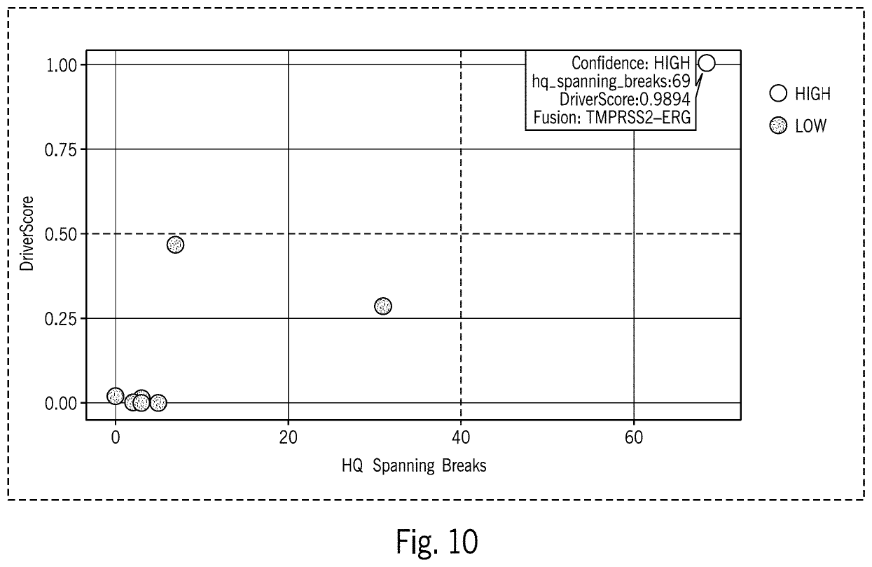

FIG. 10 illustrates a plot visualizing the categorization of fusions described in FIG. 8A. The plot shows the number of HQ spanning breaks (x-axis) and the output of fusions classifier 300 (y-axis) associated with each fusion (circle) and the thresholds (dotted lines) used to categorize each fusion as low (lower left quadrant), medium (upper left or lower right quadrants), or high (upper right quadrant). Specifically, FIG. 10 displays exemplary user interface results returned by web-based application of fusion classifier pipeline in which user can input a sample identifier to obtain profile of all fusion candidates scored and categorized by fusion classifier. Specifically, all fusion events are binned into High, Medium and Low Confidence categories based on DriverScore (DriverScore threshold of 0.5 is used) and read support (total read support of 40 is used) for each fusion. These thresholds were determined based on testing of internal positive and negative control fusion data. The example output shown above is from a prostate cancer case in which a well-established driver fusion, TMPRSS2-ERG has been prioritized into the High Confidence category (shown as the pink dot in the upper right quadrant of the plot) while the other fusion candidates detected in the sample scored in the Low Confidence category (shown as the blue dots in the lower left quadrant of the plot). The dotted lines denote DriverScore and read support thresholds (0.5 and 40, respectively).

### Further Examples

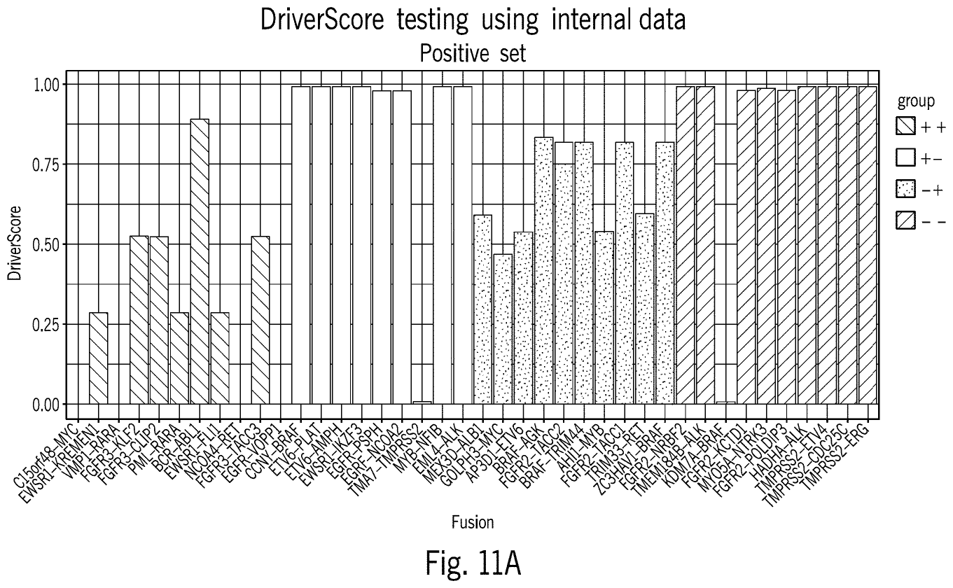

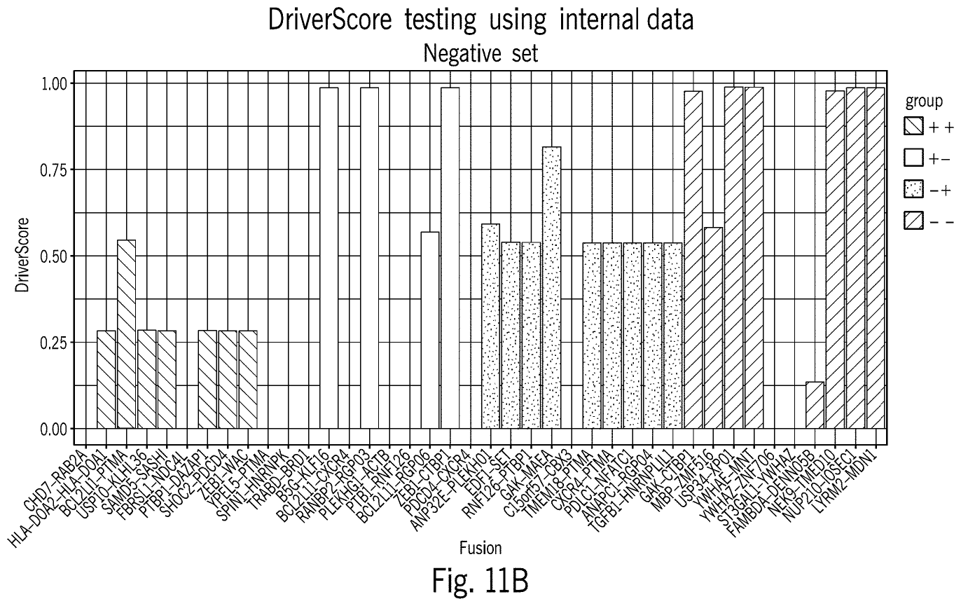

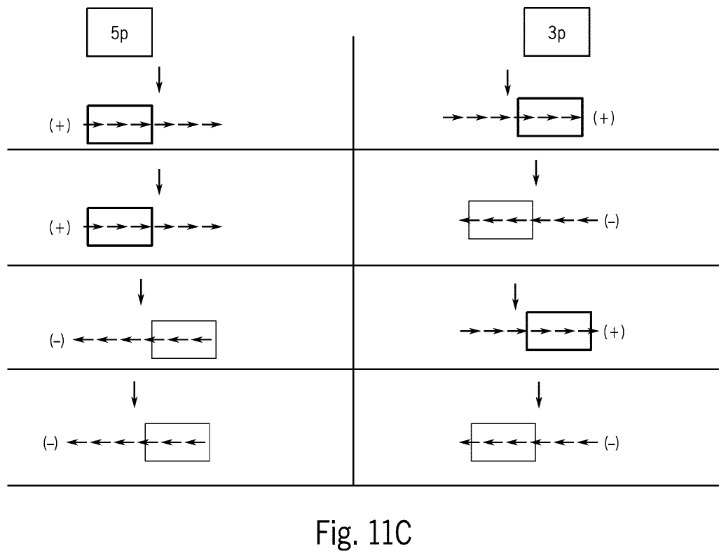

FIGS. 11A and 11B illustrate DriverScore testing using internal RNA fusion data as positive and negative test sets, respectively. The positive test set was comprised of well-established (canonical) driver fusions in tumors which are reportable. The negative test set was comprised of known non-driver fusions detected in normal tissues. Within each set, fusions were grouped based on the strandedness of the 5′ and 3′ gene partners (++, +−, −+, −−) to ensure that all possible strand combinations would be tested. A schematic depiction of the possible strand combinations for the 5′ and 3′ gene partners is shown on the right (red arrow indicates breakpoint, colored box indicates exon, gray line indicates intron). FIGS. 11A and 11B illustrate an example of positive and negative control testing sets used to calculate performance metrics for a trained classifier 300. FIG. 11C illustrates each possible strand status of each partner sequence.

Example: Selection of DriverScore thresholds. DriverScore thresholds were determined from the output of DriverScore testing using internal RNA fusion data as positive and negative test sets (top and bottom bar plots on the left). Performance metrics including sensitivity (purple line), specificity (green line), false negative rate (blue line) and false positive rate (red line) were calculated across the numeric range for DriverScore (0-1) and plotted (right hand side). Various DriverScore thresholds were set for the use in different contexts. A clinical threshold (0.2), which was derived for potential use in clinical workflow, provided highest sensitivity for capture of known driver fusions (in positive set). A Research & Development (R&D) threshold (0.8), which could be used for R&D-based implementation, provided the highest specificity for capture of all potential driver fusion events. A balanced threshold (0.5) which provides a balance of sensitivity and specificity was also determined for use in dual contexts.

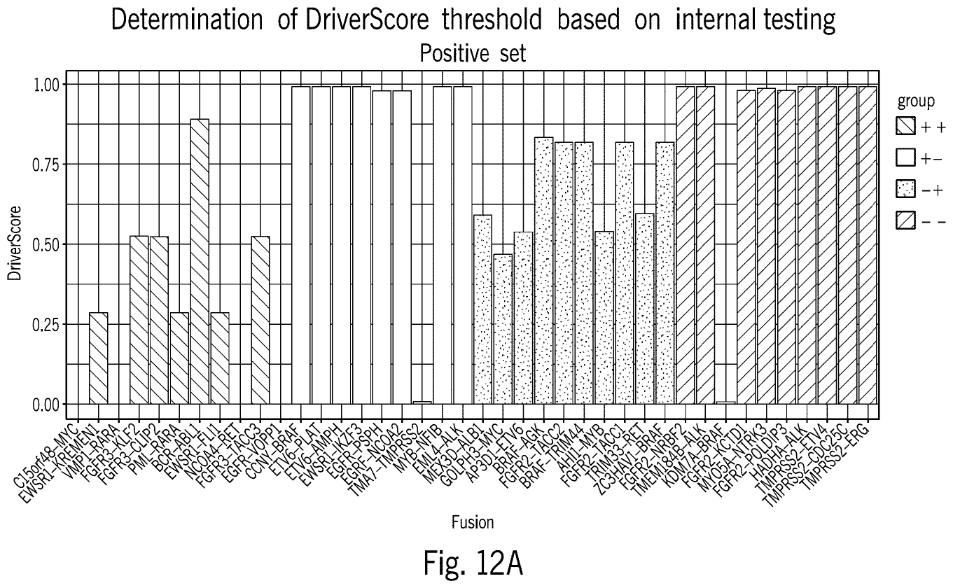

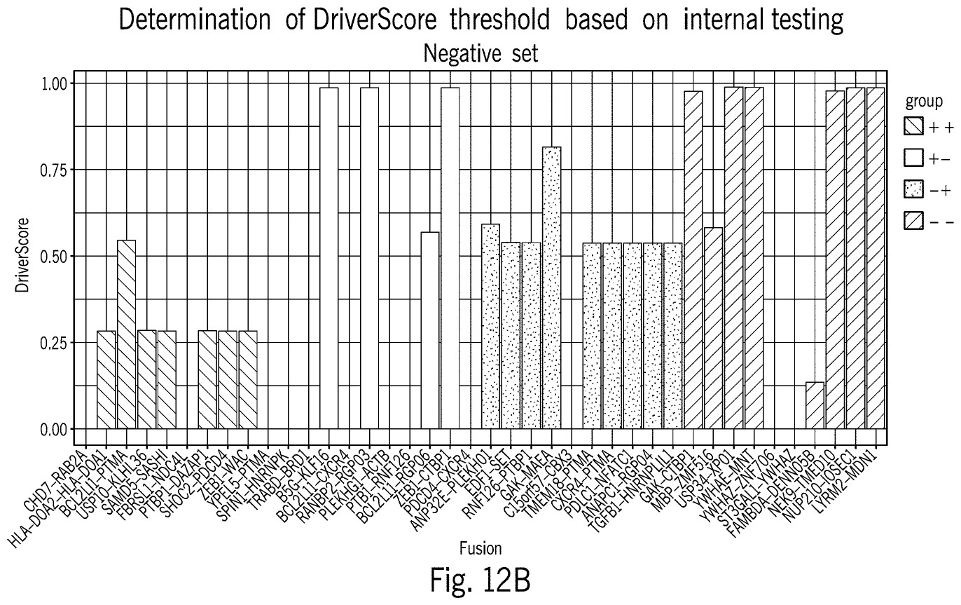

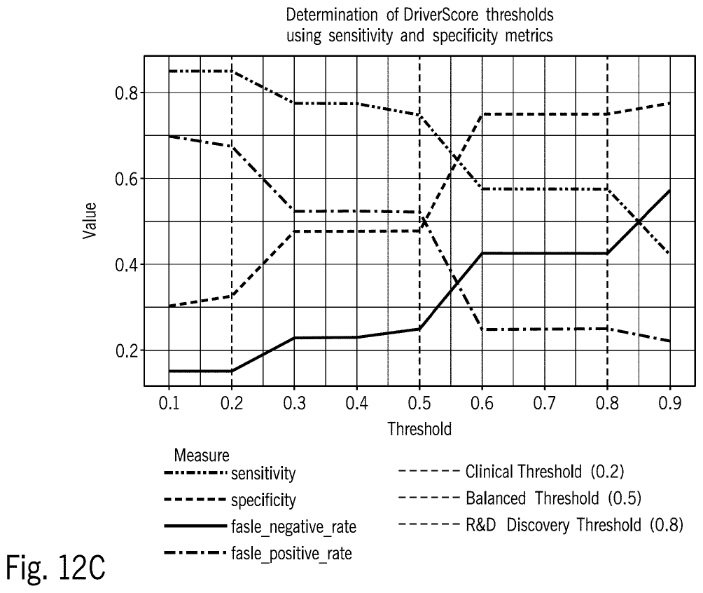

FIGS. 12A and 12B illustrate an example of positive and negative control testing sets, respectively, used to calculate performance metrics for the trained classifier 300. FIG. 12C illustrates an example of performance metric values plotted for each DriverScore threshold tested.

### Example: Optional Validation

RNA fusion candidates from a clinical test cohort (n=19) were retrospectively analyzed using the fusion classification pipeline in which all fusion transcripts were categorized into High, Medium or Low Confidence levels based on DriverScore and read support level (plot on left). The balanced DriverScore threshold (0.5) was used while the threshold for RNA fusion read support (‘hq spanning breaks’) was selected by an analysis of curated clinical sample data. High confidence fusions exceeded both the DriverScore and read support threshold (occurring in the upper right quadrant of the scatterplot), while Medium confidence fusions exceeded either the DriverScore or read support threshold (occurring at either the upper left or lower right quadrants) while the Low confidence fusions did not meet either the DriverScore threshold or the read support threshold (occurring at the lower left quadrant of the plot). The RNA fusion transcripts which were reported (signed out on clinical reports by an expert, for example a pathologist) are colored pink points (all others are colored in turquoise). Sensitivity (75%) and specificity (60%) metrics were calculated from this data as shown in the confusion matrix on the right hand side in which prioritized (positive) predictions were fusion transcripts scoring in either the High or Medium confidence groups while deprioritized (negative) predictions were fusion transcripts scoring in the Low confidence group.

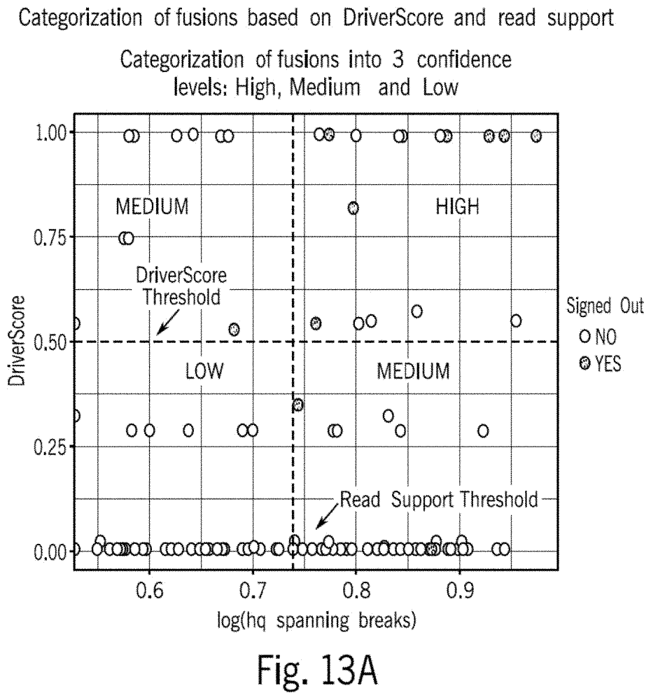

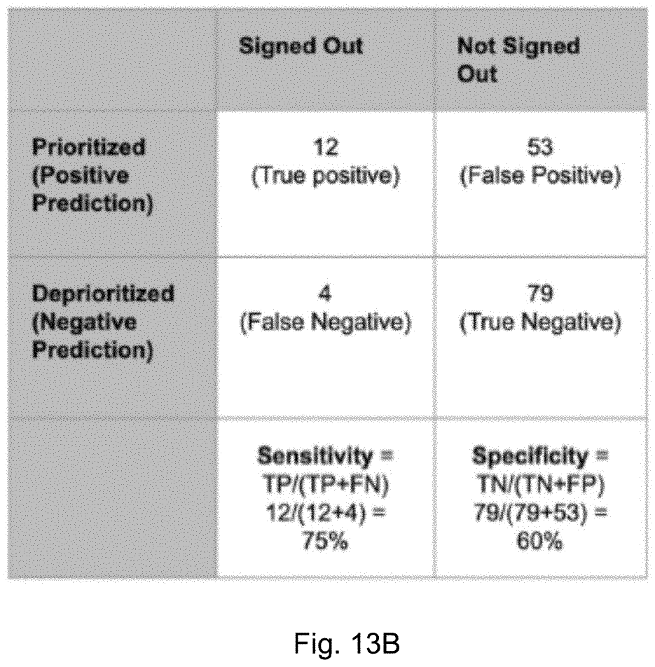

FIG. 13A illustrates an example of comparing trained classifier 300 output to thresholds to categorize fusions (as described at 530 in FIG. 5.). FIG. 13B illustrates a confusion matrix used for calculating performance metrics for classifier 300.

### Analysis Example

The fusion classification pipeline was used to retrospectively analyze all RNA fusion candidates detected in a specific clinical workflow during a span of two years. The number of fusion transcripts scoring in High, Medium or Low confidence group, along with a subset of unscored fusion events are enumerated in the left hand table with notes describing each group. The scatterplot on the right shows all the scored RNA fusion candidates plotted by their DriverScore and read support level (HQ Spanning Breaks) and colored by their Confidence category of High (pink), Medium (purple) or Low (blue). The balanced DriverScore threshold (0.5), shown in the dotted red line, achieved an overall sensitivity of 87% while the clinical DriverScore threshold, shown in the dotted blue line, achieved an overall sensitivity of 89%.

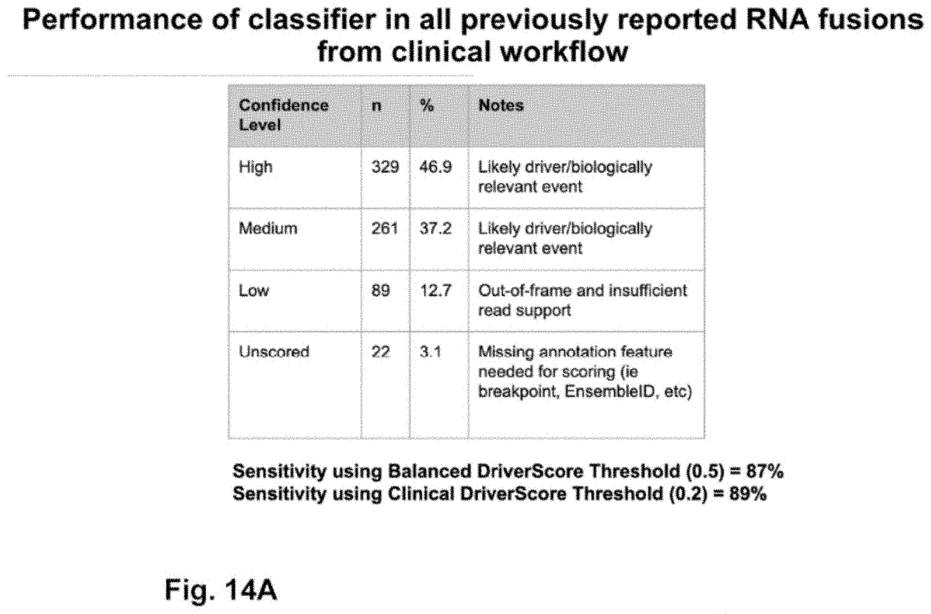

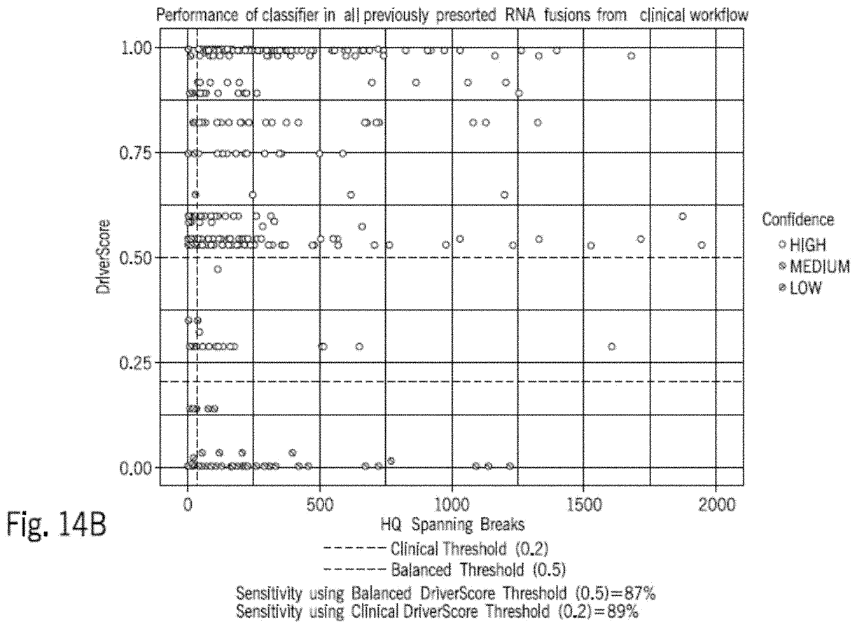

FIG. 14A illustrates results from analyzing a group of fusion candidates with trained classifier 300. FIG. 14B illustrates an example of comparing trained classifier 300 output to thresholds to categorize fusions (as described at 530 in FIG. 5.)

### Classification Example

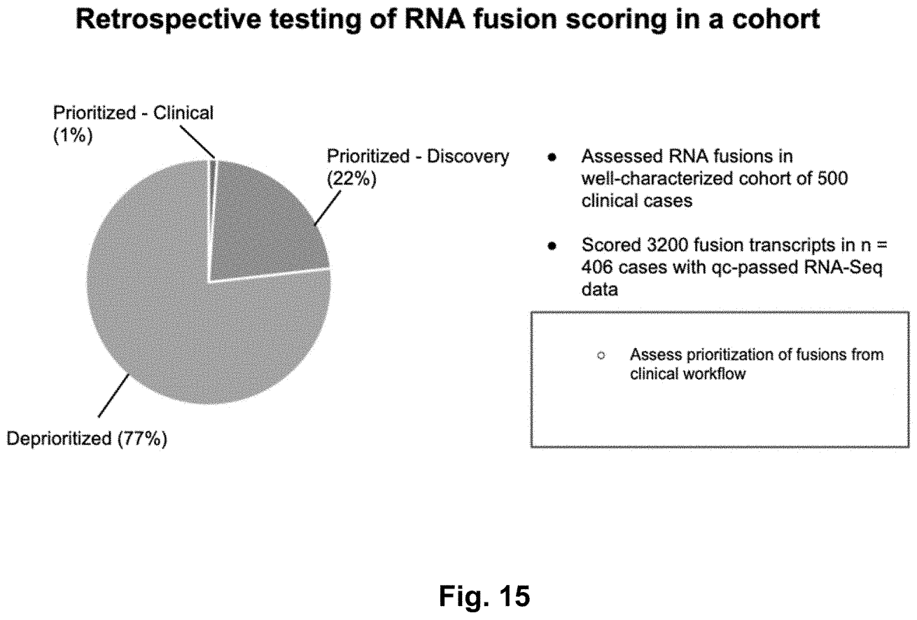

The fusion classification pipeline was used to retrospectively score all RNA fusion candidates in a previously published clinical cohort of 500 patients (Beaubier et al 2019, doi: 10.18632/oncotarget.26797). Specifically, 3200 RNA fusion candidates were analyzed from n=406 individuals in the cohort. This analysis was performed to a) assess the prioritization of RNA fusions in the clinical workflow and b) explore potential novel driver fusion events for R&D. The pie chart on the left of FIG. 15 shows that the majority of fusion events (77%) scored lowly and were deprioritized while those which had been clinically reported scored in the prioritized subset (1%) along with a proportion of highly scoring novel fusions (22%). FIG. 15 summarizes output from a trained classifier 300 for approximately 406 specimens associated with a combined total of approximately 3200 fusion events.

The methods and systems described above may be utilized in combination with or as part of a digital and laboratory health care platform that is generally targeted to medical care and research. It should be understood that many uses of the methods and systems described above, in combination with such a platform, are possible. One example of such a platform is described in U.S. Pre-Grant Publication No. 2021/0090694, published Mar. 25, 2021, titled “Data Based Cancer Research and Treatment Systems and Methods”, and filed Oct. 18, 2019, which is incorporated herein by reference and in its entirety for all purposes.

For example, an implementation of one or more embodiments of the methods and systems as described above may include microservices constituting a digital and laboratory health care platform supporting fusion pathogenicity scoring. Embodiments may include a single microservice for executing and delivering fusion pathogenicity ranking or may include a plurality of microservices each having a particular role which together implement one or more of the embodiments above. In one example, a first microservice may execute fusion data labeling in order to deliver labeled fusion data to a second microservice for fusion pathogenicity ranking. Similarly, the second microservice may execute fusion pathogenicity ranking to deliver a list of ranked fusion candidates according to an embodiment, above.

Where embodiments above are executed in one or more micro-services with or as part of a digital and laboratory health care platform, one or more of such micro-services may be part of an order management system that orchestrates the sequence of events as needed at the appropriate time and in the appropriate order necessary to instantiate embodiments above. A micro-services based order management system is disclosed, for example, in U.S. Prov. Patent Application No. 62/873,693, titled “Adaptive Order Fulfillment and Tracking Methods and Systems”, filed Jul. 12, 2019, U.S. patent application Ser. No. 16/789,288 filed Feb. 12, 2020 and published as U.S. Pre-Grant Publication No. 2021/0090694 on Aug. 13, 2020, and U.S. patent application Ser. No. 16/927,946 filed Jul. 13, 2020 and published as U.S. Pre-Grant Publication No. 2020/0365232 on Nov. 19, 2020, which are incorporated herein by reference and in their entirety for all purposes.

For example, continuing with the above first and second microservices, an order management system may notify the first microservice that an order for fusion data labeling has been received and is ready for processing. The first microservice may execute and notify the order management system once the delivery of labeled fusion data is ready for the second microservice. Furthermore, the order management system may identify that execution parameters (prerequisites) for the second microservice are satisfied, including that the first microservice has completed, and notify the second microservice that it may continue processing the order to rank fusion pathogenicity according to an embodiment, above.

Where the digital and laboratory health care platform further includes a genetic analyzer system, the genetic analyzer system may include targeted panels and/or sequencing probes. An example of a targeted panel is disclosed, for example, in U.S. Prov. Patent Application No. 62/902,950, titled “System and Method for Expanding Clinical Options for Cancer Patients using Integrated Genomic Profiling”, and filed Sep. 19, 2019, and U.S. patent application Ser. No. 15/930,234 filed May 12, 2020 and published as U.S. Pre-Grant Publication No. 2020/0365268 on Nov. 19, 2020, which are incorporated herein by reference and in their entirety for all purposes. In one example, targeted panels may enable the delivery of next generation sequencing results for fusion pathogenicity ranking according to an embodiment, above. An example of the design of next-generation sequencing probes is disclosed, for example, in U.S. Prov. Patent Application No. 62/924,073, titled “Systems and Methods for Next Generation Sequencing Uniform Probe Design”, and filed Oct. 21, 2019, which is incorporated herein by reference and in its entirety for all purposes.

Where the digital and laboratory health care platform further includes a bioinformatics pipeline, the methods and systems described above may be utilized after completion or substantial completion of the systems and methods utilized in the bioinformatics pipeline. As one example, the bioinformatics pipeline may receive next-generation genetic sequencing results and return a set of binary files, such as one or more BAM files, reflecting DNA and/or RNA read counts aligned to a reference genome. The methods and systems described above may be utilized, for example, to ingest the DNA and/or RNA read counts and produce ranked fusion data as a result.

When the digital and laboratory health care platform further includes an RNA data normalizer, any RNA read counts may be normalized before processing embodiments as described above. An example of an RNA data normalizer is disclosed, for example, in U.S. Pre-Grant Publication No. 2020/0098448 published on Mar. 26, 2020, which is incorporated herein by reference and in its entirety for all purposes.

When the digital and laboratory health care platform further includes a genetic data deconvoluter, any system and method for deconvoluting may be utilized for analyzing genetic data associated with a specimen having two or more biological components to determine the contribution of each component to the genetic data and/or determine what genetic data would be associated with any component of the specimen if it were purified. An example of a genetic data deconvoluter is disclosed, for example, in U.S. Pre-Grant Publication No. 2020/0210852 published on Jul. 2, 2020, U.S. Prov. Patent Application No. 62/924,054, titled “Calculating Cell-type RNA Profiles for Diagnosis and Treatment”, U.S. patent application Ser. No. 16/732,229 filed Dec. 31, 2019 and published as U.S. Pre-Grant Publication No. 2020/0210852 on Jul. 2, 2020, and U.S. patent application Ser. No. 17/074,984 filed Oct. 20, 2020 and published as U.S. Pre-Grant Publication No. 2021/0118526 on Apr. 22, 2021, and U.S. Prov. Patent Application No. 62/944,995, titled “Rapid Deconvolution of Bulk RNA Transcriptomes for Large Data Sets (Including Transcriptomes of Specimens Having Two or More Tissue Types)”, and filed Dec. 6, 2019, which are incorporated herein by reference and in their entirety for all purposes.

When the digital and laboratory health care platform further includes an automated RNA expression caller, RNA expression levels may be adjusted to be expressed as a value relative to a reference expression level, which is often done in order to prepare multiple RNA expression data sets for analysis to avoid artifacts caused when the data sets have differences because they have not been generated by using the same methods, equipment, and/or reagents. An example of an automated RNA expression caller is disclosed, for example, in U.S. Prov. Patent Application No. 62/943,712, titled “Systems and Methods for Automating RNA Expression Calls in a Cancer Prediction Pipeline”, and filed Dec. 4, 2019, and U.S. patent application Ser. No. 17/112,877 filed Dec. 4, 2020 which are incorporated herein by reference and in their entirety for all purposes.

The digital and laboratory health care platform may further include one or more insight engines to deliver information, characteristics, or determinations related to a disease state that may be based on genetic and/or clinical data associated with a patient and/or specimen. Exemplary insight engines may include a tumor of unknown origin engine, a human leukocyte antigen (HLA) loss of homozygosity (LOH) engine, a tumor mutational burden engine, a PD-L1 status engine, a homologous recombination deficiency engine, a cellular pathway activation report engine, an immune infiltration engine, a microsatellite instability engine, a pathogen infection status engine, a T cell receptor or B cell receptor profiling engine, and so forth. An example tumor of unknown origin engine is disclosed, for example, in U.S. Prov. Patent Application No. 62/855,750, titled “Systems and Methods for Multi-Label Cancer Classification”, and filed May 31, 2019, U.S. patent application Ser. No. 15/930,234 filed May 12, 2020 and published as U.S. Pre-Grant Publication No. 2020/0365268 on Nov. 19/2020, U.S. patent application Ser. No. 17/150,992 filed Jan. 15, 2021 and published as U.S. Pre-Grant Publication No. 2021/0142904 on May 13, 2021, which are incorporated herein by reference and in their entirety for all purposes. An example of an HLA LOH engine is disclosed, for example, in U.S. Pre-Grant Publication No. 2020/0258597 published on Aug. 13, 2020, which is incorporated herein by reference and in its entirety for all purposes. An example of a tumor mutational burden (TMB) engine is disclosed, for example, in U.S. Pre-Grant Publication No. 2020/0258601 published on Aug. 13, 2020, which is incorporated herein by reference and in its entirety for all purposes. An example of a PD-L1 status engine is disclosed, for example, in U.S. Prov. Patent Application No. 62/854,400, titled “A Pan-Cancer Model to Predict The PD-L1 Status of a Cancer Cell Sample Using RNA Expression Data and Other Patient Data”, and filed May, 30, 2019, and U.S. patent application Ser. No. 16/888,357 filed May. 29, 2020 and published as U.S. Pre-Grant Publication No. 2020/0395097 on Dec. 17, 2020, which are incorporated herein by reference and in their entirety for all purposes. An additional example of a PD-L1 status engine is disclosed, for example, in U.S. Prov. Patent Application No. 62/824,039, titled “PD-L1 Prediction Using H&E Slide Images”, and filed Mar. 26, 2019, and U.S. patent application Ser. No. 16/830,186 filed Mar. 25, 2020 and issued as U.S. Pat. No. 10,957,041 on Mar. 23, 2020, which are incorporated herein by reference and in their entirety for all purposes. An example of a homologous recombination deficiency engine is disclosed, for example, in U.S. Pat. No. 10,975,445 issued on Apr. 13, 2021, which is incorporated herein by reference and in its entirety for all purposes. An example of a cellular pathway activation report engine is disclosed, for example, in U.S. Prov. Patent Application No. 62/888,163, titled “Cellular Pathway Report”, and filed Aug. 16, 2019, and U.S. patent application Ser. No. 16/994,315 filed Aug. 14, 2020 and published as U.S. Pre-Grant Publication No. 2021/0057042 on Feb. 25, 2021, which are incorporated herein by reference and in their entirety for all purposes. An example of an immune infiltration engine is disclosed, for example, in U.S. Pre-Grant Publication No. 2020/0075169 published on Mar. 5, 2020, which is incorporated herein by reference and in its entirety for all purposes. An additional example of an immune infiltration engine is disclosed, for example, in U.S. Patent Application No. 62/804,509, titled “Comprehensive Evaluation of RNA Immune System for the Identification of Patients with an Immunologically Active Tumor Microenvironment”, and filed Feb. 12, 2019, which is incorporated herein by reference and in its entirety for all purposes. An example of an MSI engine is disclosed, for example, in U.S. Pre-Grant Publication No. 2020/0118644 on Apr. 16, 2020, which is incorporated herein by reference and in its entirety for all purposes. An additional example of an MSI engine is disclosed, for example, in U.S. Prov. Patent Application No. 62/931,600, titled “Systems and Methods for Detecting Microsatellite Instability of a Cancer Using a Liquid Biopsy”, and filed Nov. 6, 2019, and U.S. patent application Ser. No. 16/945,588 filed Jul. 31, 2020 and published as U.S. Pre-Grant Publication No. 2021/0098078 on Apr. 1, 2021, which are incorporated herein by reference and in their entirety for all purposes.

When the digital and laboratory health care platform further includes a report generation engine, the methods and systems described above may be utilized to create a summary report of a patient's genetic profile and the results of one or more insight engines for presentation to a physician. For instance, the report may provide to the physician information about the extent to which the specimen that was sequenced contained tumor or normal tissue from a first organ, a second organ, a third organ, and so forth. For example, the report may provide a genetic profile for each of the tissue types, tumors, or organs in the specimen. The genetic profile may represent genetic sequences present in the tissue type, tumor, or organ and may include variants, expression levels, information about gene products, or other information that could be derived from genetic analysis of a tissue, tumor, or organ. The report may include therapies and/or clinical trials matched based on a portion or all of the genetic profile or insight engine findings and summaries. For example, the clinical trials may be matched according to the systems and methods disclosed in U.S. Prov. Patent Application No. 62/855,913, titled “Systems and Methods of Clinical Trial Evaluation”, filed May 31, 2019, and U.S. patent application Ser. No. 16/889,779 filed Jun. 1, 2020 and published as U.S. Pre-Grant Publication No. 2020/0381087 on Dec. 3, 2020, which are incorporated herein by reference and in their entirety for all purposes.

The report may include a comparison of the results to a database of results from many specimens. An example of methods and systems for comparing results to a database of results are disclosed in U.S. Pre-Grant Publication No. 2020/0211716 published on Jul. 2, 2020, which is incorporated herein by reference and in its entirety for all purposes. The information may be used, sometimes in conjunction with similar information from additional specimens and/or clinical response information, to discover biomarkers or design a clinical trial.

When the digital and laboratory health care platform further includes application of one or more of the embodiments herein to organoids developed in connection with the platform, the methods and systems may be used to further evaluate genetic sequencing data derived from an organoid to provide information about the extent to which the organoid that was sequenced contained a first cell type, a second cell type, a third cell type, and so forth. For example, the report may provide a genetic profile for each of the cell types in the specimen. The genetic profile may represent genetic sequences present in a given cell type and may include variants, expression levels, information about gene products, or other information that could be derived from genetic analysis of a cell. The report may include therapies matched based on a portion or all of the deconvoluted information. These therapies may be tested on the organoid, derivatives of that organoid, and/or similar organoids to determine an organoid's sensitivity to those therapies. For example, organoids may be cultured and tested according to the systems and methods disclosed in U.S. patent application Ser. No. 16/693,117, titled “Tumor Organoid Culture Compositions, Systems, and Methods”, filed Nov. 22, 2019; U.S. Prov. Patent Application No. 62/924,621, titled “Systems and Methods for Predicting Therapeutic Sensitivity”, filed Oct. 22, 2019; U.S. Prov. Patent Application No. 62/944,292, titled “Large Scale Phenotypic Organoid Analysis”, filed Dec. 5, 2019; and U.S. Prov. Patent Application No. 63/012,885, titled “Systems and Methods for High Throughput Drug Screening”, filed Apr. 20, 2020 which are each incorporated herein by reference and in their entirety for all purposes.

When the digital and laboratory health care platform further includes application of one or more of the above in combination with or as part of a medical device or a laboratory developed test that is generally targeted to medical care and research, such laboratory developed test or medical device results may be enhanced and personalized through the use of artificial intelligence. An example of laboratory developed tests, especially those that may be enhanced by artificial intelligence, is disclosed, for example, in U.S. Provisional Patent Application No. 62/924,515, titled “Artificial Intelligence Assisted Precision Medicine Enhancements to Standardized Laboratory Diagnostic Testing”, and filed Oct. 22, 2019, and U.S. patent application Ser. No. 17/076,801 filed Oct. 21, 2020 and published as U.S. Pre-Grant Publication No. 2021/0118559 on Apr. 22, 2021, which are incorporated herein by reference and in their entirety for all purposes.

It should be understood that the examples given above are illustrative and do not limit the uses of the systems and methods described herein in combination with a digital and laboratory health care platform.

What is claimed is:

1. A method of categorizing fusions, comprising:
receiving labeled fusion data comprising at least one of DNA data or RNA data comprising at least one detected fusion associated with a specimen;
providing the labeled fusion data to a classifier trained to generate a pathogenicity metric corresponding to pathogenicity of each detected fusion;
receiving at least one pathogenicity metric from the classifier; and
generating a report comprising one or more detected fusions included in the at least one detected fusion based on the pathogenicity metrics.

2. The method of claim 1, wherein the pathogenicity metric is a numeric risk score in a range of 0 to 1.

3. The method of claim 2 further comprising:
generating a pathogenicity categorization corresponding to the labeled fusion data by comparing the pathogenicity metric to at least one predetermined threshold.

4. The method of claim 3, wherein the pathogenicity categorization is one of a low likelihood of pathogenicity, a medium likelihood of pathogenicity, or a high likelihood of pathogenicity.

5. The method of claim 1, wherein the pathogenicity metric is a pathogenicity categorization.

6. The method of claim 1, wherein the pathogenicity metric is one of a low likelihood of pathogenicity, a medium likelihood of pathogenicity, or a high likelihood of pathogenicity.

7. The method of claim 1, wherein the labeled fusion data comprises read data, and wherein the method further comprises:
generating a pathogenicity categorization corresponding to the labeled fusion data by:
comparing the pathogenicity metric to a first predetermined threshold; and
comparing the read data to a second predetermined threshold.

8. The method of claim 7, wherein the read data comprises at least one of a number of reads spanning a fusion breakpoint or a number of high quality reads spanning the fusion breakpoint.

9. The method of claim 1, wherein the labeled fusion data comprises at least one fusion having a 5′ partner sequence and a 3′ partner sequence, and for each fusion included in the at least one fusion, the labeled fusion data further comprises as least one of a Human Genome Organisation (HUGO) Gene Nomenclature Committee (HGNC) gene symbol for the 5′ partner sequence, a HGNC gene symbol for the 3′ partner sequence, an Ensembl ID for the 5′ partner sequence, an Ensembl ID for the 3′ partner sequence, a strandedness of the 5′ partner sequence, a strandedness of the 3′ partner sequence, a number or letter denoting a human chromosome on which the 5′ partner sequence is located, a number or letter denoting the human chromosome on which the 3′ partner sequence is located, a genomic coordinate start of the 5′ partner sequence, a genomic coordinate end of the 5′ partner sequence, a genomic coordinate start of the 3′ partner sequence, or a genomic coordinate end of the 3′ partner sequence.

10. The method of claim 9, wherein the strandedness of the 5′ partner sequence is one of forward or reverse.

11. The method of claim 9, wherein the strandedness of the 3′ partner sequence is one of forward or reverse.

12. The method of claim 1, wherein the classifier comprises a machine learning model.

13. The method of claim 1, wherein the classifier comprises at least one of a gradient boosting model, a random forest model, a neural network, a regression model, or a Naive Bayes model.

14. The method of claim 1, wherein the classifier is trained based on training data comprising a group of positive control fusions and a group of negative control fusions, each fusion included in the group of positive control fusions comprises a canonical fusion, and each fusion included in the group of negative control fusions is associated with healthy tissue.

15. The method of claim 1, wherein the classifier is previously trained by:
sequentially providing, for each of a number of fusions, a matrix of feature vectors to the classifier; and
sequentially updating, for each fusion included in the number of fusions, weights included in the classifier based on the matrix of feature vectors and a label associated with the fusion.

16. The method of claim 15 further comprising:
receiving, for each fusion included in the number of fusions, a pathogenicity score from the classifier;
determining model performance based on the pathogenicity score associated with each fusion, the label associated with each fusion, and a threshold; and
updating the threshold based on the model performance.

17. The method of claim 1 further comprising:
outputting the report to a physician.

18. The method of claim 1 further comprising:
flagging a fusion included in the labeled fusion data for review based on at least one of physician review or biological validation based on the labeled fusion data and the pathogenicity metric.

19. The method of claim 1, wherein the labeled fusion data is derived from next generation sequencing data.

20. The method of claim 1 further comprising:
categorizing a plurality of nucleic acid fusion events as oncogenic or not oncogenic based on at least one of a read level or an actionable fusion based on a knowledge database.

21. The method of claim 1, wherein the specimen is derived from a patient, and the report further comprises at least one therapy matched to the patient based on the detected fusions.

22. The method of claim 1, wherein the labeled fusion data comprises whole transcriptome RNA sequencing data generated by sequencing the specimen.

23. The method of claim 22, wherein the specimen comprises at least a portion of a tumor.

24. A fusion categorization system comprising at least one processor and at least one memory, the system configured to:
receive labeled fusion data comprising at least one of DNA data or RNA data comprising at least one detected fusion associated with a specimen;
provide the labeled fusion data to a classifier trained to generate a pathogenicity metric corresponding to pathogenicity of each detected fusion;
receive at least one pathogenicity metric from the classifier; and
generate a report comprising one or more detected fusions included in the at least one detected fusion based on the pathogenicity metrics.

25. A method of categorizing fusions, comprising:
receiving labeled fusion data comprising at least one of DNA data or RNA data comprising at least one detected fusion associated with a patient;
providing the labeled fusion data to a classifier trained to generate a pathogenicity metric corresponding to pathogenicity of each detected fusion;
receiving at least one pathogenicity metric from the classifier; and
generating a report comprising one or more detected fusions included in the at least one detected fusion based on the pathogenicity metrics.
